(19) United States
(12) Patent Application Publication
(10) Pub. No.: US 2005/0208494 A1
(43) Pub. Date: Sep. 22, 2005

(54) Novel proteins and dnas thereof

(75) Inventors: Atsushi Nakanishi, Tsukuba-shi (JP); Hiroyuki Miya, Tsukuba-shi (JP); Toshi Iwama, Tsukuba-shi (JP)

Correspondence Address: TAKEDA PHARMACEUTICALS NORTH AMERICA, INC;INTELLECTUAL PROPERTY DEPARTMENT, 475 HALF DAY ROAD, SUITE 500, LINCOLNSHIRE, IL, 60069, US

(73) Assignee: Takeda Pharmaceuticals North America, Inc., Lincolnshire, IL (US)

(21) Appl. No.: 10/514,258

(22) PCT Filed: May 13, 2003
(86) PCT No.: PCT/JP03/05941

§ 371 Date: Nov. 12, 2004

(30) Foreign Application Priority Data
May 14, 2002 (JP) 2002-139184
May 30, 2002 (JP) 2002-157669
Jul. 26, 2002 (JP) 2002-218324

(51) Int. Cl. C12Q 1/68; C07H 21/04; C12N 15/09; C07K 14/705; A61K 38/17

(52) U.S. Cl. 435/6; 435/69.1; 435/320.1; 435/325; 530/350; 514/12; 536/23.5

(57) ABSTRACT

A protein having an amino acid sequence which is the same or substantially the same as an amino acid sequence represented by SEQ ID NO:1, SEQ ID:8, SEQ ID NO:11 or SEQ ID NO:15 is useful as, for example, a diagnostic marker for digestive diseases, immune diseases associating thymic disorders, pancreatic diseases, diabetes, genital diseases, central nerve system diseases, circulatory diseases, muscular diseases, cancer, etc. A compound promoting or inhibiting the activity of this protein which is obtained by a screening method with the use of the protein is usable as, for example, a preventive or a remedy for the above diseases.

### TECHNICAL FIELD

The present invention provides a novel Ca²⁺ dependent K⁺ channel protein, a polynucleotide encoding the protein, a method for screening a compound that enhances or inhibits activities of the protein, a compound obtained by the screening method and the like.

### BACKGROUND ART

K⁺ channel, which is a membrane protein that selectively transmits a K⁺ ion, is generally present in prokaryotes to human and consists of numerous families. In vivo, it distributes in comprehensive tissues, and closely relates to important physiological functions such as generation of the resting membrane potential of cells, re-polarization, regulation of frequency for generating action potential and the like.

K⁺ channel has a huge variety of structures. In addition to six transmembrane type, two transmembrane type and one transmembrane type, a combined type channel molecule, wherein two basic structures are combined, is present.

Ca²⁺ dependent K⁺ channel (KCa) belongs to the six transmembrane type together with potential dependent K⁺ channel, and is classified into three families, Big-K (BK), Intermediate-K (IK) and Small-K (SK) based on channel conductance. The conductance of BK is 100-220 pS. BK is alternatively referred to as maxi-K or Slo (since Big-K from Drosophila is a gene accounting for the mutant Slowpoke). The human Slo was cloned in 1994 (Mol. Brain Res., Vol. 27, pp. 189-193, 1994). The conductance of IK and SK are 20-85 pS and 2-20 pS, respectively. The human IK1 (Proc. Natl. Acad. Sci. USA, Vol. 94, pp. 11651-11656, 1997) and the human SK1 (Science, Vol. 273, pp. 1709-1714, 1996) were cloned. In either family, pore region is present in the region between the fifth and the sixth transmembrane regions. In addition, it is considered that a C-terminal portion thereof, which is located at the inside of the cell, plays a role of Ca²⁺ sensitivity. The C-terminal portion is essential for expression of the function. By splicing the region, channels, which possess different Ca²⁺ dependencies, are formed. In K_{Ca}, main unit for channel is α subunit. However, only recently, it has been found that in the channel, β subunit is present as well as that of potential dependent K⁺ channel (Proc. Natl. Acad. Sci. USA, Vol. 93, pp. 9200-9205, 1996). The β subunit has two transmembrane regions. It is presumed that the β subunit has a structure, wherein the N-terminus and the C-terminus are present inside the cells. Further, it is considered that the β subunit plays a role of regulating the a subunit.

Since the human SK is highly expressed in dopamine-actuated mesocephalic nerve cells, it is considered that the Ca²⁺ dependent K⁺ channel plays a physiological function in the nerve cells. In addition, since dysfunction of the nerve cells is observed in schizophrenia, relation between schizophrenia and the human SK is pointed out (J. Neurosci., Vol. 21, pp. 3443-3456, 2001). It has been reported that the human IK1 relates to volume regulation of erythrocyte (Current. Opin. Hematol., Vol. 4, pp. 122-127, 1997) and regulation of secretion of Cl⁻ in small intestine (J. Clin. Invest., Vol. 98, pp. 2066-2075, 1996). Further, it has been known that IK1 inhibitor has a soothing effect on secretory diarrhea (J. Clin. Invest., Vol. 100, pp. 3111-3120, 1997).

For chicken-derived Ca²⁺ dependent K⁺ channel, cSlack has been reported (GenBank Accession No. AAM18770).

It is considered that K_{Ca} perceives membrane potential of cells and intracellular Ca²⁺ ion concentration, and regulates transmission of K⁺ ion. On the other hand, for K_{Ca}, various molecules, wherein channel conductance, sensitivity for Ca²⁺, response against channel activity regulating factor such as protein phosphorylase, and the like are different, are present. Their expression sites are also diverse. It is considered that K_{Ca} plays various important roles for the living body such as muscle contraction, incretion, transmission of K⁺ ion in kidney, signal transduction in nerve, etc. However, for the relation between each molecule and function thereof, there are various uncertain factors. Therefore, elucidation of the uncertainty may be conductive to development of medicament for treating various diseases relevant to K_{Ca}.

### DISCLOSURE OF THE INVENTION

The inventors have been intent on the study for solving the above-mentioned problems, and have found a novel Ca²⁺ dependent K⁺ channel protein. For a method of regulating the protein, it includes, for example, inhibition or enhancement of K⁺ ion transmission, lowering of expression level by suppression of transcription for the gene encoding the protein, activation of the promoter for the gene encoding the protein, accentuation of expression level by stabilizing mRNA, and the like. Based on the findings, the inventors have further studied and attained to perfection of the present invention.

That is, the present invention provides:
(1) A protein comprising the same or substantially the same amino acid sequence as the amino acid sequence represented by SEQ ID NO: 1, SEQ ID NO: 8, SEQ ID NO: 11 or SEQ ID NO: 15, or a salt thereof;
(2) The protein according to (1) or a salt thereof, wherein the protein comprising the same or substantially the same amino acid sequence as the amino acid sequence represented by SEQ ID NO: 1, SEQ ID NO: 8, SEQ ID NO: 11 or SEQ ID NO: 15 is a protein consisting of the amino acid sequence represented by SEQ ID NO: 18, SEQ ID NO: 23 or SEQ ID NO: 26;
(3) A protein consisting of the amino acid sequence represented by SEQ ID NO: 1, or a salt thereof;
(4) A protein consisting of the amino acid sequence represented by SEQ ID NO: 8, or a salt thereof;
(5) A protein consisting of the amino acid sequence represented by SEQ ID NO: 11, or a salt thereof;
(6) A protein consisting of the amino acid sequence represented by SEQ ID NO: 15, or a salt thereof;
(7) A partial peptide of the protein according to claim 1, or a salt thereof;
(8) A polynucleotide comprising the polynucleotide encoding the protein according to claim 1 or the partial peptide according to (7);
(9) The polynucleotide according to (8), which is a DNA;
(10) A polynucleotide consisting of the base sequence represented by SEQ ID NO: 2, SEQ ID NO: 9, SEQ ID NO: 12 or SEQ ID NO: 16;
(11) A polynucleotide consisting of the base sequence represented by SEQ ID NO: 19, SEQ ID NO: 24 or SEQ ID NO: 27;
(12) A recombinant vector comprising the polynucleotide according to (8);
(13) A transformant, which is transformed with the recombinant vector according to (12);
(14) A method for manufacturing the protein according to (1) or the partial peptide according to (7) or salts thereof, which comprises culturing the transformant according to (13), producing and accumulating the protein according to (1) or the partial peptide according to (7) and collecting them;
(15) A medicament comprising the protein according to (1) or the partial peptide according to (7) or salts thereof;
(16) A medicament comprising the polynucleotide according to (8);
(17) A diagnostic comprising the polynucleotide according to (8);
(18) An antibody to the protein according to (1) or the partial peptide according to (7) or salts thereof;
(19) A diagnostic comprising the antibody according to (18);
(20) A medicament comprising the antibody according to (18);
(21) A polynucleotide comprising a base sequence complementary or substantially complementary to the base sequence of the polynucleotide according to (8) or a portion thereof;
(22) A medicament comprising the polynucleotide according to (21);
(23) A method for screening a compound that enhances or inhibits activities of the protein according to (1) or the partial peptide according to (7) or salts thereof, or a salt thereof, which comprises using the protein according to (1) or the partial peptide according to (7) or salts thereof;
(24) A kit for screening a compound that enhances or inhibits activities of the protein according to (1) or the partial peptide according to (7) or salts thereof, or a salt thereof, which comprises the protein according to (1) or the partial peptide according to (7) or salts thereof;
(25) A compound that enhances or inhibits activities of the protein according to (1) or the partial peptide according to (7) or salts thereof, or a salt thereof, which is obtained by using the screening method according to (23) or the screening kit according to (24);
(26) A medicament comprising the compound according to (25) or a salt thereof;
(27) A method for screening a compound that enhances or inhibits an expression of the gene encoding the protein according to (1), which comprises using the polynucleotide according to (8);
(28) A kit for screening a compound that enhances or inhibits an expression of the gene encoding the protein according to (1), which comprises the polynucleotide according to (8);
(29) A compound that enhances or inhibits an expression of the gene encoding the protein according to (1), which is obtained by the screening method according to (27) or the screening kit according to (28);
(30) A medicament comprising the compound according to (29) or a salt thereof;
(31) A method for quantifying the protein according to (1), which comprises using the antibody according to (18);
(32) A method for diagnosing a disease associated with the function of the protein according to (1), which comprises using the quantification method according to (31);
(33) A method for screening a compound that enhances or inhibits an expression of the gene encoding the protein according to (1), which comprises using the antibody according to (18);
(34) A kit for screening a compound that enhances or inhibits an expression of the gene encoding the protein according to (1), which comprises the polynucleotide according to (18);
(35) A compound that enhances or inhibits an expression of the gene encoding the protein according to (1), which is obtained by the screening method according to (33) or the screening kit according to (34);
(36) A medicament comprising the compound according to (35) or a salt thereof;
(37) The medicament according to (16), (20), (22), (26), (30) or (36), which is a preventive and/or therapeutic agent for alimentary diseases, genital diseases or respiratory diseases;
(38) The diagnostic according to (17) or (19), which is a diagnostic for alimentary diseases, genital diseases or respiratory diseases;
(39) A preventing and/or treating method for alimentary diseases, genital diseases or respiratory diseases, which comprises administrating to mammal an effective amount of the compound according to (25), (29) or (35) or salts thereof;
(40) Use of the compound according to (25), (29) or (35) or salts thereof for manufacturing a preventive and/or therapeutic agent for alimentary diseases, genital diseases or respiratory diseases; and the like.

### BRIEF DESCRIPTION OF THE DRAWINGS

FIG. 1 is a drawing that shows a comparison of the amino acid sequences among human TCH204 variant 1 protein, KIAA1422 and Slack. In the figure, TCH204V1 shows the amino acid sequence represented by SEQ ID NO: 1. S1 to S6 and P indicate transmembrane regions and a pore region, respectively. The amino acid residues embraced with square show amino acid residues identical to TCH204V1. (continued to FIG. 2)

FIG. 2 is a drawing that shows a comparison of the amino acid sequences among human TCH204 variant 1 protein, KIAA1422 and Slack. In the figure, TCH204V1 shows the amino acid sequence represented by SEQ ID NO: 1. S1 to S6 and P indicate transmembrane regions and a pore region, respectively. The amino acid residues embraced with square show amino acid residues identical to TCH204V1. (continued from FIG. 1 and continued to FIG. 3)

FIG. 3 is a drawing that shows a comparison of the amino acid sequences among human TCH204 variant 1 protein, KIAA1422 and Slack. In the figure, TCH204V1 shows the amino acid sequence represented by SEQ ID NO: 1. S1 to S6 and P indicate transmembrane regions and a pore region, respectively. The amino acid residues embraced with square show amino acid residues identical to TCH204V1. (continued from FIG. 2)

FIG. 4 is a drawing that shows a comparison of the amino acid sequences among human TCH204 variant 1 protein, human TCH204 variant 2 protein, human TCH204 variant 3 protein and human TCH204 variant 4 protein. In the figure, TCH204V1, TCH204V2, TCH204V3 and TCH204V4 represent the amino acid sequence of SEQ ID NO: 1, SEQ ID NO: 8, SEQ ID NO: 11 and SEQ ID NO: 15, respectively. S1 to S6 and P indicate transmembrane regions and a pore region, respectively. The amino acid residues embraced with square show amino acid residues identical to TCH204V1. (continued to FIG. 5)

FIG. 5 is a drawing that shows a comparison of the amino acid sequences among human TCH204 variant 1 protein, human TCH204 variant 2 protein, human TCH204 variant 3 protein and human TCH204 variant 4 protein. In the figure, TCH204V1, TCH204V2, TCH204V3 and TCH204V4 represent the amino acid sequence of SEQ ID NO: 1, SEQ ID NO: 8, SEQ ID NO: 11 and SEQ ID NO: 15, respectively. S1 to S6 and P indicate transmembrane regions and a pore region, respectively. The amino acid residues embraced with square show amino acid residues identical to TCH204V1. (continued from FIG. 4 and continued to FIG. 6)

FIG. 6 is a drawing that shows a comparison of the amino acid sequences among human TCH204 variant 1 protein, human TCH204 variant 2 protein, human TCH204 variant 3 protein and human TCH204 variant 4 protein. In the figure, TCH204V1, TCH204V2, TCH204V3 and TCH204V4 represent the amino acid sequence of SEQ ID NO: 1, SEQ ID NO: 8, SEQ ID NO: 11 and SEQ ID NO: 15, respectively. S1 to S6 and P indicate transmembrane regions and a pore region, respectively. The amino acid residues embraced with square show amino acid residues identical to TCH204V1. (continued from FIG. 5)

FIG. 7 is a drawing that shows a comparison of the amino acid sequences among mouse TCH204 variant 6 protein, human TCH204 variant 1 protein and human Slot 4 protein. In the figure, mTCH204V6, hTCH204V1 and WO0240649.4 represent the amino acid sequence of SEQ ID NO: 18, SEQ ID NO: 1 and Slot 4, respectively. S1 to S6 and P indicate transmembrane regions and a pore region, respectively. The amino acid residues embraced with square show amino acid residues identical to mTCH204V6. (continued to FIG. 8)

FIG. 8 is a drawing that shows a comparison of the amino acid sequences among mouse TCH204 variant 6 protein, human TCH204 variant 1 protein and human Slot 4 protein. In the figure, mTCH204V6, hTCH204V1 and WO0240649.4 represent the amino acid sequence of SEQ ID NO: 18, SEQ ID NO: 1 and Slot 4, respectively. S1 to S6 and P indicate transmembrane regions and a pore region, respectively. The amino acid residues embraced with square show amino acid residues identical to mTCH204V6. (continued from FIG. 7 and continued to FIG. 9)

FIG. 9 is a drawing that shows a comparison of the amino acid sequences among mouse TCH204 variant 6 protein, human TCH204 variant 1 protein and human Slot 4 protein. In the figure, mTCH204V6, hTCH204V1 and WO0240649.4 represent the amino acid sequence of SEQ ID NO: 18, SEQ ID NO: 1 and Slot 4, respectively. S1 to S6 and P indicate transmembrane regions and a pore region, respectively. The amino acid residues embraced with square show amino acid residues identical to mTCH204V6. (continued from FIG. 8)

FIG. 10 is a drawing that shows a comparison of the amino acid sequences among mouse TCH204 variant 6 protein, mouse TCH204 variant 5 protein and mouse TCH204 variant 2 protein. In the figure, mTCH204V6, mTCH204V5 and mTCH204V2 represent the amino acid sequence of SEQ ID NO: 18, SEQ ID NO: 23 and SEQ ID NO: 26, respectively. S1 to S6 and P indicate transmembrane regions and a pore region, respectively. The amino acid residues embraced with square show amino acid residues identical to mTCH204V6. (continued to FIG. 11)

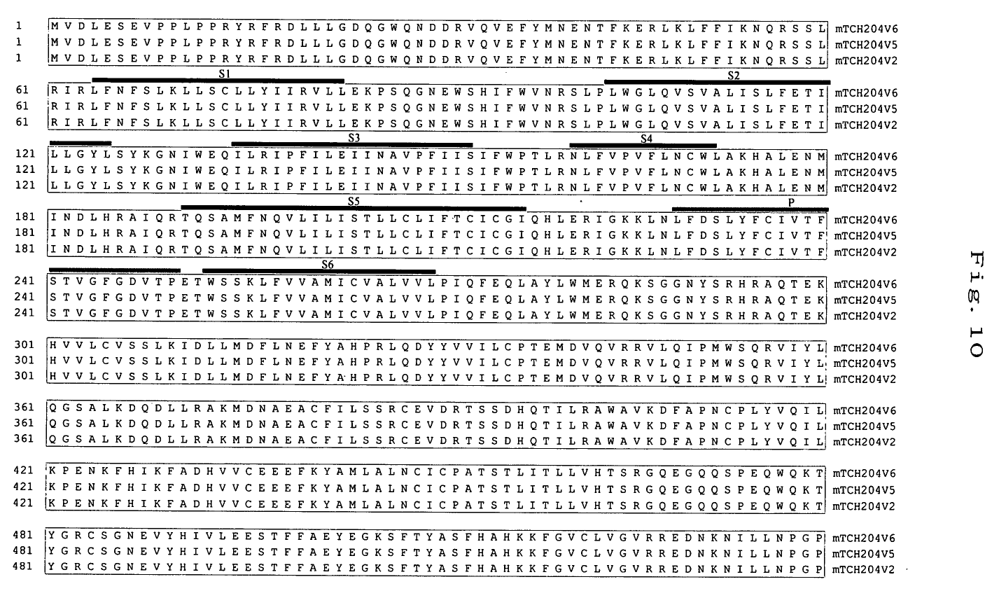

FIG. 11 is a drawing that shows a comparison of the amino acid sequences among mouse TCH204 variant 6 protein, mouse TCH204 variant 5 protein and mouse TCH204 variant 2 protein. In the figure, mTCH204V6, mTCH204V5 and mTCH204V2 represent the amino acid sequence of SEQ ID NO: 18, SEQ ID NO: 23 and SEQ ID NO: 26, respectively. S1 to S6 and P indicate transmembrane regions and a pore region, respectively. The amino acid residues embraced with square show amino acid residues identical to mTCH204V6. (continued from FIG. 11 and continued to FIG. 12)

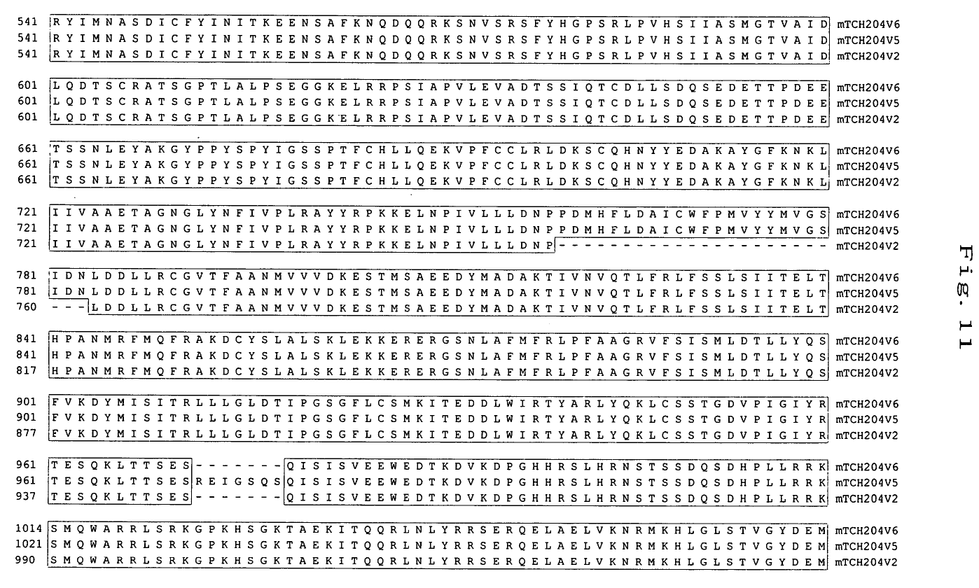

FIG. 12 is a drawing that shows a comparison of the amino acid sequences among mouse TCH204 variant 6 protein, mouse TCH204 variant 5 protein and mouse TCH204 variant 2 protein. In the figure, mTCH204V6, mTCH204V5 and mTCH204V2 represent the amino acid sequence of SEQ ID NO: 18, SEQ ID NO: 23 and SEQ ID NO: 26, respectively. 51 to S6 and P indicate transmembrane regions and a pore region, respectively. The amino acid residues embraced with square show amino acid residues identical to mTCH204V6. (continued from FIG. 11)

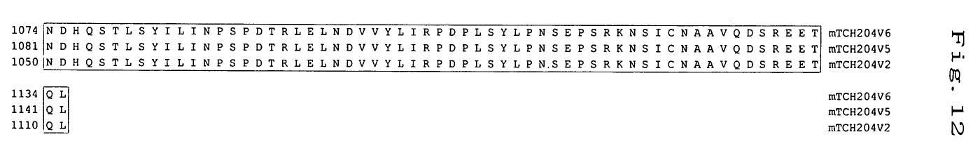

FIG. 13 is a drawing that shows an expression level of human TCH204 variant 1 gene in various human normal tissues derived cDNA. The relative expression level represented by ordinate indicates values multiplying the values obtained by dividing the expression level of the gene encoding the human TCH204 variant 1 per unit volume of cDNA (copies/μl) into the expression level of the GAPDH gene per unit volume of cDNA (copies/μl) by 100.

FIG. 14 is a drawing that shows an expression level of human TCH204 variant 2 gene in various human normal tissues derived cDNA. The relative expression level represented by ordinate indicates values multiplying the values obtained by dividing the expression level of the gene encoding the human TCH204 variant 2 per unit volume of cDNA (copies/μl) into the expression level of the GAPDH gene per unit volume of cDNA (copies/μl) by 100.

FIG. 15 is a drawing that shows an expression level of human TCH204 variant 3 gene in various human normal tissues derived cDNA. The relative expression level represented by ordinate indicates values multiplying the values obtained by dividing the expression level of the gene encoding the human TCH204 variant 3 per unit volume of cDNA (copies/μl) into the expression level of the GAPDH gene per unit volume of cDNA (copies/μl) by 100.

FIG. 16 is a drawing that shows an expression level of human TCH204 variant 4 gene in various human normal tissues derived cDNA. The relative expression level represented by ordinate indicates values multiplying the values obtained by dividing the expression level of the gene encoding the human TCH204 variant 4 per unit volume of cDNA (copies/μl) into the expression level of the GAPDH gene per unit volume of cDNA (copies/μl) by 100.

FIG. 17 is a drawing that shows an expression level of human TCH204 variant 5 gene in various human normal tissues derived cDNA. The relative expression level represented by ordinate indicates values multiplying the values obtained by dividing the expression level of the gene encoding the human TCH204 variant 5 per unit volume of cDNA (copies/μl) into the expression level of the GAPDH gene per unit volume of cDNA (copies/μl) by 100.

FIG. 18 is a drawing that shows an expression level of human TCH204 variant 6 gene in various human normal tissues derived cDNA. The relative expression level represented by ordinate indicates values multiplying the values obtained by dividing the expression level of the gene encoding the human TCH204 variant 6 per unit volume of cDNA (copies/μl) into the expression level of the GAPDH gene per unit volume of cDNA (copies/μl) by 100.

FIG. 19 is a drawing that shows a current-potential curve where the TCH204 variant 2 was transiently expressed in CHO-K1 cells. The membrane potential was held at −80 mV. Closed circles and open circles represent average values in the cells, in which the human TCH204 variant 2 vector was introduced (7 examples), and the cells, in which only the vector was introduced (5 examples), respectively.

### BEST MODE FOR CARRYING OUT THE INVENTION

The protein containing the same or substantially the same amino acid sequence as the amino acid sequence represented by SEQ ID NO: 1, SEQ ID NO: 8, SEQ ID NO: 11 or SEQ ID NO: 15 (hereinafter referred to as the protein of the present invention or the protein used in the present invention) may be any protein derived from any cells (e.g., liver cells, splenocytes, nerve cells, glial cells, pancreatic β cells, bone marrow cells, mesangial cells, Langerhans' cells, epidermic cells, epithelial cells, goblet cells, endothelial cells, smooth muscle cells, fibroblasts, fibrocytes, myocytes, fat cells, immune cells (e.g., macrophage, T cells, B cells, natural killer cells, mast cells, neutrophil, basophil, eosinophil, monocyte), megakaryocyte, synovial cells, chondrocytes, bone cells, osteoblasts, osteoclasts, mammary gland cells, hepatocytes or interstitial cells, the corresponding precursor cells, stem cells, cancer cells, etc.), or any tissues where such cells are present, e.g., brain or any region of the brain (e.g., olfactory bulb, amygdaloid nucleus, basal ganglia, hippocampus, thalamus, hypothalamus, cerebral cortex, medulla oblongata, cerebellum), spinal cord, hypophysis, stomach, pancreas, kidney, liver, gonad, thyroid, gall-bladder, bone marrow, adrenal gland, skin, muscle, lung, gastrointestinal tract (e.g., large intestine and small intestine), blood vessel, heart, thymus, spleen, submandibular gland, peripheral blood, peripheral blood, prostate, testis, ovary, placenta, uterus, bone, joint, skeletal muscle, etc. from human and other mammals (e.g., guinea pigs, rats, mice, chicken, rabbits, swine, sheep, bovine, monkeys, etc.). The protein may also be a synthetic protein.

The amino acid sequence which has substantially the same amino acid sequence as that represented by SEQ ID NO: 1 includes an amino acid sequence having at least about 75% homology, preferably at least about 80% homology, preferably at least about 85% homology, preferably at least about 90% homology, preferably at least about 95% homology, to the amino acid sequence represented by SEQ ID NO: 1.

Examples of the protein which contains substantially the same amino acid sequence as that represented by SEQ ID NO: 1 preferably include a protein containing substantially the same amino acid sequence as that represented by SEQ ID NO: 1 and having the activity substantially equivalent to the amino acid sequence represented by SEQ ID NO: 1, etc.

The amino acid sequence which has substantially the same amino acid sequence as that represented by SEQ ID NO: 8 includes an amino acid sequence having at least about 75% homology, preferably at least about 80% homology, preferably at least about 85% homology, preferably at least about 90% homology, preferably at least about 95% homology, preferably at least about 97% homology, to the amino acid sequence represented by SEQ ID NO: 8.

Examples of the protein which contains substantially the same amino acid sequence as that shown by SEQ ID NO: 8 preferably include a protein containing substantially the same amino acid sequence as that shown by SEQ ID NO: 8 and having the activity substantially equivalent to the amino acid sequence represented by SEQ ID NO: 8, etc. Specifically, it includes a protein containing the amino acid sequence represented by SEQ ID NO: 18, a protein containing the amino acid sequence represented by SEQ ID NO: 23, a protein containing the amino acid sequence represented by SEQ ID NO: 26, and the like.

The amino acid sequence which has substantially the same amino acid sequence as that represented by SEQ ID NO: 11 includes an amino acid sequence having at least about 75% homology, preferably at least about 80% homology, preferably at least about 85% homology, preferably at least about 90% homology, preferably at least about 95% homology, to the amino acid sequence represented by SEQ ID NO: 11.

Examples of the protein which contains substantially the same amino acid sequence as that represented by SEQ ID NO: 11 preferably include a protein containing substantially the same amino acid sequence as that represented by SEQ ID NO: 11 and having the activity substantially equivalent to the amino acid sequence represented by SEQ ID NO: 11, etc.

The amino acid sequence which has substantially the same amino acid sequence as that represented by SEQ ID NO: 15 includes an amino acid sequence having at least about 80% homology, preferably at least about 85% homology, preferably at least about 90% homology, preferably at least about 95% homology, to the amino acid sequence represented by SEQ ID NO: 15.

Examples of the protein which contains substantially the same amino acid sequence as that represented by SEQ ID NO: 15 preferably include a protein containing substantially the same amino acid sequence as that represented by SEQ ID NO: 15 and having the activity substantially equivalent to the amino acid sequence represented by SEQ ID NO: 15, etc.

Examples of the substantially equivalent activity include transmission of K⁺ ion, etc. The term “substantially equivalent” is used to mean that the nature of the activity is the same. Therefore, although it is preferred that the transmission of K⁺ ion be equivalent (e.g., about 0.01- to about 100-fold, preferably about 0.1- to about 10-fold, more preferably about 0.5- to about 2-fold), quantitative factors such as a level of the activity, a molecular weight of the protein, etc. may differ.

The transmission of K⁺ ion can be determined according to a publicly known method with some modifications, for example, by the methods described in Receptors and Channels, Vol. 6, pp. 337-350, 1999 or modified methods thereof.

Proteins containing the following amino acid sequences, which are so-called muteins, are used as the protein of the present invention: (1) (i) amino acid sequences represented by SEQ ID NO: 1, wherein at least 1 or 2 amino acids (approximately 1 to 300 amino acids, preferably approximately 1 to 200 amino acids, preferably approximately 1 to 150 amino acids, preferably approximately 1 to 100 amino acids, preferably approximately 1 to 50 amino acids, preferably approximately 1 to 30 amino acids, preferably approximately 1 to 10 amino acids, most preferably several (1 to 5) amino acids) are deleted; (ii) amino acid sequences represented by SEQ ID NO: 1, to which at least 1 or 2 amino acids (approximately 1 to 300 amino acids, preferably approximately 1 to 200 amino acids, preferably approximately 1 to 150 amino acids, preferably approximately 1 to 100 amino acids, preferably approximately 1 to 50 amino acids, preferably approximately 1 to 30 amino acids, preferably approximately 1 to 10 amino acids, most preferably several (1 to 5) amino acids) are added; (iii) amino acid sequences represented by SEQ ID NO: 1, in which at least 1 or 2 amino acids (approximately 1 to 300 amino acids, preferably approximately 1 to 200 amino acids, preferably approximately 1 to 150 amino acids, preferably approximately 1 to 100 amino acids, preferably approximately 1 to 50 amino acids, preferably approximately 1 to 30 amino acids, preferably approximately 1 to 10 amino acids, most preferably several (1 to 5) amino acids) are inserted; (iv) amino acid sequences represented by SEQ ID NO: 1, wherein at least 1 or 2 amino acids (approximately 1 to 300 amino acids, preferably approximately 1 to 200 amino acids, preferably approximately 1 to 150 amino acids, preferably approximately 1 to 100 amino acids, preferably approximately 1 to 50 amino acids, preferably approximately 1 to 30 amino acids, preferably approximately 1 to 10 amino acids, most preferably several (1 to 5) amino acids) are substituted by other amino acids; or (v) combination of the amino acid sequences described in the above, (2) (i) amino acid sequences represented by SEQ ID NO: 8, wherein at least 1 or 2 amino acids (approximately 1 to 300 amino acids, preferably approximately 1 to 200 amino acids, preferably approximately 1 to 150 amino acids, preferably approximately 1 to 100 amino acids, preferably approximately 1 to 50 amino acids, preferably approximately 1 to 30 amino acids, preferably approximately 1 to 10 amino acids, most preferably several (1 to 5) amino acids) are deleted; (ii) amino acid sequences represented by SEQ ID NO: 8, to which at least 1 or 2 amino acids (approximately 1 to 300 amino acids, preferably approximately 1 to 200 amino acids, preferably approximately 1 to 150 amino acids, preferably approximately 1 to 100 amino acids, preferably approximately 1 to 50 amino acids, preferably approximately 1 to 30 amino acids, preferably approximately 1 to 10 amino acids, most preferably several (1 to 5) amino acids) are added; (iii) amino acid sequences represented by SEQ ID NO: 8, in which at least 1 or 2 amino acids (approximately 1 to 300 amino acids, preferably approximately 1 to 200 amino acids, preferably approximately 1 to 150 amino acids, preferably approximately 1 to 100 amino acids, preferably approximately 1 to 50 amino acids, preferably approximately 1 to 30 amino acids, preferably approximately 1 to 10 amino acids, most preferably several (1 to 5) amino acids) are inserted; (iv) amino acid sequences represented by SEQ ID NO: 8, wherein at least 1 or 2 amino acids (approximately 1 to 300 amino acids, preferably approximately 1 to 200 amino acids, preferably approximately 1 to 150 amino acids, preferably approximately 1 to 100 amino acids, preferably approximately 1 to 50 amino acids, preferably approximately 1 to 30 amino acids, preferably approximately 1 to 10 amino acids, most preferably several (1 to 5) amino acids) are substituted by other amino acids; or (v) combination of the amino acid sequences described in the above, (3) (i) amino acid sequences represented by SEQ ID NO: 11, wherein at least 1 or 2 amino acids (approximately 1 to 300 amino acids, preferably approximately 1 to 200 amino acids, preferably approximately 1 to 150 amino acids, preferably approximately 1 to 100 amino acids, preferably approximately 1 to 50 amino acids, preferably approximately 1 to 30 amino acids, preferably approximately 1 to 10 amino acids, most preferably several (1 to 5) amino acids) are deleted; (ii) amino acid sequences represented by SEQ ID NO: 11, to which at least 1 or 2 amino acids (approximately 1 to 300 amino acids, preferably approximately 1 to 200 amino acids, preferably approximately 1 to 150 amino acids, preferably approximately 1 to 100 amino acids, preferably approximately 1 to 50 amino acids, preferably approximately 1 to 30 amino acids, preferably approximately 1 to 10 amino acids, most preferably several (1 to 5) amino acids) are added; (iii) amino acid sequences represented by SEQ ID NO: 11, in which at least 1 or 2 amino acids (approximately 1 to 300 amino acids, preferably approximately 1 to 200 amino acids, preferably approximately 1 to 150 amino acids, preferably approximately 1 to 100 amino acids, preferably approximately 1 to 50 amino acids, preferably approximately 1 to 30 amino acids, preferably approximately 1 to 10 amino acids, most preferably several (1 to 5) amino acids) are inserted; (iv) amino acid sequences represented by SEQ ID NO: 11, wherein at least 1 or 2 amino acids (approximately 1 to 300 amino acids, preferably approximately 1 to 200 amino acids, preferably approximately 1 to 150 amino acids, preferably approximately 1 to 100 amino acids, preferably approximately 1 to 50 amino acids, preferably approximately 1 to 30 amino acids, preferably approximately 1 to 10 amino acids, most preferably several (1 to 5) amino acids) are substituted by other amino acids; or (v) combination of the amino acid sequences described in the above, and (4) (i) amino acid sequences represented by SEQ ID NO: 15, wherein at least 1 or 2 amino acids (approximately 1 to 300 amino acids, preferably approximately 1 to 200 amino acids, preferably approximately 1 to 150 amino acids, preferably approximately 1 to 100 amino acids, preferably approximately 1 to 50 amino acids, preferably approximately 1 to 30 amino acids, preferably approximately 1 to 10 amino acids, most preferably several (1 to 5) amino acids) are deleted; (ii) amino acid sequences represented by SEQ ID NO: 15, to which at least 1 or 2 amino acids (approximately 1 to 300 amino acids, preferably approximately 1 to 200 amino acids, preferably approximately 1 to 150 amino acids, preferably approximately 1 to 100 amino acids, preferably approximately 1 to 50 amino acids, preferably approximately 1 to 30 amino acids, preferably approximately 1 to 10 amino acids, most preferably several (1 to 5) amino acids) are added; (iii) amino acid sequences represented by SEQ ID NO: 15, in which at least 1 or 2 amino acids (approximately 1 to 300 amino acids, preferably approximately 1 to 200 amino acids, preferably approximately 1 to 150 amino acids, preferably approximately 1 to 100 amino acids, preferably approximately 1 to 50 amino acids, preferably approximately 1 to 30 amino acids, preferably approximately 1 to 10 amino acids, most preferably several (1 to 5) amino acids) are inserted; (iv) amino acid sequences represented by SEQ ID NO: 15, wherein at least 1 or 2 amino acids (approximately 1 to 300 amino acids, preferably approximately 1 to 200 amino acids, preferably approximately 1 to 150 amino acids, preferably approximately 1 to 100 amino acids, preferably approximately 1 to 50 amino acids, preferably approximately 1 to 30 amino acids, preferably approximately 1 to 10 amino acids, most preferably several (1 to 5) amino acids) are substituted by other amino acids; or (v) combination of the amino acid sequences described in the above.

As described above, where an amino acid sequence is inserted, deleted or substituted, location of insertion, deletion or substitution is not restricted.

Throughout the present specification, the proteins are represented in accordance with the conventional way of describing peptides, that is, the N-terminus (amino terminus) at the left hand and the C-terminus (carboxyl terminus) at the right hand. In the proteins of the present invention including the proteins containing the amino acid sequence represented by SEQ ID NO: 1, the C-terminus may be in any form of a carboxyl group (—COOH), a carboxylate (—COO⁻), an amide (—CONH₂) or an ester (—COOR).

Examples of the ester group shown by R include a C₁₋₆ alkyl group such as methyl, ethyl, n-propyl, isopropyl, n-butyl, etc.; a C₃₋₈ cycloalkyl group such as cyclopentyl, cyclohexyl, etc.; a C₆₋₁₂ aryl group such as phenyl, a-naphthyl, etc.; a C₇₋₁₄ aralkyl group such as a phenyl-C₁₋₂-alkyl group, e.g., benzyl, phenethyl, etc., or an a-naphthyl-C₁₋₂-alkyl group such as a-naphthylmethyl, etc.; pivaloyloxymethyl and the like.

Where the protein of the present invention has a carboxyl group (or a carboxylate) at a position other than the C-terminus, it may be amidated or esterified and such an amide or ester is also included within the protein of the present invention. The ester group may be the same group as that described with respect to the C-terminus described above.

Furthermore, examples of the protein of the present invention include variants of the above proteins, wherein the amino group at the N-terminal amino acid residue (e.g., methionine residue) is protected with a protecting group (for example, a C₁₋₆ acyl group such as a C₁₋₆ alkanoyl group, e.g., formyl group, acetyl group, etc.); those wherein the N-terminal region is cleaved in vivo and the glutamyl group thus formed is pyroglutaminated; those wherein a substituent (e.g., —OH, —SH, amino group, imidazole group, indole group, guanidino group, etc.) on the side chain of an amino acid in the molecule is protected with a suitable protecting group (e.g., a C₁₋₆ acyl group such as a C₁₋₆ alkanoyl group, e.g., formyl group, acetyl group, etc.), or conjugated proteins such as glycoproteins bound to sugar chains.

Specific examples of the protein of the present invention include a protein containing an amino acid sequence represented by SEQ ID NO: 1, a protein containing an amino acid sequence represented by SEQ ID NO: 8, a protein containing an amino acid sequence represented by SEQ ID NO: 11, a protein containing an amino acid sequence represented by SEQ ID NO: 15, a protein containing an amino acid sequence represented by SEQ ID NO: 18, a protein containing an amino acid sequence represented by SEQ ID NO: 23, a protein containing an amino acid sequence represented by SEQ ID NO: 26, etc.

As partial peptides of the protein of the present invention, any partial peptide can be used so long as it can be a partial peptide of the protein of the present invention and preferably possess the same properties as that of the protein of the present invention described above.

Specifically, among the constituent amino acid sequence of the protein used in the present invention, a peptide, which comprises an amino acid sequence having at least 20 residues, preferably at least 50 residues, further preferably at least 70 residues, more preferably at least 100 residues and most preferably at least 200 residues, may be used.

The partial peptide used in the present invention may contain an amino acid sequence, wherein at least 1 or 2 amino acids (preferably approximately 1 to 10 amino acids, more preferably several (1 to 5) amino acids) are deleted; to which at least 1 or 2 amino acids (preferably approximately 1 to 20 amino acids, more preferably approximately 1 to 10 amino acids, and most preferably several (1 to 5) amino acids) are added; in which at least 1 or 2 amino acids (preferably approximately 1 to 10 amino acids, more preferably several and most preferably approximately 1 to 5 amino acids) are inserted; wherein at least 1 or 2 amino acids (preferably approximately 1 to 10 amino acids, more preferably several (1 to 5) amino acids) are substituted by other amino acids.

The partial peptide of the present invention includes, for example, a peptide having the 1st through the 63rd, the 84th through the 103rd, or the 271st through the 1118th amino acid sequence in the amino acid sequence represented by SEQ ID NO: 1; a peptide having the 1st through the 63rd, the 84th through the 103rd, or the 271st through the 1111st amino acid sequence in the amino acid sequence represented by SEQ ID NO: 8; a peptide having the 1st through the 63rd, the 84th through the 103rd, or the 271st through the 1061st amino acid sequence in the amino acid sequence represented by SEQ ID NO: 11; a peptide having the 1st through the 63rd, the 84th through the 103rd, or the 271st through the 759th amino acid sequence in the amino acid sequence represented by SEQ ID NO: 15; a peptide having the 1st through the 63rd, the 84th through the 103rd, or the 271st through the 1135th amino acid sequence in the amino acid sequence represented by SEQ ID NO: 18; a peptide having the 1st through the 63rd, the 84th through the 103rd, or the 271st through the 1142nd amino acid sequence in the amino acid sequence represented by SEQ ID NO: 23; a peptide having the 1st through the 63rd, the 84th through the 103rd, or the 271st through the 1111st amino acid sequence in the amino acid sequence represented by SEQ ID NO: 26; and the like.

In the partial peptide of the present invention, the C-terminus may be any of a carboxyl group (—COOH), a carboxylate (—COO⁻), an amide (—CONH₂) or an ester (—COOR).

As well as the aforementioned protein used in the present invention, the partial peptide of the present invention further includes those having carboxyl group (or carboxylate) apart from the C-terminus, those in which the amino group of the amino acid residue of the N-terminal methionine residue is protected by a protecting group, those in which the N-terminal residue is cleaved in vivo and the produced glutamine residue is pyroglutaminated, those in which substituents on the side chains of amino acids in the molecule are protected by appropriate protecting groups, conjugated peptides such as so-called glycoproteins, to which sugar chains are bound, and the like.

The partial peptide used in the present invention can also be used as an antigen for preparing an antibody. For example, to prepare the antibody of the present invention, which will be described below, the partial peptide includes a peptide having the 22nd through the 35th, the 84th through the 103rd, the 282nd through the 302nd, or the 656th through the 673rd amino acid sequence in the amino acid sequence represented by SEQ ID NO: 1; a peptide having the 22nd through the 35th, the 84th through the 103rd, the 282nd through the 302nd, or the 649th through the 666th amino acid sequence in the amino acid sequence represented by SEQ ID NO: 8; a peptide having the 22nd through the 35th, the 84th through the 103rd, the 282nd through the 302nd, or the 599th through the 616th amino acid sequence in the amino acid sequence represented by SEQ ID NO: 11; a peptide having the 22nd through the 35th, the 84th through the 103rd, the 282nd through the 302nd, or the 649th through the 666th amino acid sequence in the amino acid sequence represented by SEQ ID NO: 15; a peptide having the 22nd through the 35th, the 84th through the 103rd, the 282nd through the 302nd, or the 649th through the 666th amino acid sequence in the amino acid sequence represented by SEQ ID NO: 18; a peptide having the 22nd through the 35th, the 84th through the 103rd, the 282nd through the 302nd, or the 649th through the 666th amino acid sequence in the amino acid sequence represented by SEQ ID NO: 23; a peptide having the 22nd through the 35th, the 84th through the 103rd, the 282nd through the 302nd, or the 649th through the 666th amino acid sequence in the amino acid sequence represented by SEQ ID NO: 26; and the like.

For salts of the protein or the partial peptide of the present invention, preferred are salts with physiologically acceptable acids (e.g., inorganic acids, organic acids) or basses (e.g., alkali metal salts), especially physiologically acceptable acid addition salts. Examples of the salts include salts with, for example, inorganic acids (e.g., hydrochloric acid, phosphoric acid, hydrobromic acid, sulfuric acid); salts with organic acids (e.g., acetic acid, formic acid, propionic acid, fumaric acid, maleic acid, succinic acid, tartaric acid, citric acid, malic acid, oxalic acid, benzoic acid, methanesulfonic acid, benzenesulfonic acid) and the like.

The protein of the present invention or salts thereof may be manufactured by a publicly known method used to purify a protein from human and other mammalian cells or tissues described above, or by culturing a transformant that contains the DNA encoding the protein, as will be later described. Furthermore, the protein or its salts may also be manufactured by the methods for synthesizing proteins or by modifications thereof, which will also be described hereinafter.

Where the protein or its salts are manufactured from human or other mammalian tissues or cells, human or other mammalian tissues or cells are homogenized, then extracted with an acid or the like, and the extract obtained is isolated and purified by a combination of chromatography techniques such as reverse phase chromatography, ion exchange chromatography, and the like.

To synthesize the protein of the present invention, its partial peptide, or salts or amides thereof according to the present invention, commercially available resins that are used for protein synthesis may be used. Examples of such resins include chloromethyl resin, hydroxymethyl resin, benzhydrylamine resin, aminomethyl resin, 4-benzyloxybenzyl alcohol resin, 4-methylbenzhydrylamine resin, PAM resin, 4-hydroxymethylmehtylphenyl acetamidomethyl resin, polyacrylamide resin, 4-(2′,4′-dimethoxyphenylhydroxymethyl)phenoxy resin, 4-(2′,4′-dimethoxyphenyl-Fmoc-aminoethyl)phenoxy resin, etc. Using these resins, amino acids in which α-amino groups and functional groups on the side chains are appropriately protected are condensed on the resin in the order of the sequence of the objective protein according to various condensation methods publicly known in the art. At the end of the reaction, the receptor protein is cut out from the resin and at the same time, the protecting groups are removed. Then, intramolecular disulfide bond-forming reaction is performed in a highly diluted solution to obtain the objective protein or peptide, or amides thereof.

For condensation of the protected amino acids described above, a variety of activation reagents for protein synthesis may be used, and carbodiimides are particularly preferable. Examples of such carbodiimides include DCC, N,N′-diisopropylcarbodiimide, N-ethyl-N′-(3-dimethylaminoprolyl)carbodiimide, etc. For activation by these reagents, the protected amino acids in combination with a racemization inhibitor (e.g., HOBt, HOOBt) are added directly to the resin, or the protected amino acids are previously activated in the form of symmetric acid anhydrides, HOBt esters or HOOBt esters, followed by adding the thus activated protected amino acids to the resin.

Solvents suitable for use to activate the protected amino acids or condense with the resin may be chosen from solvents known to be usable for protein condensation reactions. Examples of such solvents are acid amides such as N,N-dimethylformamide, N,N-dimethylacetamide, N-methylpyrrolidone, etc.; halogenated hydrocarbons such as methylene chloride, chloroform, etc.; alcohols such as trifluoroethanol, etc.; sulfoxides such as dimethylsulfoxide, etc.; ethers such as pyridine, dioxane, tetrahydrofuran, etc.; nitriles such as acetonitrile, propionitrile, etc.; esters such as methyl acetate, ethyl acetate, etc.; and appropriate mixtures of these solvents. The reaction temperature is appropriately chosen from the range known to be applicable to protein binding reactions and is usually selected in the range of approximately −20° C. to 50° C. The activated amino acid derivatives are used generally in an excess of 1.5 to 4 times. The condensation is examined by a test using the ninhydrin reaction; when the condensation is insufficient, the condensation can be completed by repeating the condensation reaction without removal of the protecting groups. When the condensation is yet insufficient even after repeating the reaction, unreacted amino acids are acetylated with acetic anhydride or acetylimidazole.

Examples of the protecting groups used to protect the amino groups of the starting compounds include Z, Boc, t-pentyloxycarbonyl, isobornyloxycarbonyl, 4-methoxybenzyloxycarbonyl, Cl-Z, Br-Z, adamantyloxycarbonyl, trifluoroacetyl, phthaloyl, formyl, 2-nitrophenylsulphenyl, diphenylphosphinothioyl, Fmoc, etc.

A carboxyl group can be protected by, e.g., alkyl esterification (in the form of linear, branched or cyclic alkyl esters of the alkyl moiety such as methyl, ethyl, propyl, butyl, t-butyl, cyclopentyl, cyclohexyl, cycloheptyl, cyclooctyl, 2-adamantyl, etc.), aralkyl esterification (e.g., esterification in the form of benzyl ester, 4-nitrobenzyl ester, 4-methoxybenzyl ester, 4-chlorobenzyl ester, benzhydryl ester, etc.), phenacyl esterification, benzyloxycarbonyl hydrazidation, t-butoxycarbonyl hydrazidation, trityl hydrazidation, or the like.

A hydroxyl group of serine can be protected through, for example, its esterification or etherification. Examples of groups appropriately used for the esterification include a lower alkanoyl group, such as acetyl group, an aroyl group such as benzoyl group, and a group derived from carbonic acid such as benzyloxycarbonyl group, ethoxycarbonyl group, etc. Examples of a group appropriately used for the etherification include benzyl group, tetrahydropyranyl group, t-butyl group, etc.

Examples of groups for protecting the phenolic hydroxyl group of tyrosine include Bzl, Cl₂-Bzl, 2-nitrobenzyl, Br-Z, t-butyl, etc.

Examples of groups used to protect the imidazole moiety of histidine include Tos, 4-methoxy-2,3,6-trimethylbenzenesulfonyl, DNP, benzyloxymethyl, Bum, Boc, Trt, Fmoc, etc.

Examples of the activated carboxyl groups in the starting compounds include the corresponding acid anhydrides, azides, activated esters (esters with alcohols (e.g., pentachlorophenol, 2,4,5-trichlorophenol, 2,4-dinitrophenol, cyanomethyl alcohol, p-nitrophenol, HONB, N-hydroxysuccimide, N-hydroxyphthalimide, HOBt)). As the activated amino acids, in which the amino groups are activated in the starting material, the corresponding phosphoric amides are employed.

To eliminate (split off) the protecting groups, there are used catalytic reduction under hydrogen gas flow in the presence of a catalyst such as Pd-black or Pd-carbon; an acid treatment with anhydrous hydrogen fluoride, methanesulfonic acid, trifluoromethane-sulfonic acid or trifluoroacetic acid, or a mixture solution of these acids; a treatment with a base such as diisopropylethylamine, triethylamine, piperidine or piperazine; and reduction with sodium in liquid ammonia. The elimination of the protecting group by the acid treatment described above is carried out generally at a temperature of approximately −20° C. to 40° C. In the acid treatment, it is efficient to add a cation scavenger such as anisole, phenol, thioanisole, m-cresol, p-cresol, dimethylsulfide, 1,4-butanedithiol or 1,2-ethanedithiol. Furthermore, 2,4-dinitrophenyl group known as the protecting group for the imidazole of histidine is removed by a treatment with thiophenol. Formyl group used as the protecting group of the indole of tryptophan is eliminated by the aforesaid acid treatment in the presence of 1,2-ethanedithiol or 1,4-butanedithiol, as well as by a treatment with an alkali such as a dilute sodium hydroxide solution and dilute ammonia.

Protection of functional groups that should not be involved in the reaction of the starting materials, protecting groups, elimination of the protecting groups and activation of functional groups involved in the reaction may be appropriately selected from publicly known groups and publicly known means.

In another method for obtaining the amides of the protein, for example, the α-carboxyl group of the carboxy terminal amino acid is first protected by amidation; the peptide (protein) chain is then extended from the amino group side to a desired length. Thereafter, a protein in which only the protecting group of the N-terminal α-amino group in the peptide chain has been eliminated from the protein and a protein in which only the protecting group of the C-terminal carboxyl group has been eliminated are prepared. The two proteins are condensed in a mixture of the solvents described above. The details of the condensation reaction are the same as described above. After the protected protein obtained by the condensation is purified, all the protecting groups are eliminated by the method described above to give the desired crude protein. This crude protein is purified by various known purification means. Lyophilization of the major fraction gives the amide of the desired protein.

To prepare the esterified protein, for example, the α-carboxyl group of the carboxy terminal amino acid is condensed with a desired alcohol to prepare the amino acid ester, which is followed by procedure similar to the preparation of the amidated protein above to give the ester form of the desired protein.

The partial peptide or its salts in the protein of the present invention can be manufactured by publicly known methods for peptide synthesis, or by cleaving the protein of the present invention with an appropriate peptidase. For the methods for peptide synthesis, for example, either solid phase synthesis or liquid phase synthesis may be used. That is, the partial peptide or amino acids that can construct the protein of the present invention are condensed with the remaining part. Where the product contains protecting groups, these protecting groups are removed to give the desired peptide. Publicly known methods for condensation and elimination of the protecting groups are described in (a)-(e) below.
(a) M. Bodanszky & M. A. Ondetti: Peptide Synthesis, Interscience Publishers, New York (1966)
(b) Schroeder & Luebke: The Peptide, Academic Press, New York (1965)
(c) Nobuo Izumiya, et al.: Peptide Gosei-no-Kiso to Jikken (Basics and experiments of peptide synthesis), published by Maruzen Co. (1975)
(d) Haruaki Yajima & Shunpei Sakakibara: Seikagaku Jikkeii Koza (Biochemical Experiment) 1, Tanpakushitsu no Kagaku (Chemistry of Proteins) IV, 205 (1977)
(e) Haruaki Yajima, ed.: Zoku Iyakuhin no Kaihatsu (A sequel to Development of Pharmaceuticals), Vol. 14, Peptide Synthesis, published by Hirokawa Shoten

After completion of the reaction, the product may be purified and isolated by a combination of conventional purification methods such as solvent extraction, distillation, column chromatography, liquid chromatography and re-crystallization to give the partial peptide of the present invention. When the partial peptide obtained by the above methods is in a free form, the peptide can be converted into an appropriate salt by a publicly known method; when the protein is obtained in a salt form, it can be converted into a free form by a publicly known method.

The polynucleotide encoding the protein of the present invention may be any polynucleotide so long as it contains the base sequence encoding the protein of the present invention described above. Preferred is DNA. The DNA may be any of genomic DNA, genomic DNA library, cDNA derived from the cells and tissues described above, cDNA library derived from the cells and tissues described above and synthetic DNA.

The vector to be used for the library may be any of bacteriophage, plasmid, cosmid and phagemid. The DNA may also be directly amplified by reverse transcriptase polymerase chain reaction (hereinafter abbreviated as RT-PCR) using the total RNA or mRNA fraction prepared from the cells and tissues described above.

Specifically, the DNA encoding the protein of the present invention may be (1) (i) DNA having the base sequence shown by SEQ ID NO: 2, or DNA having the base sequence hybridizable to the base sequence represented by SEQ ID NO: 2 under highly stringent conditions and encoding a protein having the activities substantially equivalent to those of the protein containing the amino acid sequence represented by SEQ ID NO: 1, (ii) DNA having the base sequence shown by SEQ ID NO: 7, or DNA having the base sequence hybridizable to the base sequence represented by SEQ ID NO: 7 under highly stringent conditions and encoding a protein having the activities substantially equivalent to those of the protein containing the amino acid sequence represented by SEQ ID NO: 1; (2) (i) DNA having the base sequence shown by SEQ ID NO: 9, or DNA having the base sequence hybridizable to the base sequence represented by SEQ ID NO: 9 under highly stringent conditions and encoding a protein having the activities substantially equivalent to those of the protein containing the amino acid sequence represented by SEQ ID NO: 8, (ii) DNA having the base sequence shown by SEQ ID NO: 10, or DNA having the base sequence hybridizable to the base sequence represented by SEQ ID NO: 10 under highly stringent conditions and encoding a protein having the activities substantially equivalent to those of the protein containing the amino acid sequence represented by SEQ ID NO: 8; (3) (i) DNA having the base sequence shown by SEQ ID NO: 12, or DNA having the base sequence hybridizable to the base sequence represented by SEQ ID NO: 12 under highly stringent conditions and encoding a protein having the activities substantially equivalent to those of the protein containing the amino acid sequence represented by SEQ ID NO: 11, (ii) DNA having the base sequence shown by SEQ ID NO: 13, or DNA having the base sequence hybridizable to the base sequence represented by SEQ ID NO: 13 under highly stringent conditions and encoding a protein having the activities substantially equivalent to those of the protein containing the amino acid sequence represented by SEQ ID NO: 11; or (4) (i) DNA having the base sequence shown by SEQ ID NO: 16, or DNA having the base sequence hybridizable to the base sequence represented by SEQ ID NO: 16 under highly stringent conditions and encoding a protein having the activities substantially equivalent to those of the protein containing the amino acid sequence represented by SEQ ID NO: 15, (ii) DNA having the base sequence shown by SEQ ID NO: 17, or DNA having the base sequence hybridizable to the base sequence represented by SEQ ID NO: 17 under highly stringent conditions and encoding a protein having the activities substantially equivalent to those of the protein containing the amino acid sequence represented by SEQ ID NO: 15.

Examples of the DNA hybridizable to the base sequence represented by SEQ ID NO: 2 or SEQ ID NO: 7 under highly stringent conditions include DNA containing a base sequence having at least about 55% homology, preferably at least about 60% homology, preferably at least about 65% homology, preferably at least about 70% homology, preferably at least about 75% homology, preferably at least about 80% homology, preferably at least about 85% homology, preferably at least about 90% homology, and most preferably at least about 95% homology, to the base sequence represented by SEQ ID NO: 2 or SEQ ID NO: 7.

Examples of the DNA hybridizable to the base sequence represented by SEQ ID NO: 9 or SEQ ID NO: 10 under highly stringent conditions include DNA containing a base sequence having at least about 65% homology, preferably at least about 70% homology, preferably at least about 75% homology, preferably at least about 80% homology, preferably at least about 85% homology, preferably at least about 90% homology, and most preferably at least about 95% homology, to the base sequence represented by SEQ ID NO: 9 or SEQ ID NO: 10. Specifically, it includes a DNA containing the base sequence represented by SEQ ID NO: 19, a DNA containing the base sequence represented by SEQ ID NO: 24, a DNA containing the base sequence represented by SEQ ID NO: 27, or the like.

Examples of the DNA hybridizable to the base sequence represented by SEQ ID NO: 12 or SEQ ID NO: 13 under highly stringent conditions include DNA containing a base sequence having at least about 60% homology, preferably at least about 65% homology, preferably at least about 70% homology, preferably at least about 75% homology, preferably at least about 80% homology, preferably at least about 85% homology, preferably at least about 90% homology, and most preferably at least about 95% homology, to the base sequence represented by SEQ ID NO: 12 or SEQ ID NO: 13.

Examples of the DNA hybridizable to the base sequence represented by SEQ ID NO: 16 or SEQ ID NO: 17 under highly stringent conditions include DNA containing a base sequence having at least about 60% homology, preferably at least about 65% homology, preferably at least about 70% homology, preferably at least about 75% homology, preferably at least about 80% homology, preferably at least about 85% homology, preferably at least about 90% homology, and most preferably at least about 95% homology, to the base sequence represented by SEQ ID NO: 16 or SEQ ID NO: 17.

The hybridization can be carried out by publicly known methods or by modifications of these methods, for example, according to the method described in Molecular Cloning, 2nd (J. Sambrook et al., Cold Spring Harbor Lab. Press, 1989). A commercially available library may also be used according to the instructions of the attached manufacturer's protocol. Preferably, the hybridization can be carried out under highly stringent conditions.

The highly stringent conditions used herein are, for example, those in a sodium concentration at about 19 mM to about 40 mM, preferably about 19 mM to about 20 mM at a temperature of about 50° C. to about 70° C., preferably about 60° C. to about 65° C. In particular, hybridization conditions in a sodium concentration of about 19 mM at a temperature of about 65° C. are most preferred.

More specifically, for the DNA encoding the protein having the amino acid sequence represented by SEQ ID NO: 1, there may be employed DNA having the base sequence represented by SEQ ID NO: 2 or SEQ ID NO: 7. For the DNA encoding the protein having the amino acid sequence represented by SEQ ID NO: 8, there may be employed DNA having the base sequence represented by SEQ ID NO: 9 or SEQ ID NO: 10. For the DNA encoding the protein having the amino acid sequence represented by SEQ ID NO: 11, there may be employed DNA having the base sequence represented by SEQ ID NO: 12 or SEQ ID NO: 13. For the DNA encoding the protein having the amino acid sequence represented by SEQ ID NO: 15, there may be employed DNA having the base sequence represented by SEQ ID NO: 16 or SEQ ID NO: 17. For the DNA encoding the protein having the amino acid sequence represented by SEQ ID NO: 18, there may be employed DNA having the base sequence represented by SEQ ID NO: 19 or SEQ ID NO: 22. For the DNA encoding the protein having the amino acid sequence represented by SEQ ID NO: 23, there may be employed DNA having the base sequence represented by SEQ ID NO: 24 or SEQ ID NO: 25. For the DNA encoding the protein having the amino acid sequence represented by SEQ ID NO: 26, there may be employed DNA having the base sequence represented by SEQ ID NO: 27 or SEQ ID NO: 28.

For the DNA encoding a partial peptide used in the present invention, any DNA containing the base sequence encoding a partial peptide used in the present invention may be used. In addition, any one of genomic DNA, genomic DNA library, cDNA derived from the above described cells or tissues, cDNA library derived from the above described cells or tissues, synthetic DNA may be used.

For the DNA encoding the partial peptide used in the present invention, there may be employed DNA having a portion of the DNA having the base sequence represented by SEQ ID NO: 2 or SEQ ID NO: 7, or DNA containing the base sequence, which hybridizes to the base sequence represented by SEQ ID NO: 2 or SEQ ID NO: 7 under high stringent conditions, and containing a portion of the DNA encoding the protein having the activity substantially equivalent to that of the protein containing the amino acid sequence represented by SEQ ID NO: 1. For the DNA encoding the partial peptide used in the present invention, there may be employed DNA having a portion of the DNA having the base sequence represented by SEQ ID NO: 9 or SEQ ID NO: 10, or DNA containing the base sequence, which hybridizes to the base sequence represented by SEQ ID NO: 9 or SEQ ID NO: 10 under high stringent conditions, and containing a portion of the DNA encoding the protein having the activity substantially equivalent to that of the protein containing the amino acid sequence represented by SEQ ID NO: 8. For the DNA encoding the partial peptide used in the present invention, there may be employed DNA having a portion of the DNA having the base sequence represented by SEQ ID NO: 12 or SEQ ID NO: 13, or DNA containing the base sequence, which hybridizes to the base sequence represented by SEQ ID NO: 12 or SEQ ID NO: 13 under high stringent conditions, and containing a portion of the DNA encoding the protein having the activity substantially equivalent to that of the protein containing the amino acid sequence represented by SEQ ID NO: 11. For the DNA encoding the partial peptide used in the present invention, there may be employed DNA having a portion of the DNA having the base sequence represented by SEQ ID NO: 16 or SEQ ID NO: 17, or DNA containing the base sequence, which hybridizes to the base sequence represented by SEQ ID NO: 16 or SEQ ID NO: 17 under high stringent conditions, and containing a portion of the DNA encoding the protein having the activity substantially equivalent to that of the protein containing the amino acid sequence represented by SEQ ID NO: 15.

DNA hybridizable to the base sequence represented by SEQ ID NO: 2 or SEQ ID NO: 15, DNA hybridizable to the base sequence represented by SE ID NO: 9 or SEQ ID NO: 10, DNA hybridizable to the base sequence represented by SE ID NO: 12 or SEQ ID NO: 13, and DNA hybridizable to the base sequence represented by SE ID NO: 16 or SEQ ID NO: 17 show the same meaning as described above.

For a hybridization method and high stringent conditions, similar ones as described above are utilized.

For cloning of the DNA that completely encodes the protein of the present invention or its partial peptide (hereinafter sometimes collectively referred to as the protein of the present invention), the DNA may be either amplified by PCR using synthetic DNA primers containing a part of the base sequence of DNA encoding the protein of the present invention, or the DNA inserted into an appropriate vector can be selected by hybridization with a labeled DNA fragment or synthetic DNA that encodes a part or entire region of the protein of the present invention. The hybridization can be carried out, for example, according to the method described in Molecular Cloning, 2nd, J. Sambrook et al., Cold Spring Harbor Lab. Press, 1989. The hybridization may also be performed using commercially available library in accordance with the protocol described in the attached instructions.

Conversion of the base sequence of the DNA can be effected by publicly known methods such as the ODA-LA PCR method, the Gapped duplex method or the Kunkel method or its modification by using PCR or a publicly known kit available as Mutan™-super Express Km or Mutan™-K (both manufactured by Takara Shuzo Co., Ltd.).

The cloned DNA encoding the protein can be used as it is, depending upon purpose or, if desired, after digestion with a restriction enzyme or after addition of a linker thereto. The DNA may contain ATG as a translation initiation codon at the 5′ end thereof and may further contain TAA, TGA or TAG as a translation termination codon at the 3′ end thereof. These translation initiation and termination codons may also be added by using an appropriate synthetic DNA adapter.

The expression vector for the protein of the present invention can be manufactured, for example, by (a) excising the desired DNA fragment from the DNA containing the DNA encoding the protein of the present invention, and then (b) ligating the DNA fragment with an appropriate expression vector downstream a promoter in the vector.

Examples of the vector include plasmids derived form E. coli (e.g., pBR322, pBR325, pUC12, pUC13), plasmids derived from Bacillus subtilis (e.g., pUB110, pTP5, pC194), plasmids derived from yeast (e.g., pSH19, pSH15), bacteriophages such as ? phage, etc., animal viruses such as retrovirus, vaccinia virus, baculovirus, etc. as well as pA1-11, pXT1, pRc/CMV, pRc/RSV, pcDNAI/Neo, etc.

The promoter used in the present invention may be any promoter if it matches well with a host to be used for gene expression. In the case of using animal cells as the host, examples of the promoter include SRa promoter, SV40 promoter, LTR promoter, CMV promoter, HSV-TK promoter, etc.

Among them, CMV promoter or SRa promoter is preferably used. Where the host is bacteria of the genus Escherichia, preferred examples of the promoter include trp promoter, lac promoter, recA promoter, ?P_{L} promoter, 1pp promoter, T7 promoter, etc. In the case of using bacteria of the genus Bacillus as the host, preferred example of the promoter are SPO1 promoter, SPO2 promoter and penP promoter. When yeast is used as the host, preferred examples of the promoter are PHO5 promoter, PGK promoter, GAP promoter and ADH promoter. When insect cells are used as the host, preferred examples of the promoter include polyhedrin prompter and P10 promoter.

In addition to the foregoing examples, the expression vector may further optionally contain an enhancer, a splicing signal, a polyA addition signal, a selection marker, SV40 replication origin (hereinafter sometimes abbreviated as SV40ori) etc. Examples of the selection marker include dihydrofolate reductase (hereinafter sometimes abbreviated as dhfr) gene [methotrexate (MTX) resistance], ampicillin resistant gene (hereinafter sometimes abbreviated as Ampr), neomycin resistant gene (hereinafter sometimes abbreviated as Neo^{r}, G418 resistance), etc. In particular, when dhfr gene is used as the selection marker in CHO (dhfr⁻) cells, selection can also be made on thymidine free media.

If necessary and desired, a signal sequence that matches with a host is added to the N-terminus of the protein of the present invention. Examples of the signal sequence that can be used are PhoA signal sequence, OmpA signal sequence, etc. in the case of using bacteria of the genus Escherichia as the host; a-amylase signal sequence, subtilisin signal sequence, etc. in the case of using bacteria of the genus Bacillus as the host; MFa signal sequence, SUC2 signal sequence, etc. in the case of using yeast as the host; and insulin signal sequence, a-interferon signal sequence, antibody molecule signal sequence, etc. in the case of using animal cells as the host, respectively.

As described above, using the vector containing the DNA encoding the protein of the present invention thus constructed, transformants can be manufactured.

Examples of the host, which may be employed, are bacteria belonging to the genus Escherichia, bacteria belonging to the genus Bacillus, yeast, insect cells, insects and animal cells, etc.

Specific examples of the bacteria belonging to the genus Escherichia include Escherichia coli K12 DH1 (Proc. Natl. Acad. Sci. U.S.A., 60, 160 (1968)), JM103 (Nucleic Acids Research, 9, 309 (1981)), JA221 (Journal of Molecular Biology, 120, 517 (1978)), HB101 (Journal of Molecular Biology, 41, 459 (1969)), C600 (Genetics, 39, 440 (1954)), etc.

Examples of the bacteria belonging to the genus Bacillus include Bacillus subtilis MI114 (Gene, 24, 255 (1983)), 207-21 (Journal of Biochemistry, 95, 87 (1984)), etc.

Examples of yeast include Saccharomyces cereviseae AH22, AH22R⁻, NA87-11A, DKD-5D, 20B-12, Schizosaccharomyces pombe NCYC1913, NCYC2036, Pichia pastoris KM71, etc.

Examples of insect cells include, for the virus AcNPV, Spodoptera frugiperda cells (Sf cells), MG1 cells derived from mid-intestine of Trichoplusia ni, High Five™ cells derived from egg of Trichoplusia ni, cells derived from Mamestra brassicae, cells derived from Estigmena acrea, etc.; and for the virus BmNPV, Bombyx mori N cells (BmN cells), etc. are used. Examples of the Sf cell which can be used are Sf9 cells (ATCC CRL1711) and Sf21 cells (both cells are described in Vaughn, J. L. et al., In Vivo, 13, 213-217 (1977).

As the insect, for example, a larva of Bombyx mori can be used (Maeda, et al., Nature, 315, 592 (1985)).

Examples of animal cells include monkey cells COS-7, Vero, Chinese hamster cells CHO (hereinafter referred to as CHO cells), dhfr gene deficient Chinese hamster cells CHO (hereinafter simply referred to as CHO(dhfr⁻) cell), mouse L cells, mouse AtT-20, mouse myeloma cells, rat GH3, human FL cells, etc.

Bacteria belonging to the genus Escherichia can be transformed, for example, by the method described in Proc. Natl. Acad. Sci. U.S.A., 69, 2110 (1972) or Gene, 17, 107 (1982).

Bacteria belonging to the genus Bacillus can be transformed, for example, by the method described in Molecular & General Genetics, 168, 111 (1979).

Yeast can be transformed, for example, by the method described in Methods in Enzymology, 194, 182-187 (1991), Proc. Natl. Acad. Sci. U.S.A., 75, 1929 (1978), etc.

Insect cells or insects can be transformed, for example, according to the method described in Bio/Technology, 6, 47-55(1988), etc.

Animal cells can be transformed, for example, according to the method described in Saibo Kogaku (Cell Engineering), extra issue 8, Shiin Saibo Kogaku Jikken Protocol (New Cell Engineering Experimental Protocol), 263-267 (1995), published by Shujunsha, or Virology, 52, 456 (1973).

Thus, the transformant transformed with the expression vector containing the DNA encoding the protein can be obtained.

Where the host is bacteria belonging to the genus Escherichia or the genus Bacillus, the transformant can be appropriately incubated in a liquid medium which contains materials required for growth of the transformant such as carbon sources, nitrogen sources, inorganic materials, and so on. Examples of the carbon sources include glucose, dextrin, soluble starch, sucrose, etc. Examples of the nitrogen sources include inorganic or organic materials such as ammonium salts, nitrate salts, corn steep liquor, peptone, casein, meat extract, soybean cake, potato extract, etc. Examples of the inorganic materials are calcium chloride, sodium dihydrogenphosphate, magnesium chloride, etc. In addition, yeast extract, vitamins, growth promoting factors etc. may also be added to the medium. Preferably, pH of the medium is adjusted to about 5 to about 8.

A preferred example of the medium for incubation of the bacteria belonging to the genus Escherichia is M9 medium supplemented with glucose and Casamino acids (Miller, Journal of Experiments in Molecular Genetics, 431-433, Cold Spring Harbor Laboratory, New York, 1972). If necessary and desired, a chemical such as 3β-indolylacrylic acid can be added to the medium thereby to activate the promoter efficiently.

Where the bacteria belonging to the genus Escherichia are used as the host, the transformant is usually cultivated at about 15° C. to about 43° C. for about 3 hours to about 24 hours. If necessary and desired, the culture may be aerated or agitated.

Where the bacteria belonging to the genus Bacillus are used as the host, the transformant is cultivated generally at about 30° C. to about 40° C. for about 6 hours to about 24 hours. If necessary and desired, the culture can be aerated or agitated.

Where yeast is used as the host, the transformant is cultivated, for example, in Burkholder's minimal medium (Bostian, K. L. et al., Proc. Natl. Acad. Sci. U.S.A., 77, 4505 (1980)) or in SD medium supplemented with 0.5% Casamino acids (Bitter, G. A. et al., Proc. Natl. Acad. Sci. U.S.A., 81, 5330 (1984)). Preferably, pH of the medium is adjusted to about 5 to about 8. In general, the transformant is cultivated at about 20° C. to about 35° C. for about 24 hours to about 72 hours. If necessary and desired, the culture can be aerated or agitated.

Where insect cells or insects are used as the host, the transformant is cultivated in, for example, Grace's Insect Medium (Grace, T. C. C., Nature, 195, 788 (1962)) to which an appropriate additive such as immobilized 10% bovine serum is added. Preferably, pH of the medium is adjusted to about 6.2 to about 6.4. Normally, the transformant is cultivated at about 27° C. for about 3 days to about 5 days and, if necessary and desired, the culture can be aerated or agitated.

Where animal cells are employed as the host, the transformant is cultivated in, for example, MEM medium containing about 5% to about 20% fetal bovine serum (Science, 122, 501 (1952)), DMEM medium (Virology, 8, 396 (1959)), RPMI 1640 medium (The Journal of the American Medical Association, 199, 519 (1967)), 199 medium (Proceeding of the Society for the Biological Medicine, 73, 1 (1950)), etc. Preferably, pH of the medium is adjusted to about 6 to about 8. The transformant is usually cultivated at about 30° C. to about 40° C. for about 15 hours to about 60 hours and, if necessary and desired, the culture can be aerated or agitated.

As described above, the protein of the present invention can be produced into the cell, in the cell membrane or out of the cell of the transformant.

The protein of the present invention can be separated and purified from the culture described above by the following procedures.

When the protein of the present invention is extracted from the culture or cells, after cultivation the transformants or cells are collected by a publicly known method and suspended in a appropriate buffer. The transformants or cells are then disrupted by publicly known methods such as ultrasonication, a treatment with lysozyme and/or freeze-thaw cycling, followed by centrifugation, filtration, etc. Thus, the crude extract of the protein of the present invention can be obtained. The buffer used for the procedures may contain a protein modifier such as urea or guanidine hydrochloride, or a surfactant such as Triton X-100™, etc. When the protein is secreted in the culture, after completion of the cultivation the supernatant can be separated from the transformants or cells to collect the supernatant by a publicly known method.

The protein contained in the supernatant or the extract thus obtained can be purified by appropriately combining the publicly known methods for separation and purification. Such publicly known methods for separation and purification include a method utilizing difference in solubility such as salting out, solvent precipitation, etc.; a method utilizing mainly difference in molecular weight such as dialysis, ultrafiltration, gel filtration, SDS-polyacrylamide gel electrophoresis, etc.; a method utilizing difference in electric charge such as ion exchange chromatography, etc.; a method utilizing difference in specific affinity such as affinity chromatography, etc.; a method utilizing difference in hydrophobicity such as reverse phase high performance liquid chromatography, etc.; a method utilizing difference in isoelectric point such as isoelectrofocusing electrophoresis; and the like.

When the protein thus obtained is in a free form, it can be converted into the salt by publicly known methods or modifications thereof. On the other hand, when the protein is obtained in the form of a salt, it can be converted into the free form or in the form of a different salt by publicly known methods or modifications thereof.

The protein produced by the recombinant can be treated, prior to or after the purification, with an appropriate protein modifying enzyme so that the protein can be appropriately modified to partially remove a polypeptide. Examples of the protein-modifying enzyme include trypsin, chymotrypsin, arginyl endopeptidase, protein kinase, glycosidase or the like.

The activity of the thus produced protein of the present invention or salts thereof can be determined by a test binding to a labeled ligand, by an enzyme immunoassay using a specific antibody, or the like.

Antibodies to the protein of the present invention, its partial peptides, or salts thereof may be any of polyclonal antibodies and monoclonal antibodies, as long as they are capable of recognizing the protein of the present invention, its partial peptides, or salts thereof.

The antibodies to the protein of the present invention, its partial peptides, or salts thereof (hereinafter sometimes merely referred to as the protein of the present invention) may be manufactured by publicly known methods for manufacturing antibodies or antisera, using as antigens the protein of the present invention.

[Preparation of Monoclonal Antibody]

(a) Preparation of Monoclonal Antibody-Producing Cells

The protein of the present invention is administered to mammals either solely or together with carriers or diluents to the site where the production of antibody is possible by the administration. In order to potentiate the antibody productivity upon the administration, complete Freund's adjuvants or incomplete Freund's adjuvants may be administered. The administration is usually carried out once in every two to six weeks and 2 to 10 times in total. Examples of the applicable mammals are monkeys, rabbits, dogs, guinea pigs, mice, rats, sheep and goats, with mice and rats being preferred.

In the preparation of monoclonal antibody-producing cells, warm-blooded animals, e.g., mice, immunized with an antigen wherein the antibody titer is noted is selected, then the spleen or lymph node is collected after 2 to 5 days from the final immunization and antibody-producing cells contained therein are fused with myeloma cells to give monoclonal antibody-producing hybridomas. Measurement of the antibody titer in antisera may be made, for example, by reacting a labeled form of the protein, which will be described later, with the antiserum followed by assaying the binding activity of the labeling agent bound to the antibody. The fusion may be operated, for example, by the known Koehler and Milstein method (Nature, 256, 495, 1975). Examples of the fusion accelerator are polyethylene glycol (PEG), Sendai virus, etc., of which PEG is preferably employed.

Examples of the myeloma cells are NS-1, P3U1, SP2/0, AP-1, etc. In particular, P3U1 is preferably employed. A preferred ratio of the count of the antibody-producing cells used (spleen cells) to the count of myeloma cells is within a range of approximately 1:1 to 20:1. When PEG (preferably, PEG 1000 to PEG 6000) is added in a concentration of approximately 10 to 80% followed by incubating at about 20 to about 40° C., preferably at about 30 to about 37° C. for about 1 to about 10 minutes, an efficient cell fusion can be carried out.

Various methods can be used for screening of a monoclonal antibody-producing hybridoma. Examples of such methods include a method which comprises adding the supernatant of hybridoma to a solid phase (e.g., microplate) adsorbed with the protein as an antigen directly or together with a carrier, adding an anti-immunoglobulin antibody (when mouse cells are used for the cell fusion, anti-mouse immunoglobulin antibody is used) labeled with a radioactive substance or an enzyme, or Protein A and detecting the monoclonal antibody bound to the solid phase, and a method which comprises adding the supernatant of hybridoma to a solid phase adsorbed with an anti-immunoglobulin antibody or Protein A, adding the protein labeled with a radioactive substance or an enzyme and detecting the monoclonal antibody bound to the solid phase.

The monoclonal antibody can be selected by publicly known methods or by modifications of these methods. In general, the selection can be effected in a medium for animal cells supplemented with HAT (hypoxanthine, aminopterin and thymidine). Any selection and growth medium can be employed as far as the hybridoma can grow therein. For example, RPMI 1640 medium containing 1% to 20%, preferably 10% to 20% fetal bovine serum, GIT medium (Wako Pure Chemical Industries, Ltd.) containing 1% to 10% fetal bovine serum, a serum free medium for cultivation of a hybridoma (SFM-101, Nissui Seiyaku Co., Ltd.) and the like can be used for the selection and growth medium. The cultivation is carried out generally at 20° C. to 40° C., preferably at about 37° C., for 5 days to 3 weeks, preferably 1 to 2 weeks. The cultivation can be conducted normally in 5% CO₂. The antibody titer of the culture supernatant of hybridomas can be determined as in the assay for the antibody titer in antisera described above.

(b) Purification of Monoclonal Antibody

Separation and purification of a monoclonal antibody can be carried out by methods applied to conventional separation and purification of immunoglobulins, as in the conventional methods for separation and purification of polyclonal antibodies [e.g., salting-out, alcohol precipitation, isoelectric point precipitation, electrophoresis, adsorption and desorption with ion exchangers (e.g., DEAE), ultracentrifugation, gel filtration, or a specific purification method which comprises collecting only an antibody with an activated adsorbent such as an antigen-binding solid phase, Protein A, Protein G. etc. and dissociating the binding to obtain the antibody].

[Preparation of Polyclonal Antibody]

The polyclonal antibody of the present invention can be manufactured by publicly known methods or modifications thereof. For example, a complex of immunogen (antigen such as the protein of the present invention) and a carrier protein is prepared, and a mammal is immunized with the complex in a manner similar to the method described above for the manufacture of monoclonal antibodies. The product containing the antibody to the protein of the present invention is collected from the immunized animal followed by separation and purification of the antibody.

In the complex of an immunogen and a carrier protein used to immunize a mammal, the type of carrier protein and the mixing ratio of a carrier to hapten may be any type and in any ratio, as long as the antibody is efficiently produced to the hapten immunized by crosslinking to the carrier. For example, bovine serum albumin, bovine thyroglobulins, keyhole limpet hemocyanin, etc. is coupled to hapten in a carrier-to-hapten weight ratio of approximately 0.1 to 20, preferably about 1 to about 5.

A variety of condensing agents can be used for the coupling of a carrier to hapten. Glutaraldehyde, carbodiimide, maleimide activated ester, activated ester reagents containing thiol group or dithiopyridyl group, etc. are used for the coupling.

The condensation product is administered to warm-blooded animals either solely or together with carriers or diluents to the site in which the antibody can be produce by the administration. In order to potentiate the antibody productivity upon the administration, complete Freund's adjuvant or incomplete Freund's adjuvant may be administered. The administration is usually made once approximately in every 2 to 6 weeks and about 3 to about 10 times in total.

The polyclonal antibody can be collected from the blood, ascites, etc., preferably from the blood of mammals immunized by the method described above.

The polyclonal antibody titer in antiserum can be assayed by the same procedure as that for the determination of serum antibody titer described above. The separation and purification of the polyclonal antibody can be carried out, following the method for the separation and purification of immunoglobulin performed as applied to the separation and purification of monoclonal antibodies described hereinabove.

For an antisense nucleotide having a complementary or substantially complementary base sequence to that of the DNA encoding the protein or its partial peptide of the present invention, or a portion thereof (hereinafter, in the description about the antisense nucleotide, it is merely abbreviated these DNAs as the DNA of the present invention), any antisense nucleotides may be used, as long as it has a complementary or substantially complementary base sequence to that of the DNA of the present invention, or a portion thereof, and has a function suppressing an expression of the DNA. Above all, antisense DNA is preferred.

Examples of the substantially complementary base sequence to that of the DNA of the present invention include the base sequences having at least about 70% homology, preferably at least about 80% homology, more preferably at least about 90% homology, and most preferably at least about 95% homology to the entire or partial base sequence of the complementary base sequence of the DNA of the present invention (i.e., the complementary strand of the DNA of the present invention). In particular, among the entire base sequence of the complementary strand of the DNA of the present invention, preferred is an antisense nucleotide having at least about 70% homology, preferably at least about 80% homology, more preferably at least about 90% homology, and most preferably at least about 95% homology to the complementary strand of the base sequence encoding a portion of the N-terminal region of the protein of the present invention (e.g., the base sequence adjacent to initiation codon).

Specifically, it includes:
(1) An antisense nucleotide having a complementary or substantially complementary base sequence to that of the DNA having the base sequence represented by SEQ ID NO: 2 or SEQ ID NO: 7, or a portion thereof, preferably an antisense nucleotide having a complementary base sequence to that of the DNA having the base sequence represented by SEQ ID NO: 2 or SEQ ID NO: 7, or a portion thereof;
(2) An antisense nucleotide having a complementary or substantially complementary base sequence to that of the DNA having the base sequence represented by SEQ ID NO: 9 or SEQ ID NO: 10, or a portion thereof, preferably an antisense nucleotide having a complementary base sequence to that of the DNA having the base sequence represented by SEQ ID NO: 9 or SEQ ID NO: 10, or a portion thereof;
(3) An antisense nucleotide having a complementary or substantially complementary base sequence to that of the DNA having the base sequence represented by SEQ ID NO: 12 or SEQ ID NO: 13, or a portion thereof, preferably an antisense nucleotide having a complementary base sequence to that of the DNA having the base sequence represented by SEQ ID NO: 12 or SEQ ID NO: 13, or a portion thereof,
(4) An antisense nucleotide having a complementary or substantially complementary base sequence to that of the DNA having the base sequence represented by SEQ ID NO: 16 or SEQ ID NO: 17, or a portion thereof, preferably an antisense nucleotide having a complementary base sequence to that of the DNA having the base sequence represented by SEQ ID NO: 16 or SEQ ID NO: 17, or a portion thereof;
(5) An antisense nucleotide having a complementary or substantially complementary base sequence to that of the DNA having the base sequence represented by SEQ ID NO: 19 or SEQ ID NO: 22, or a portion thereof, preferably an antisense nucleotide having a complementary base sequence to that of the DNA having the base sequence represented by SEQ ID NO: 19 or SEQ ID NO: 22, or a portion thereof;
(6) An antisense nucleotide having a complementary or substantially complementary base sequence to that of the DNA having the base sequence represented by SEQ ID NO: 24 or SEQ ID NO: 25, or a portion thereof, preferably an antisense nucleotide having a complementary base sequence to that of the DNA having the base sequence represented by SEQ ID NO: 24 or SEQ ID NO: 25, or a portion thereof;
(7) An antisense nucleotide having a complementary or substantially complementary base sequence to that of the DNA having the base sequence represented by SEQ ID NO: 27 or SEQ ID NO: 28, or a portion thereof, preferably an antisense nucleotide having a complementary base sequence to that of the DNA having the base sequence represented by SEQ ID NO: 27 or SEQ ID NO: 28, or a portion thereof; and the like.

The antisense nucleotide comprises usually about 10 to 40 and preferably 15 to 30 bases.

To prevent degradation by hydrolase such as nuclease, a phosphate residue (phosphate) of each nucleotide constituting the antisense nucleotide may be, for example, substituted through chemical modification by the phosphate residue such as phosphorothioate, methylphosphonate, phosphorodithionate, etc. These antisense nucleotides can be manufactured by using a publicly known DNA synthesizer.

According to the present invention, antisense polynucleotides (nucleic acids) that can inhibit the replication or expression of the gene encoding the protein of the present invention can be designed and synthesized based on the base sequence information of the cloned or determined DNA encoding the protein. Such a polynucleotide (nucleic acid) is capable of hybridizing to RNA of the gene encoding the protein of the present invention to inhibit the synthesis or function of said RNA or capable of modulating or controlling the expression of the gene encoding the protein of the present invention via interaction with the protein-associated RNA. Polynucleotides complementary to the selected sequences of RNA associated with the protein of the present invention and polynucleotides specifically hybridizable to the protein-associated RNA are useful in modulating or controlling the expression of the gene encoding the protein of the present invention in vivo and in vitro, and useful for the treatment or diagnosis of diseases. The term “corresponding” is used to mean homologous to or complementary to a particular sequence of the nucleotide, base sequence or nucleic acid including the gene. The term “corresponding” between nucleotides, base sequences or nucleic acids and peptides (proteins) usually refer to amino acids of a peptide (protein) under the order derived from the sequence of nucleotides (nucleic acids) or their complements. In the gene encoding the protein of the present invention, the 5′ end hairpin loop, 5′ end 6-base-pair repeats, 5′ end untranslated region, polypeptide translation initiation codon, protein coding region, ORF translation termination codon, 3′ end untranslated region, 3′ end palindrome region, and 3′ end hairpin loop, may be selected as preferred target regions, though any other region may be selected as a target in the gene encoding the protein.

The relationship between the targeted nucleic acids and the polynucleotides complementary to at least a part of the target, that is, the relationship between the target and the polynucleotides hybridizable to the target, can be denoted to be “antisense”. Examples of the antisense polynucleotides include polynucleotides containing 2-deoxy-D-ribose, polynucleotides containing D-ribose, any other type of polynucleotides which are N-glycosides of a purine or pyrimidine base, or other polymers containing non-nucleotide backbones (e.g., protein nucleic acids and synthetic sequence-specific nucleic acid polymers commercially available) or other polymers containing nonstandard linkages (provided that the polymers contain nucleotides having such a configuration that allows base pairing or base stacking, as is found in DNA or RNA), etc. The antisense polynucleotides may be double-stranded DNA, single-stranded DNA, single-stranded RNA or a DNA:RNA hybrid, and may further include unmodified polynucleotides (or unmodified oligonucleotides), those with publicly known types of modifications, for example, those with labels known in the art, those with caps, methylated polynucleotides, those with substitution of one or more naturally occurring nucleotides by their analogue, those with intramolecular modifications of nucleotides such as those with uncharged linkages (e.g., methyl phosphonates, phosphotriesters, phosphoramidates, carbamates, etc.) and those with charged linkages or sulfur-containing linkages (e.g., phosphorothioates, phosphorodithioates, etc.), those having side chain groups such as proteins (nucleases, nuclease inhibitors, toxins, antibodies, signal peptides, poly-L-lysine, etc.), saccharides (e.g., monosaccharides, etc.), those with intercalators (e.g., acridine, psoralen, etc.), those containing chelators (e.g., metals, radioactive metals, boron, oxidative metals, etc.), those containing alkylating agents, those with modified linkages (e.g., a anomeric nucleic acids, etc.), and the like. Herein the terms “nucleoside”, “nucleotide” and “nucleic acid” are used to refer to moieties that contain not only the purine and pyrimidine bases, but also other heterocyclic bases, which have been modified. Such modifications may include methylated purines and pyrimidines, acylated purines and pyrimidines and other heterocyclic rings. Modified nucleotides and modified nucleotides also include modifications on the sugar moiety, wherein, for example, one or more hydroxyl groups may optionally be substituted with a halogen atom(s), an aliphatic group(s), etc., or may be converted into the corresponding functional groups such as ethers, amines, or the like.

The antisense polynucleotide (nucleic acid) of the present invention is RNA, DNA or a modified nucleic acid (RNA, DNA). Specific examples of the modified nucleic acid are, but not limited to, sulfur and thiophosphate derivatives of nucleic acids and those resistant to degradation of polynucleoside amides or oligonucleoside amides. The antisense nucleic acids of the present invention can be modified preferably based on the following design, that is, by increasing the intracellular stability of the antisense nucleic acid, increasing the cellular permeability of the antisense nucleic acid, increasing the affinity of the nucleic acid to the targeted sense strand to a higher level, or minimizing the toxicity, if any, of the antisense nucleic acid.

Many of such modifications are known in the art, as disclosed in J. Kawakami, et al., Pharm. Tech. Japan, Vol. 8, pp. 247, 1992; Vol. 8, pp. 395, 1992; S. T. Crooke, et al. ed., Antisense Research and Applications, CRC Press, 1993; etc.

The antisense nucleic acid of the present invention may contain altered or modified sugars, bases or linkages. The antisense nucleic acid may also be provided in a specialized form such as liposomes, microspheres, or may be applied to gene therapy, or may be provided in combination with attached moieties. Such attached moieties include polycations such as polylysine that act as charge neutralizers of the phosphate backbone, or hydrophobic moieties such as lipids (e.g., phospholipids, cholesterols, etc.) that enhance the interaction with cell membranes or increase uptake of the nucleic acid. Preferred examples of the lipids to be attached are cholesterols or derivatives thereof (e.g., cholesteryl chloroformate, cholic acid, etc.). These moieties may be attached to the nucleic acid at the 3′ or 5′ ends thereof and may also be attached thereto through a base, sugar, or intramolecular nucleoside linkage. Other moieties may be capping groups specifically placed at the 3′ or 5′ ends of the nucleic acid to prevent degradation by nucleases such as exonuclease, RNase, etc. Such capping groups include, but are not limited to, hydroxyl protecting groups known in the art, including glycols such as polyethylene glycol, tetraethylene glycol and the like.

The inhibitory action of the antisense nucleic acid can be examined using the transformant of the present invention, the gene expression system of the present invention in vivo and in vitro, or the translation system of the G protein-coupled receptor protein in vivo and in vitro. The nucleic acid can be applied to cells by a variety of publicly known methods.

Hereinafter, the protein of the present invention, its partial peptides, or salts thereof (hereinafter sometimes referred to as the protein of the present invention), the DNA encoding the protein of the present invention or its partial peptides (hereinafter sometimes referred to as the DNA of the present invention), the antibodies to the protein of the present invention (hereinafter sometimes referred to as the antibodies of the present invention), and the antisense nucleotides (hereinafter sometimes referred to as the antisense nucleotide of the present invention) are specifically described for the use or applications.

A pharmaceutical comprising a compound or a salt thereof that inhibits the activities of the protein of the present invention can be used by, for example, suppressing the transmission of K⁺ ion, as a prophylactic and/or therapeutic agent for alimentary diseases (e.g., irritable bowel syndrome, chronic ulcerative colitis, Crohn's disease, ischemic colitis, gastritis, peptic ulcer, proctitis, diarrhea, reflux esophagitis, duodenitis, etc.), reproductive system diseases (e.g., prostate hyperplasia, prostatitis, testis neurosis, ovarian cystoma, etc.), respiratory diseases (e.g., chronic obstructive pulmonary disease, asthma, etc.), central nervous system diseases (e.g., Alzheimer's disease, parkinsonian syndrome, schizophrenia, cerebrovascular dementia, cerebral ischemia, convulsions, etc.), diabetes mellitus, hypertension, circulatory diseases (e.g., heart failure, arrhythmia, long QT syndrome, arteriosclerosis, angina, etc.), pancreatic diseases (e.g., pancreatitis, hypopancreatism such as cystic fibrosis pancreas, etc.), liver diseases (e.g., cirrhosis hepatic, etc.), kidney diseases (e.g., kidney failure, uremia, etc.), cancer (e.g., lung cancer, kidney cancer, liver cancer, non-small cell lung cancer, ovarian cancer, prostate cancer, stomach cancer, bladder cancer, breast cancer, cancer of uterine cervix, colon cancer, rectum cancer, pancreatic cancer, tymoma, testicular tumor, esophageal cancer, myosarcoma, etc.), or the like. Preferred is a prophylactic and/or therapeutic agent for alimentary diseases, reproductive system diseases, respiratory diseases or the like.

A pharmaceutical comprising a compound or a salt thereof that enhances the activities of the protein of the present invention can be used by, for example, enhancing the transmission of K⁺ ion, as a prophylactic and/or therapeutic agent for alimentary diseases (e.g., irritable bowel syndrome, chronic ulcerative colitis, Crohn's disease, ischemic colitis, gastritis, peptic ulcer, proctitis, diarrhea, reflux esophagitis, duodenitis, etc.), reproductive system diseases (e.g., prostate hyperplasia, prostatitis, testis neurosis, ovarian cystoma, etc.), respiratory diseases (e.g., chronic obstructive pulmonary disease, asthma, etc.), central nervous system diseases (e.g., Alzheimer's disease, parkinsonian syndrome, schizophrenia, cerebrovascular dementia, cerebral ischemia, convulsions, etc.), diabetes mellitus, hypertension, circulatory diseases (e.g., heart failure, arrhythmia, long QT syndrome, arteriosclerosis, angina, etc.), pancreatic diseases (e.g., pancreatitis, hypopancreatism such as cystic fibrosis pancreas, etc.), liver diseases (e.g., cirrhosis hepatic, etc.), kidney diseases (e.g., kidney failure, uremia, etc.), cancer (e.g., lung cancer, kidney cancer, liver cancer, non-small cell lung cancer, ovarian cancer, prostate cancer, stomach cancer, bladder cancer, breast cancer, cancer of uterine cervix, colon cancer, rectum cancer, pancreatic cancer, tymoma, testicular tumor, esophageal cancer, myosarcoma, etc.), or the like. Preferred is a prophylactic and/or therapeutic agent for alimentary diseases, reproductive system diseases, respiratory diseases or the like.

### [1] A Prophylactic and/or Therapeutic Agent for Various Diseases Associated with the Protein of the Present Invention

The protein of the present invention possesses a transmission activity of K⁺ ion (e.g., an activity that detects a cell membrane potential or intracellular Ca²⁺ ion concentration and regulates the transmission of K+ ion) and the like, and thus plays an important role for regulation of cell excitement.

Therefore, when the DNA encoding the protein of the present invention has abnormality or deficiency, or when a reduced expression of the protein of the present invention is detected, various diseases such as alimentary diseases (e.g., irritable bowel syndrome, chronic ulcerative colitis, Crohn's disease, ischemic colitis, gastritis, peptic ulcer, proctitis, diarrhea, reflux esophagitis, duodenitis, etc.), reproductive system diseases (e.g., prostate hyperplasia, prostatitis, testis neurosis, ovarian cystoma, etc.), respiratory diseases (e.g., chronic obstructive pulmonary disease, asthma, etc.), central nervous system diseases (e.g., Alzheimer's disease, parkinsonian syndrome, schizophrenia, cerebrovascular dementia, cerebral ischemia, convulsions, etc.), diabetes mellitus, hypertension, circulatory diseases (e.g., heart failure, arrhythmia, long QT syndrome, arteriosclerosis, angina, etc.), pancreatic diseases (e.g., pancreatitis, hypopancreatism such as cystic fibrosis pancreas, etc.), liver diseases (e.g., cirrhosis hepatic, etc.), kidney diseases (e.g., kidney failure, uremia, etc.), cancer (e.g., lung cancer, kidney cancer, liver cancer, non-small cell lung cancer, ovarian cancer, prostate cancer, stomach cancer, bladder cancer, breast cancer, cancer of uterine cervix, colon cancer, rectum cancer, pancreatic cancer, tymoma, testicular tumor, esophageal cancer, myosarcoma, etc.), and the like, are developed.

Therefore, the protein of the present invention and the DNA of the present invention can be used for a pharmaceutical such as a prophylactic and/or therapeutic agent for alimentary diseases (e.g., irritable bowel syndrome, chronic ulcerative colitis, Crohn's disease, ischemic colitis, gastritis, peptic ulcer, proctitis, diarrhea, reflux esophagitis, duodenitis, etc.), reproductive system diseases (e.g., prostate hyperplasia, prostatitis, testis neurosis, ovarian cystoma, etc.), respiratory diseases (e.g., chronic obstructive pulmonary disease, asthma, etc.), central nervous system diseases (e.g., Alzheimer's disease, parkinsonian syndrome, schizophrenia, cerebrovascular dementia, cerebral ischemia, convulsions, etc.), diabetes mellitus, hypertension, circulatory diseases (e.g., heart failure, arrhythmia, long QT syndrome, arteriosclerosis, angina, etc.), pancreatic diseases (e.g., pancreatitis, hypopancreatism such as cystic fibrosis pancreas, etc.), liver diseases (e.g., cirrhosis hepatic, etc.), kidney diseases (e.g., kidney failure, uremia, etc.), cancer (e.g., lung cancer, kidney cancer, liver cancer, non-small cell lung cancer, ovarian cancer, prostate cancer, stomach cancer, bladder cancer, breast cancer, cancer of uterine cervix, colon cancer, rectum cancer, pancreatic cancer, tymoma, testicular tumor, esophageal cancer, myosarcoma, etc.), or the like.

When a patient has a reduced level of, or deficient of the protein, etc. of the present invention in his or her body and thus may not maintain homeostasis in the body or may not exhibit the transmission of K⁺ ion sufficiently or properly, the protein of the present invention can provide its role sufficiently or properly for the patient, (a) by administering the DNA of the present invention to the patient to express the protein of the present invention in the body, (b) by inserting the DNA of the present invention into a cell, expressing the protein of the present invention and then transplanting the cell to the patient, or (c) by administering the protein of the present invention to the patient, etc.

Where the DNA of the present invention is used as the prophylactic and/or therapeutic agents described above, the DNA per se is administered directly to warm-blooded animal; alternatively, the DNA is inserted into an appropriate vector such as retrovirus vector, adenovirus vector, adenovirus-associated virus vector, etc. and then administered to warm-blooded animal in a conventional manner. The DNA of the present invention may also be administered as intact DNA, or with adjuvants to assist its uptake by gene gun or through a catheter such as a catheter with a hydrogel.

Where the protein of the present invention is used as the aforesaid prophylactic and/or therapeutic agents, the protein is advantageously used on a purified level of at least 90%, preferably at least 95%, more preferably at least 98% and most preferably at least 99%.

The protein of the present invention can be used orally, for example, in the form of tablets which may be sugar coated if necessary, capsules, elixirs, microcapsules etc., or parenterally in the form of injectable preparations such as a sterile solution and a suspension in water or with other pharmaceutically acceptable liquid. These preparations can be manufactured by mixing the protein of the present invention with a physiologically acceptable known carrier, a flavoring agent, an excipient, a vehicle, an antiseptic agent, a stabilizer, a binder, etc. in a unit dosage form required in a generally accepted manner that is applied to making pharmaceutical preparations. The active ingredient in the preparation is controlled in such a dose that an appropriate dose is obtained within the specified range given.

Additives miscible with tablets, capsules, etc. include a binder such as gelatin, corn starch, tragacanth and gum arabic, an excipient such as crystalline cellulose, a swelling agent such as corn starch, gelatin and alginic acid, a lubricant such as magnesium stearate, a sweetening agent such as sucrose, lactose and saccharin, and a flavoring agent such as peppermint, akamono oil and cherry. When the unit dosage is in the form of capsules, liquid carriers such as oils and fats may further be used together with the additives described above. A sterile composition for injection may be formulated according to a conventional manner used to make pharmaceutical compositions, e.g., by dissolving or suspending the active ingredients in a vehicle such as water for injection with a naturally occurring vegetable oil such as sesame oil and coconut oil, etc. to prepare the pharmaceutical composition.

Examples of an aqueous medium for injection include physiological saline and an isotonic solution containing glucose and other auxiliary agents (e.g., D-sorbitol, D-mannitol, sodium chloride, etc.) and may be used in combination with an appropriate dissolution aid such as an alcohol (e.g., ethanol or the like), a polyalcohol (e.g., propylene glycol and polyethylene glycol), a nonionic surfactant (e.g., polysorbate 80™ and HCO-50), etc. Examples of the oily medium include sesame oil and soybean oil, which may also be used in combination with a dissolution aid such as benzyl benzoate and benzyl alcohol. The prophylactic/therapeutic agent described above may further be formulated with a buffer (e.g., phosphate buffer, sodium acetate buffer, etc.), a soothing agent (e.g., benzalkonium chloride, procaine hydrochloride, etc.), a stabilizer (e.g., human serum albumin, polyethylene glycol, etc.), a preservative (e.g., benzyl alcohol, phenol, etc.), an antioxidant, etc. The thus-prepared liquid for injection is normally filled in an appropriate ampoule.

The vector in which the DNA of the present invention has been inserted may also be prepared into pharmaceutical preparations in a manner similar to the procedures above. Such preparations are generally used parenterally.

Since the thus obtained pharmaceutical preparation is safe and low toxic, the preparation can be administered to warm-blooded animal (e.g., human, rat, mouse, guinea pig, rabbit, chicken, sheep, swine, bovine, horse, cat, dog, monkey, chimpanzee, etc.).

The dose of the protein of the present invention varies depending on target disease, subject to be administered, route for administration, etc.; for example, in oral administration for the treatment of irritable bowel syndrome, the dose is normally about 0.1 mg to about 100 mg, preferably about 1.0 to about 50 mg, and more preferably about 1.0 to about 20 mg per day for adult (as 60 kg body weight). In parenteral administration, the single dose varies depending on subject to be administered, target disease, etc. but it is advantageous for the treatment of irritable bowel syndrome to administer the active ingredient intravenously at a daily dose of about 0.01 to about 30 mg, preferably about 0.1 to about 20 mg, and more preferably about 0.1 to about 10 mg for adult (as 60 kg body weight). For other animal species, the corresponding dose as converted per 60 kg body weight can be administered.

### [2] A Screening of Candidate Compounds for Pharmaceutical Against Diseases

The protein of the present invention is useful for an agent for screening a compound that enhances or inhibits activities of the protein of the present invention, or a salt thereof.

The present invention provides (1) a method for screening a compound that enhances or inhibits activities of the protein of the present invention (e.g., transmission of K⁺ ion, etc.), or a salt thereof (hereinafter, sometimes referred to as an enhancer or an inhibitor), which comprises using the protein of the present invention. More specifically, for example, it provides (2) a method for screening an enhancer or an inhibitor, which comprises making a comparison between (i) transmission (activity) of K⁺ ion in the cells having an ability of producing the protein of the present invention and (ii) transmission (activity) of K⁺ ion in the mixture of the cells having an ability of producing the protein of the present invention and a test compound.

Specifically, the above-described method for screening comprises measuring a membrane potential accompanied by transmission of K⁺ ion using fluorescent dye in the case of (i) and (ii), and comparing as an index of transmission of K⁺ ion.

The test compounds include, for example, peptides, proteins, non-peptidic compounds, synthetic compounds, fermentation products, cell extracts, plant extracts, animal tissue extracts and the like. These compounds may be either novel compounds or publicly known compounds.

In order to carry out the above-described method for screening, first, the cells having an ability of producing the protein of the present invention is prepared by suspending in a buffer appropriate for the screening. For the buffer, any buffer that does not interfere with the transmission (activity) of K⁺ ion of the protein of the present invention is usable and examples of such a buffer are phosphate buffer, borate buffer, etc., having pH of 4 to 10 (preferably pH of 6 to 8).

For the cells having an ability of producing the protein of the present invention, for example, a host transformed with a vector comprising the DNA encoding the aforementioned protein of the present invention (a transformant) may be utilized. For the host, for example, animal cells such as CHO cells, etc. are preferably used. For the screening, a transformant, wherein the protein of the present invention will be expressed on the cell membrane by culturing with the method described above, is preferably used.

The transmission of K⁺ ion of the protein of the present invention can be measured by a publicly known method, e.g., the method described in Receptors and Channels, Vol. 6, pp. 337-350, 1999, or by a modification thereof.

For example, when transmission of K+ ion in the above (ii) is enhanced at least about 10%, preferably at least about 30% and more preferably at least about 50% to that the above (i), the test compound can be selected as a compound that enhances activities of the protein of the present invention, or a salt thereof.

In addition, when transmission of K⁺ ion in the above (ii) is inhibited (or suppressed) at least about 10%, preferably at least about 30% and more preferably at least about 50% to that the above (i), the test compound can be selected as a compound that inhibits activities of the protein of the present invention, or a salt thereof.

By screening a compound that activates or inhibits an enzyme activityin the case where genes such as secretion-type alkaline phosphatase, luciferase, etc. are inserted downstream the promoter for the gene encoding the protein of the present invention and expressed in various cells described above, and the test compound is brought into contact with the cells, the compound that enhances or inhibits the expression of the protein of the present invention, i.e., enhances or inhibits the activity of the protein of the present invention, or a salt thereof, can be screened.

The polynucleotide encoding the protein of the present invention is useful for a reagent for screening a compound that enhances or inhibits the expression of the gene encoding the protein of the present invention, or a salt thereof.

The present invention provides (3) a method for screening a compound that enhances or inhibits expression of the gene encoding the protein of the present invention, or a salt thereof (hereinafter, sometimes referred to as an enhancer or an inhibitor), which comprises using a polynucleotide encoding the protein of the present invention. More specifically, for example, it provides (4) a method for screening an enhancer or an inhibitor, which comprises making a comparison between (iii) the case where cells having an ability of producing the protein of the present invention are cultured and (iv) the case where a mixture of cells having an ability of producing the protein of the present invention and a test compound is cultured.

Specifically, the above-described method for screening comprises measuring an expression level of the gene encoding the protein of the present invention (specifically, an level of the protein of the present invention or a level of mRNA encoding the aforementioned protein) in the case of (iii) and (iv), and comparing.

The test compounds include, for example, peptides, proteins, non-peptidic compounds, synthetic compounds, fermentation products, cell extracts, plant extracts, animal tissue extracts and the like. These compounds may be either novel compounds or publicly known compounds.

In order to carry out the above-described method for screening, first, the cells having an ability of producing the protein of the present invention is prepared by suspending in a buffer appropriate for the screening. For the buffer, any buffer that does not interfere with the transmission (activity) of K⁺ ion of the protein of the present invention is usable and examples of such a buffer are phosphate buffer, borate buffer, etc., having pH of 4 to 10 (preferably pH of 6 to 8).

For the cells having an ability of producing the protein of the present invention, for example, a host transformed with a vector comprising the DNA encoding the aforementioned protein of the present invention (a transformant) may be utilized. For the host, for example, animal cells such as CHO cells, etc. are preferably used. For the screening, a transformant, wherein the protein of the present invention will be expressed on the cell membrane by culturing with the method described above, is preferably used.

The determination of the level of the protein of the present invention can be carried out by a publicly known method, e.g., measured the level of the protein of the present invention in the cell extracts, etc., using the antibody recognizing the protein of the present invention according to the method such as Western analysis and ELISA techniques, or a modification thereof.

The expression level of the gene encoding the protein of the present invention can be assayed according to the publicly known method, e.g., Northern blotting, Reverse transcription-polymerase chain reaction (RT-PCR) or Real time PCR analysis system (ABI, TaqMan polymerase chain reaction), or a modification thereof.

For example, when the expression level of the gene encoding the protein of the present invention in the above (ii) is enhanced at least about 10%, preferably at least about 30% and more preferably at least about 50% to that the above (i), the test compound can be selected as a compound that enhances the expression level of the gene encoding the protein of the present invention, or a salt thereof.

In addition, when the expression level of the gene encoding the protein of the present invention in the above (ii) is inhibited at least about 10%, preferably at least about 30% and more preferably at least about 50% to that the above (i), the test compound can be selected as a compound that inhibits the expression level of the gene encoding the protein of the present invention, or a salt thereof.

Further, the antibody of the present invention is useful for a reagent for screening a compound that enhances or inhibits the expression of the protein of the present invention, or a salt thereof.

The present invention provides (5) a method for screening a compound that enhances or inhibits expression of the protein of the present invention, or a salt thereof (hereinafter, sometimes referred to as an enhancer or an inhibitor), which comprises using an antibody of the present invention. More specifically, for example, it provides (6) a method for screening an enhancer or an inhibitor, which comprises making a comparison between (v) the case where cells having an ability of producing the protein of the present invention are cultured and (vi) the case where a mixture of cells having an ability of producing the protein of the present invention and a test compound is cultured.

Specifically, the above-described method for screening comprises measuring an expression level of the protein of the present invention (specifically, an level of the protein of the present invention) in the case of (v) and (vi), e.g., detecting the expression of the protein of the present invention, or quantifying the expression level of the protein of the present invention, and comparing.

The test compounds include, for example, peptides, proteins, non-peptidic compounds, synthetic compounds, fermentation products, cell extracts, plant extracts, animal tissue extracts and the like. These compounds may be either novel compounds or publicly known compounds.

In order to carry out the above-described method for screening, first, the cells having an ability of producing the protein of the present invention is prepared by suspending in a buffer appropriate for the screening. For the buffer, any buffer that does not interfere with the transmission (activity) of K⁺ ion of the protein of the present invention is usable and examples of such a buffer are phosphate buffer, borate buffer, etc., having pH of 4 to 10 (preferably pH of 6 to 8).

For the cells having an ability of producing the protein of the present invention, for example, a host transformed with a vector comprising the DNA encoding the aforementioned protein of the present invention (a transformant) may be utilized. For the host, for example, animal cells such as CHO cells, etc. are preferably used. For the screening, a transformant, wherein the protein of the present invention will be expressed on the cell membrane by culturing with the method described above, is preferably used.

The determination of the level of the protein of the present invention can be carried out by a publicly known method, e.g., measured the level of the protein of the present invention in the cell extracts, etc., using the antibody recognizing the protein of the present invention according to the method such as Western analysis and ELISA techniques, or a modification thereof.

For example, when the expression level of the protein of the present invention in the above (vi) is enhanced at least about 10%, preferably at least about 30% and more preferably at least about 50% to that the above (v), the test compound can be selected as a compound that enhances the expression level of the protein of the present invention, or a salt thereof.

For example, when the expression level of the protein of the present invention in the above (vi) is inhibited at least about 10%, preferably at least about 30% and more preferably at least about 50% to that the above (v), the test compound can be selected as a compound that inhibits the expression level of the protein of the present invention, or a salt thereof.

A kit for screening of the present invention comprises the protein of the present invention, its partial peptide or salts thereof, or cells having an ability of producing the protein of the present invention or a partial peptide thereof.

A compound or a salt thereof obtained by using the screening method or the screening kit of the present invention is the above-described test compound, e.g., a compound or a salt thereof, which is selected from peptide, protein, non-peptidic compound, synthetic compound, fermentation product, cell extract, plant extract, animal tissue extract, blood plasma and the like. In addition, it is also a compound that enhances or inhibited the activity of the protein of the present invention (e.g., transmission of K⁺ ion), or a salt thereof.

As a salt form of the compound, it is usable for the same as the salt form of the protein of the present invention described above.

The compound that enhances activities of the protein of the present invention, or a salt thereof is useful as a pharmaceutical such as a prophylactic and/or therapeutic agent for alimentary diseases (e.g., irritable bowel syndrome, chronic ulcerative colitis, Crohn's disease, ischemic colitis, gastritis, peptic ulcer, proctitis, diarrhea, reflux esophagitis, duodenitis, etc.), reproductive system diseases (e.g., prostate hyperplasia, prostatitis, testis neurosis, ovarian cystoma, etc.), respiratory diseases (e.g., chronic obstructive pulmonary disease,.asthma, etc.), central nervous system diseases (e.g., Alzheimer's disease, parkinsonian syndrome, schizophrenia, cerebrovascular dementia, cerebral ischemia, convulsions, etc.), diabetes mellitus, hypertension, circulatory diseases (e.g., heart failure, arrhythmia, long QT syndrome, arteriosclerosis, angina, etc.), pancreatic diseases (e.g., pancreatitis, hypopancreatism such as cystic fibrosis pancreas, etc.), liver diseases (e.g., cirrhosis hepatic, etc.), kidney diseases (e.g., kidney failure, uremia, etc.), cancer (e.g., lung cancer, kidney cancer, liver cancer, non-small cell lung cancer, ovarian cancer, prostate cancer, stomach cancer, bladder cancer, breast cancer, cancer of uterine cervix, colon cancer, rectum cancer, pancreatic cancer, tymoma, testicular tumor, esophageal cancer, myosarcoma, etc.), or the like.

The compound that inhibits activities of the protein of the present invention, or a salt thereof is useful as a pharmaceutical such as a prophylactic and/or therapeutic agent for alimentary diseases (e.g., irritable bowel syndrome, chronic ulcerative colitis, Crohn's disease, ischemic colitis, gastritis, peptic ulcer, proctitis, diarrhea, reflux esophagitis, duodenitis, etc.), reproductive system diseases (e.g., prostate hyperplasia, prostatitis, testis neurosis, ovarian cystoma, etc.), respiratory diseases (e.g., chronic obstructive pulmonary disease, asthma, etc.), central nervous system diseases (e.g., Alzheimer's disease, parkinsonian syndrome, schizophrenia, cerebrovascular dementia, cerebral ischemia, convulsions, etc.), diabetes mellitus, hypertension, circulatory diseases (e.g., heart failure, arrhythmia, long QT syndrome, arteriosclerosis, angina, etc.), pancreatic diseases (e.g., pancreatitis, hypopancreatism such as cystic fibrosis pancreas, etc.), liver diseases (e.g., cirrhosis hepatic, etc.), kidney diseases (e.g., kidney failure, uremia, etc.), cancer (e.g., lung cancer, kidney cancer, liver cancer, non-small cell lung cancer, ovarian cancer, prostate cancer, stomach cancer, bladder cancer, breast cancer, cancer of uterine cervix, colon cancer, rectum cancer, pancreatic cancer, tymoma, testicular tumor, esophageal cancer, myosarcoma, etc.), or the like.

The compound that enhances expression of the gene encoding the protein of the present invention, or a salt thereof is useful as a pharmaceutical such as a prophylactic and/or therapeutic agent for alimentary diseases (e.g., irritable bowel syndrome, chronic ulcerative colitis, Crohn's disease, ischemic colitis, gastritis, peptic ulcer, proctitis, diarrhea, reflux esophagitis, duodenitis, etc.), reproductive system diseases (e.g., prostate hyperplasia, prostatitis, testis neurosis, ovarian cystoma, etc.), respiratory diseases (e.g., chronic obstructive pulmonary disease, asthma, etc.), central nervous system diseases (e.g., Alzheimer's disease, parkinsonian syndrome, schizophrenia, cerebrovascular dementia, cerebral ischemia, convulsions, etc.), diabetes mellitus, hypertension, circulatory diseases (e.g., heart failure, arrhythmia, long QT syndrome, arteriosclerosis, angina, etc.), pancreatic diseases (e.g., pancreatitis, hypopancreatism such as cystic fibrosis pancreas, etc.), liver diseases (e.g., cirrhosis hepatic, etc.), kidney diseases (e.g., kidney failure, uremia, etc.), cancer (e.g., lung cancer, kidney cancer, liver cancer, non-small cell lung cancer, ovarian cancer, prostate cancer, stomach cancer, bladder cancer, breast cancer, cancer of uterine cervix, colon cancer, rectum cancer, pancreatic cancer, tymoma, testicular tumor, esophageal cancer, myosarcoma, etc.), or the like.

Further, the compound that inhibits expression of the gene encoding the protein of the present invention, or a salt thereof is useful as a pharmaceutical such as a prophylactic and/or therapeutic agent for alimentary diseases (e.g., irritable bowel syndrome, chronic ulcerative colitis, Crohn's disease, ischemic colitis, gastritis, peptic ulcer, proctitis, diarrhea, reflux esophagitis, duodenitis, etc.), reproductive system diseases (e.g., prostate hyperplasia, prostatitis, testis neurosis, ovarian cystoma, etc.), respiratory diseases (e.g., chronic obstructive pulmonary disease, asthma, etc.), central nervous system diseases (e.g., Alzheimer's disease, parkinsonian syndrome, schizophrenia, cerebrovascular dementia, cerebral ischemia, convulsions, etc.), diabetes mellitus, hypertension, circulatory diseases (e.g., heart failure, arrhythmia, long QT syndrome, arteriosclerosis, angina, etc.), pancreatic diseases (e.g., pancreatitis, hypopancreatism such as cystic fibrosis pancreas, etc.), liver diseases (e.g., cirrhosis hepatic, etc.), kidney diseases (e.g., kidney failure, uremia, etc.), cancer (e.g., lung cancer, kidney cancer, liver cancer, non-small cell lung cancer, ovarian cancer, prostate cancer, stomach cancer, bladder cancer, breast cancer, cancer of uterine cervix, colon cancer, rectum cancer, pancreatic cancer, tymoma, testicular tumor, esophageal cancer, myosarcoma, etc.), or the like.

The compound that enhances expression of the protein of the present invention, or a salt thereof is useful as a pharmaceutical such as a prophylactic and/or therapeutic agent for alimentary diseases (e.g., irritable bowel syndrome, chronic ulcerative colitis, Crohn's disease, ischemic colitis, gastritis, peptic ulcer, proctitis, diarrhea, reflux esophagitis, duodenitis, etc.), reproductive system diseases (e.g., prostate hyperplasia, prostatitis, testis neurosis, ovarian cystoma, etc.), respiratory diseases (e.g., chronic obstructive pulmonary disease, asthma, etc.), central nervous system diseases (e.g., Alzheimer's disease, parkinsonian syndrome, schizophrenia, cerebrovascular dementia, cerebral ischemia, convulsions, etc.), diabetes mellitus, hypertension, circulatory diseases (e.g., heart failure, arrhythmia, long QT syndrome, arteriosclerosis, angina, etc.), pancreatic diseases (e.g., pancreatitis, hypopancreatism such as cystic fibrosis pancreas, etc.), liver diseases (e.g., cirrhosis hepatic, etc.), kidney diseases (e.g., kidney failure, uremia, etc.), cancer (e.g., lung cancer, kidney cancer, liver cancer, non-small cell lung cancer, ovarian cancer, prostate cancer, stomach cancer, bladder cancer, breast cancer, cancer of uterine cervix, colon cancer, rectum cancer, pancreatic cancer, tymoma, testicular tumor, esophageal cancer, myosarcoma, etc.), or the like.

In addition, the compound that inhibits expression of the the protein of the present invention, or a salt thereof is useful as a pharmaceutical such as a prophylactic and/or therapeutic agent for alimentary diseases (e.g., irritable bowel syndrome, chronic ulcerative colitis, Crohn's disease, ischemic colitis, gastritis, peptic ulcer, proctitis, diarrhea, reflux esophagitis, duodenitis, etc.), reproductive system diseases (e.g., prostate hyperplasia, prostatitis, testis neurosis, ovarian cystoma, etc.), respiratory diseases (e.g., chronic obstructive pulmonary disease, asthma, etc.), central nervous system diseases (e.g., Alzheimer's disease, parkinsonian syndrome, schizophrenia, cerebrovascular dementia, cerebral ischemia, convulsions, etc.), diabetes mellitus, hypertension, circulatory diseases (e.g., heart failure, arrhythmia, long QT syndrome, arteriosclerosis, angina, etc.), pancreatic diseases (e.g., pancreatitis, hypopancreatism such as cystic-fibrosis pancreas, etc.), liver diseases (e.g., cirrhosis hepatic, etc.), kidney diseases (e.g., kidney failure, uremia, etc.), cancer (e.g., lung cancer, kidney cancer, liver cancer, non-small cell lung cancer, ovarian cancer, prostate cancer, stomach cancer, bladder cancer, breast cancer, cancer of uterine cervix, colon cancer, rectum cancer, pancreatic cancer, tymoma, testicular tumor, esophageal cancer, myosarcoma, etc.), or the like.

When the compound or a salt thereof obtained by using the screening method or the screening kit of the present invention is used as the above-described prophylactic and/or therapeutic agent, it may be prepared in a conventional manner, to tablets, capsules, elixirs, microcapsules, sterile solution or suspension.

Since the preparation thus obtained is safe and low toxic, it can be administered to human or other warm-blooded animals (e.g., mouse, rat, rabbit, sheep, swine, bovine, horse, fowl, cat, dog, monkey; chimpanzee, etc.).

Although the amount of the compound or its salt to be administered varies depending upon its function, target disease, subject to be administered, route of administration, etc., when the compound that enhances the activity of the protein of the present invention is orally administered, the compound is generally administered to an adult patient with irritable bowel syndrome (as 60 kg body weight) in a dose of about 0.1 mg/day to about 100 mg/day, preferably about 1.0 mg/day to about 50 mg/day, more preferably about 1.0 mg to about 20 mg. For parenteral administration, a single dose of the compound varies depending upon subject to be administered, target disease, etc., but when the compound is administered to an adult patient with irritable bowel syndrome (as 60 kg body weight) in the form of an injectable preparation, it is generally advantageous to administer the compound intravenously in a dose of about 0.01 mg/day to about 30 mg/day, preferably about 0.1 mg/day to about 20 mg/day, more preferably about 0.1 mg/day to about 10 mg/day. As for other animals, the compound can be administered in the above amount with converting it into that for the body weight of 60 kg.

### [3] Quantification of the Protein of the Present Invention, its Partial Peptide or Salts Thereof

The antibodies of the present invention are capable of specifically recognizing the protein of the present invention. Therefore, the antibodies can be used to quantify the protein of the present invention in a test fluid, especially for quantification by the sandwich immunoassay.

That is, the present invention provides, for example, the following quantification methods:
(i) A method of quantifying the protein of the present invention in a test fluid, which comprises competitively reacting the antibody of the present invention with the test fluid and a labeled form of the protein of the present invention, and measuring the ratio of the labeled protein of the present invention bound to the antibody; and
(ii) A method of quantifying the protein of the present invention in a test fluid, which comprises reacting the test fluid with the antibody of the present invention immobilized on a carrier and a labeled form of the antibody of the present invention simultaneously or sequentially, and measuring the activity of the label on the immobilized carrier.

In (ii) described above, it is preferred that one antibody recognizes the N-terminal region of the protein of the present invention, and another antibody reacts with the C-terminal region of the protein of the present invention.

Using monoclonal antibodies to the protein of the present invention (hereinafter sometimes referred to as the monoclonal antibodies of the present invention), the protein of the present invention can be assayed and also detected by tissue staining or the like. For this purpose, an antibody molecule itself may be used, or F(ab′)₂, Fab′ or Fab fractions of the antibody molecule may also be used. Assay methods using antibodies to the receptor protein etc. of the present invention are not particularly limited. Any assay method can be used, so long as the amount of antibody, antigen, or antibody-antigen complex corresponding to the amount of antigen (e.g., the amount of the receptor protein) in the test fluid can be detected by chemical or physical means and the amount of the antigen can be calculated from a standard curve prepared from standard solutions containing known amounts of the antigen. For example, nephrometry, competitive methods, immunometric method, and sandwich method are appropriately used, with the sandwich method described below being most preferable in terms of sensitivity and specificity.

As the labeling agent for the methods using labeled substances, there are employed, for example, radioisotopes, enzymes, fluorescent substances, luminescent substances, etc. For the radioisotope, for example, [¹²⁵I], [¹³¹I], [³H] and [¹⁴C] are used. As the enzyme described above, stable enzymes with high specific activity are preferred; for example, β-galactosidase, β-glucosidase, alkaline phosphatase, peroxidase, malate dehydrogenase and the like are used. Example of the fluorescent substance used is fluorescamine and fluorescein isothiocyanate are used. For the luminescent substance, for example, luminol, luminol derivatives, luciferin, and lucigenin can be used. Furthermore, the biotin-avidin system may be used for binding antibody or antigen to the label.

For immobilization of antigen or antibody, physical adsorption may be used. Chemical binding methods conventionally used for insolubilization or immobilization of proteins or enzymes may also be used. For the carrier, for example, insoluble polysaccharides such as agarose, dextran, cellulose, etc.; synthetic resin such as polystyrene, polyacrylamide, silicon, etc., and glass or the like are used.

In the sandwich method, the immobilized monoclonal antibody of the present invention is reacted with a test fluid (primary reaction), then with the labeled monoclonal antibody of the present invention (secondary reaction), and the activity of the label on the immobilizing carrier is measured, whereby the amount of the protein of the present invention in the test fluid can be quantified. The order of the primary and secondary reactions may be reversed, and the reactions may be performed simultaneously or with an interval. The methods of labeling and immobilization can be performed by the methods described above. In the immunoassay by the sandwich method, the antibody used for immobilized or labeled antibodies is not necessarily one species, but a mixture of two or more species of antibody may be used to increase the measurement sensitivity.

In the methods of assaying the protein of the present invention by the sandwich method, antibodies that bind to different sites of the protein are preferably used as the monoclonal antibodies of the present invention for the primary and secondary reactions. That is, in the antibodies used for the primary and secondary reactions are, for example, when the antibody used in the secondary reaction recognizes the C-terminal region of the protein, it is preferable to use the antibody recognizing the region other than the C-terminal region for the primary reaction, e.g., the antibody recognizing the N-terminal region.

The monoclonal antibodies of the present invention can be used for the assay systems other than the sandwich method, for example, competitive method, immunometric method, nephrometry, etc.

In the competitive method, antigen in a test fluid and the labeled antigen are competitively reacted with antibody, and the unreacted labeled antigen (F) and the labeled antigen bound to the antibody (B) are separated (B/F separation). The amount of the label in B or F is measured, and the amount of the antigen in the test fluid is quantified. This reaction method includes a liquid phase method using a soluble antibody as an antibody, polyethylene glycol for B/F separation and a secondary antibody to the soluble antibody, and an immobilized method either using an immobilized antibody as the primary antibody, or using a soluble antibody as the primary antibody and immobilized antibody as the secondary antibody.

In the immunometric method, antigen in a test fluid and immobilized antigen are competitively reacted with a definite amount of labeled antibody, the immobilized phase is separated from the liquid phase, or antigen in a test fluid and an excess amount of labeled antibody are reacted, immobilized antigen is then added to bind the unreacted labeled antibody to the immobilized phase, and the immobilized phase is separated from the liquid phase. Then, the amount of the label in either phase is measured to quantify the antigen in the test fluid.

In the nephrometry, insoluble precipitate produced after the antigen-antibody reaction in gel or solution is quantified. When the amount of antigen in the test fluid is small and only a small amount of precipitate is obtained, laser nephrometry using scattering of laser is advantageously employed.

For applying these immunological methods to the measurement methods of the present invention, any particular conditions or procedures are not required. Systems for measuring the protein of the present invention or its salts are constructed by adding the usual technical consideration in the art to the conventional conditions and procedures. For the details of these general technical means, reference can be made to the following reviews and texts. [For example, Hiroshi Irie, ed. “Radioimmunoassay” (Kodansha, published in 1974), Hiroshi Irie, ed. “Sequel to the Radioimmunoassay” (Kodansha, published in 1979), Eiji Ishikawa, et al. ed. “Enzyme immonoassay” (Igakushoin, published in 1978), Eiji Ishikawa, et al. ed. “Immunoenzyme assay” (2nd ed.) (Igakushoin, published in 1982), Eiji Ishikawa, et al. ed. “Immunoenzyme assay” (3rd ed.) (Igakushoin, published in 1987), Methods in ENZYMOLOGY, Vol. 70 (Immunochemical Techniques (Part A)), ibid., Vol. 73 (Immunochemical Techniques (Part B)), ibid., Vol. 74 (Immunochemical Techniques (Part C)), ibid., Vol. 84 (Immunochemical Techniques (Part D: Selected Immunoassays)), ibid., Vol. 92 (Immunochemical Techniques (Part E: Monoclonal Antibodies and General Immunoassay Methods)), ibid., Vol. 121 (Immunochemical Techniques (Part I: Hybridoma Technology and Monoclonal Antibodies))(all published by Academic Press Publishing).]

As described above, the protein of the present invention or its salts can be quantified with high sensitivity, using the antibodies of the present invention.

When the reduced concentration of the protein of the present invention was detected by quantifying the concentration of the protein of the present invention using the antibody of the present invention, it can be diagnosed that it is highly possible to develop, for example, alimentary diseases (e.g., irritable bowel syndrome, chronic ulcerative colitis, Crohn's disease, ischemic colitis, gastritis, peptic ulcer, proctitis, diarrhea, reflux esophagitis, duodenitis, etc.), reproductive system diseases (e.g., prostate hyperplasia, prostatitis, testis neurosis, ovarian cystoma, etc.), respiratory diseases (e.g., chronic obstructive pulmonary disease, asthma, etc.), central nervous system diseases (e.g., Alzheimer's disease, parkinsonian syndrome, schizophrenia, cerebrovascular dementia, cerebral ischemia, convulsions, etc.), diabetes mellitus, hypertension, circulatory diseases (e.g., heart failure, arrhythmia, long QT syndrome, arteriosclerosis, angina, etc.), pancreatic diseases (e.g., pancreatitis, hypopancreatism such as cystic fibrosis pancreas, etc.), liver diseases (e.g., cirrhosis hepatic, etc.), kidney diseases (e.g., kidney failure, uremia, etc.), cancer (e.g., lung cancer, kidney cancer, liver cancer, non-small cell lung cancer, ovarian cancer, prostate cancer, stomach cancer, bladder cancer, breast cancer, cancer of uterine cervix, colon cancer, rectum cancer, pancreatic cancer, tymoma, testicular tumor, esophageal cancer, myosarcoma, etc.), or the like. In addition, when the increased concentration of the protein of the present invention was detected, it can be diagnosed that it is highly possible to develop, for example, alimentary diseases (e.g., irritable bowel syndrome, chronic ulcerative colitis, Crohn's disease, ischemic colitis, gastritis, peptic ulcer, proctitis, diarrhea, reflux esophagitis, duodenitis, etc.), reproductive system diseases (e.g., prostate hyperplasia, prostatitis, testis neurosis, ovarian cystoma, etc.), respiratory diseases (e.g., chronic obstructive pulmonary disease, asthma, etc.), central nervous system diseases (e.g., Alzheimer's disease, parkinsonian syndrome, schizophrenia, cerebrovascular dementia, cerebral ischemia, convulsions, etc.), diabetes mellitus, hypertension, circulatory diseases (e.g., heart failure, arrhythmia, long QT syndrome, arteriosclerosis, angina, etc.), pancreatic diseases (e.g., pancreatitis, hypopancreatism such as cystic fibrosis pancreas, etc.), liver diseases (e.g., cirrhosis hepatic, etc.), kidney diseases (e.g., kidney failure, uremia, etc.), cancer (e.g., lung cancer, kidney cancer, liver cancer, non-small cell lung cancer, ovarian cancer, prostate cancer, stomach cancer, bladder cancer, breast cancer, cancer of uterine cervix, colon cancer, rectum cancer, pancreatic cancer, tymoma, testicular tumor, esophageal cancer, myosarcoma, etc.), or the like.

Further, the antibody of the present invention may be used for detecting the protein of the present invention existed in the test sample such as body fluid and tissues. Furthermore, it can be used for preparing an antibody column for purification of the protein of the present invention, detecting the protein of the present invention in each fraction of purification, analyzing a profile of the protein of the present invention in the cells tested, and the like.

### [4] Gene Diagnostic Agent

By using the DNA of the invention, e.g., as a probe, an abnormality (gene abnormality) of the DNA or mRNA encoding the protein or its partial peptide of the invention in human or other warm-blooded animal (e.g., rat, mouse, guinea pig, rabbit, chicken, sheep, swine, bovine, horse, cat, dog, monkey, chimpanzee, etc.) can be detected. Therefore, the DNA of the invention is useful as a gene diagnostic agent for damages to the DNA or mRNA, its mutation or decreased expression, or increased expression or overexpression of the DNA or mRNA.

The gene diagnosis described above using the DNA of the invention can be performed by, for example, publicly known Northern hybridization or PCR-SSCP assay (Genomics, 5, 874-879 (1989); Proceedings of the National Academy of Sciences of the United States of America, 86, 2766-2770 (1989)), etc.

For example, when increased expression is detected by northern hybridization, it can be diagnosed that it is highly likely to suffer from alimentary diseases (e.g., irritable bowel syndrome, chronic ulcerative colitis, Crohn's disease, ischemic colitis, gastritis, peptic ulcer, proctitis, diarrhea, reflux esophagitis, duodenitis, etc.), reproductive system diseases (e.g., prostate hyperplasia, prostatitis, testis neurosis, ovarian cystoma, etc.), respiratory diseases (e.g., chronic obstructive pulmonary disease, asthma, etc.), central nervous system diseases (e.g., Alzheimer's disease, parkinsonian syndrome, schizophrenia, cerebrovascular dementia, cerebral ischemia, convulsions, etc.), diabetes mellitus, hypertension, circulatory diseases (e.g., heart failure, arrhythmia, long QT syndrome, arteriosclerosis, angina, etc.), pancreatic diseases (e.g., pancreatitis, hypopancreatism such as cystic fibrosis pancreas, etc.), liver diseases (e.g., cirrhosis hepatic, etc.), kidney diseases (e.g., kidney failure, uremia, etc.), cancer (e.g., lung cancer, kidney cancer, liver cancer, non-small cell lung cancer, ovarian cancer, prostate cancer, stomach cancer, bladder cancer, breast cancer, cancer of uterine cervix, colon cancer, rectum cancer, pancreatic cancer, tymoma, testicular tumor, esophageal cancer, myosarcoma, etc.), or the like. On the contrary, when reduced expression is detected by northern hybridization or DNA microarray, or when DNA mutation is detected by PCR-SSCP, it can be diagnosed that it is highly likely to suffer from alimentary diseases (e.g., irritable bowel syndrome, chronic ulcerative colitis, Crohn's disease, ischemic colitis, gastritis, peptic ulcer, proctitis, diarrhea, reflux esophagitis, duodenitis, etc.), reproductive system diseases (e.g., prostate hyperplasia, prostatitis, testis neurosis, ovarian cystoma, etc.), respiratory diseases (e.g., chronic obstructive pulmonary disease, asthma, etc.), central nervous system diseases (e.g., Alzheimer's disease, parkinsonian syndrome, schizophrenia, cerebrovascular dementia, cerebral ischemia, convulsions, etc.), diabetes mellitus, hypertension, circulatory diseases (e.g., heart failure, arrhythmia, long QT syndrome, arteriosclerosis, angina, etc.), pancreatic diseases (e.g., pancreatitis, hypopancreatism such as cystic fibrosis pancreas, etc.), liver diseases (e.g., cirrhosis hepatic, etc.), kidney diseases (e.g., kidney failure, uremia, etc.), cancer (e.g., lung cancer, kidney cancer, liver cancer, non-small cell lung cancer, ovarian cancer, prostate cancer, stomach cancer, bladder cancer, breast cancer, cancer of uterine cervix, colon cancer, rectum cancer, pancreatic cancer, tymoma, testicular tumor, esophageal cancer, myosarcoma, etc.), or the like.

### [5] Pharmaceuticals Comprising Antisense Nucleotide

Since the antisense nucleotide of the present invention, which binds to the DNA of the invention complementarily and suppresses the DNA expression, is low toxic and can suppress in vivo the functions of the protein of the present invention or the DNA of the present invention (e.g., transmission of K⁺ ion), it can be used as the agent for the treatment/prevention of alimentary diseases (e.g., irritable bowel syndrome, chronic ulcerative colitis, Crohn's disease, ischemic colitis, gastritis, peptic ulcer, proctitis, diarrhea, reflux esophagitis, duodenitis, etc.), reproductive system diseases (e.g., prostate hyperplasia, prostatitis, testis neurosis, ovarian cystoma, etc.), respiratory diseases (e.g., chronic obstructive pulmonary disease, asthma, etc.), central nervous system diseases (e.g., Alzheimer's disease, parkinsonian syndrome, schizophrenia, cerebrovascular dementia, cerebral ischemia, convulsions, etc.), diabetes mellitus, hypertension, circulatory diseases (e.g., heart failure, arrhythmia, long QT syndrome, arteriosclerosis, angina, etc.), pancreatic diseases (e.g., pancreatitis, hypopancreatism such as cystic fibrosis pancreas, etc.), liver diseases (e.g., cirrhosis hepatic, etc.), kidney diseases (e.g., kidney failure, uremia, etc.), cancer (e.g., lung cancer, kidney cancer, liver cancer, non-small cell lung cancer, ovarian cancer, prostate cancer, stomach cancer, bladder cancer, breast cancer, cancer of uterine cervix, colon cancer, rectum cancer, pancreatic cancer, tymoma, testicular tumor, esophageal cancer, myosarcoma, etc.), or the like. Preferred is a preventive and/or therapeutic agent for alimentary diseases, reproductive system diseases, respiratory diseases and the like.

When the antisense nucleotide described above may be employed as the above prophylactic and/or therapeutic agents, it can be prepared according to the publicly known methods and can be administered.

For example, when the antisense nucleotide described above is used, it is administered solely, or it is inserted into an appropriate vector such as retrovirus vector, adenovirus vector, adenovirus-associated virus vector, etc., which is then administered orally or parenterally to human or other mammals (e.g., rat, rabbit, sheep, swine, bovine, cat, dog, monkey, etc.) in a conventional manner. The antisense nucleotide may be administered as it stands, or may be prepared into a dosage form together with a physiologically acceptable carrier to increase its uptake and administered by gene gun or through a catheter such as a catheter with a hydrogel.

Although the amount of the antisense nucleotide to be administered varies depending upon particular disease, subject to be administered, route of administration, etc., when the antisense nucleotide is locally administered to large intestine for prevention and/ or treatment of irritable bowel syndrome, the antisense nucleotide may be administered, in adult (60 kg of body weight), in a dose of about 0.1 mg/day to about 100 mg/day.

Further, the antisense nucleotide may also be employed as an oligonucleotide probe for diagnosis to examine the presence of the DNA of the invention in tissues or cells and the conditions of its expression.

The present invention further provides:
(i) A double-stranded RNA containing a part of the RNA encoding the protein of the present invention and its complementary RNA;
(ii) A pharmaceutical comprising the double-stranded RNA described above;
(iii) A ribozyme containing a part of the RNA encoding the protein of the present invention;
(iv) A pharmaceutical comprising the ribozyme described above;
(v) An expression vector containing the gene (the DNA) encoding the ribozyme described above; and the like.

As in the antisense nucleotide described above, the double-stranded RNA, ribozyme, etc. can destroy the RNA transcribed from the DNA of the present invention or suppress the functions of the RNA. Further, they can suppress the in vivo function of the protein or the DNA of the invention. Therefore, they can be used as preventive/therapeutic agents for alimentary diseases (e.g., irritable bowel syndrome, chronic ulcerative colitis, Crohn's disease, ischemic colitis, gastritis, peptic ulcer, proctitis, diarrhea, reflux esophagitis, duodenitis, etc.), reproductive system diseases (e.g., prostate hyperplasia, prostatitis, testis neurosis, ovarian cystoma, etc.), respiratory diseases (e.g., chronic obstructive pulmonary disease, asthma, etc.), central nervous system diseases (e.g., Alzheimer's disease, parkinsonian syndrome, schizophrenia, cerebrovascular dementia, cerebral ischemia, convulsions, etc.), diabetes mellitus, hypertension, circulatory diseases (e.g., heart failure, arrhythmia, long QT syndrome, arteriosclerosis, angina, etc.), pancreatic diseases (e.g., pancreatitis, hypopancreatism such as cystic fibrosis pancreas, etc.), liver diseases (e.g., cirrhosis hepatic, etc.), kidney diseases (e.g., kidney failure, uremia, etc.), cancer (e.g., lung cancer, kidney cancer, liver cancer, non-small cell lung cancer, ovarian cancer, prostate cancer, stomach cancer, bladder cancer, breast cancer, cancer of uterine cervix, colon cancer, rectum cancer, pancreatic cancer, tymoma, testicular tumor, esophageal cancer, myosarcoma, etc.), or the like. Preferred is a prophylactic and/or therapeutic agent for alimentary diseases, reproductive system diseases, respiratory diseases and the like.

The double-stranded RNA can be manufactured by designing the same based on the sequence of the polynucleotide of the present invention, by publicly known methods (e.g., Nature, 411, 494, 2001) with a modification.

The ribozyme can be manufactured by designing the same based on the sequence of the polynucleotide of the present invention, by a modification of publicly known methods (e.g., TRENDS in Molecular Medicine, 7, 221, 2001). For example, the ribozyme can be manufactured by replacing a partial sequence of the publicly known ribozyme with a part of the RNA encoding the protein of the present invention. The part of the RNA encoding the protein of the present invention includes a contiguous part to the consensus sequence NUX (in the sequence, N means all bases and X means bases except for G), which can be cleaved by a publicly known ribozyme.

Where the double-stranded RNA or ribozyme described above is used as the prophylactic and/or therapeutic agent described above, the RNA or ribozyme may be prepared into pharmaceutical preparations, as in the antisense polynucleotide, which are provided for administration. In addition, the expression vector described in (v) above, may be used as the prophylactic and/or therapeutic agent, just like the publicly known gene therapy.

### [6] Pharmaceuticals Comprising the Antibody of the Present Invention

The antibody of the present invention, which possesses a neutralizing activity to the activity of the protein of the present invention, can be used as a prophylactic and/or therapeutic agent for alimentary diseases (e.g., irritable bowel syndrome, chronic ulcerative colitis, Crohn's disease, ischemic colitis, gastritis, peptic ulcer, proctitis, diarrhea, reflux esophagitis, duodenitis, etc.), reproductive system diseases (e.g., prostate hyperplasia, prostatitis, testis neurosis, ovarian cystoma, etc.), respiratory diseases (e.g., chronic obstructive pulmonary disease, asthma, etc.), central nervous system diseases (e.g., Alzheimer's disease, parkinsonian syndrome, schizophrenia, cerebrovascular dementia, cerebral ischemia, convulsions, etc.), diabetes mellitus, hypertension, circulatory diseases (e.g., heart failure, arrhythmia, long QT syndrome, arteriosclerosis, angina, etc.), pancreatic diseases (e.g., pancreatitis, hypopancreatism such as cystic fibrosis pancreas, etc.), liver diseases (e.g., cirrhosis hepatic, etc.), kidney diseases (e.g., kidney failure, uremia, etc.), cancer (e.g., lung cancer, kidney cancer, liver cancer, non-small cell lung cancer, ovarian cancer, prostate cancer, stomach cancer, bladder cancer, breast cancer, cancer of uterine cervix, colon cancer, rectum cancer, pancreatic cancer, tymoma, testicular tumor, esophageal cancer, myosarcoma, etc.), and the like. Preferred are prophylactic and/or therapeutic agents for alimentary diseases, reproductive system diseases, respiratory diseases and the like.

Since the prophylactic and/or therapeutic agent for the above-mentioned diseases comprising the antibody of the present invention is low toxic, it can orally or parenterally (e.g., by intravenous injection) be administered to human or other mammals (e.g., rat, rabbit, sheep, swine, bovine, cat, dog, monkey) as a liquid formulation or an appropriate dosage form of pharmaceutical compositions. Although the amount of the antibody to be administered varies depending upon particular disease, subject to be administered, route of administration, etc., when the antibody is used for prevention and/or treatment of an adult patient with irritable bowel syndrome, it is generally advantageous to administer the antibody intravenously in a dose of about 0.01 mg/kg of body weight to about 20 mg/kg of body weight, preferably about 0.1 mg/kg of body weight to about 10 mg/kg of body weight, further preferably about 0.1 mg/kg of body weight to about 5 mg/kg of body weight. In other parenteral or oral administration, an amount based on the aforementioned amount may be administered. When symptom is particularly severe, the amount can be increased depending on the symptom.

The antibody of the present invention may be administered as it is or as an appropriate pharmaceutical composition. The pharmaceutical composition used in the administration described above comprises the aforementioned antibody or a salt thereof, and a pharmacologically acceptable carrier, diluent or excipient. Such composition is provided as a dosage form suitable for oral or parenteral administration.

In addition, each composition described above may comprise other active ingredients unless undesirable interactions occurs by blending with the antibody.

### [7] Preparation of an Animal having the DNA of the Present Invention

The present invention provides a non-human mammal bearing DNA encoding the protein of the present invention, which is exogenous (hereinafter abbreviated as the exogenous DNA of the present invention) or its variant DNA (sometimes simply referred to as the exogenous variant DNA of the present invention).

Thus, the present invention provides:
(1) A non-human mammal bearing the exogenous DNA or its variant DNA;
(2) The mammal according to (1), wherein the non-human mammal is a rodent;
(3) The mammal according to (2), wherein the rodent is mouse or rat; and,
(4) A recombinant vector bearing the exogenous DNA of the present invention or its variant DNA and capable of expressing in a mammal.

The non-human mammal bearing the exogenous DNA of the present invention or its variant DNA (hereinafter simply referred to as the DNA transgenic animal of the present invention) can be prepared by transfecting a desired DNA into an unfertilized egg, a fertilized egg, a spermatozoon, a germinal cell containing a primordial germinal cell thereof, or the like, preferably in the embryogenic stage in the development of a non-human mammal (more preferably in the single cell or fertilized cell stage and generally before the 8-cell phase), by standard means, such as the calcium phosphate method, the electric pulse method, the lipofection method, the agglutination method, the microinjection method, the particle gun method, the DEAE-dextran method etc. In addition, it is possible to transfect the exogenous DNA of the present invention into a somatic cell, a living organ, a tissue cell, or the like by the DNA transfection methods, and utilize the transformant for cell culture, tissue culture, etc. Further, these cells may be fused with the above-described germinal cell by a publicly known cell fusion method to prepare the transgenic animal of the present invention.

Examples of the non-human mammal that can be used include bovine, swine, sheep, goat, rabbits, dogs, cats, guinea pigs, hamsters, mice, rats and the like. Above all, preferred are rodents, especially mice (e.g., C57B1/6 strain, DBA2 strain, etc. for a pure line and for a cross line, B6C3F, strain, BDF, strain B6D2F, strain, BALB/c strain, ICR strain, etc.) or rats (Wistar, SD, etc.), since they are relatively short in ontogeny and life cycle from a standpoint of preparing model animals for human disease.

“Mammals” in a recombinant vector that can be expressed in the mammals include the aforesaid non-human mammals and human.

The exogenous DNA of the present invention refers to the DNA of the present invention that is once isolated and extracted from mammals, not the DNA of the present invention inherently possessed by the non-human mammals.

The mutant DNA of the present invention includes mutants resulting from variation (e.g., mutation, etc.) in the base sequence of the original DNA of the present invention, specifically DNAs resulting from base addition, deletion, substitution with other bases, etc. and further including abnormal DNA.

The abnormal DNA is intended to mean DNA that expresses the abnormal protein of the present invention and exemplified by DNA that expresses a polypeptide for suppressing the functions of the normal protein of the present invention.

The exogenous DNA of the present invention may be any one of those derived from a mammal of the same species as, or a different species from, the mammal as the target animal. In transfecting the DNA of the present invention, it is generally advantageous to use the DNA as a DNA construct in which the DNA is ligated downstream a promoter capable of expressing the DNA in the target animal. For example, in the case of transfecting the human DNA of the present invention, a DNA transgenic mammal that expresses the DNA of the present invention to a high level, can be prepared by microinjecting a DNA construct (e.g., vector, etc.) ligated with the human DNA of the present invention into a fertilized egg of the target non-human mammal downstream various promoters which are capable of expressing the DNA derived from various mammals (e.g., rabbits, dogs, cats, guinea pigs, hamsters, rats, mice, etc.) bearing the DNA of the present invention highly homologous to the human DNA.

As expression vectors for the polypeptide of the present invention, there are Escherichia coli-derived plasmids, Bacillus subtilis-derived plasmids, yeast-derived plasmids, bacteriophages such as λ phage, retroviruses such as Moloney leukemia virus, etc., and animal viruses such as vaccinia virus, baculovirus, etc. Of these vectors, Escherichia coli-derived plasmids, Bacillus subtilis-derived plasmids, or yeast-derived plasmids, etc. are preferably used.

Examples of,these promoters for regulating the DNA expression include (1) promoters for DNA derived from viruses (e.g., simian virus, cytomegalovirus, Moloney leukemia virus, JC virus, breast cancer virus, poliovirus, etc.), and (2) promoters derived from various mammals (human, rabbits, dogs, cats, guinea pigs, hamsters, rats, mice, etc.), for example, promoters of albumin, insulin II, uroplakin II, elastase, erythropoietin, endothelin, muscular creatine kinase, glial fibrillary acidic protein, glutathione S-transferase, platelet-derived growth factor β, keratins K1, K10 and K14, collagen types I and II, cyclic AMP-dependent protein kinase βI subunit, dystrophin, tartarate-resistant alkaline phosphatase, atrial natriuretic factor, endothelial receptor tyrosine kinase (generally abbreviated as Tie2), sodium-potassium adenosine triphosphorylase (Na,K-ATPase), neurofilament light chain, metallothioneins I and IIA, metalloproteinase I tissue inhibitor, MHC class I antigen (H-2L), H-ras, renin, dopamine β-hydroxylase, thyroid peroxidase (TPO), polypeptide chain elongation factor 1a (EF-1a), β actin, a and β myosin heavy chains, myosin light chains 1 and 2, myelin base protein, thyroglobulins, Thy-1, immunoglobulins, H-chain variable region (VNP), serum amyloid component P, myoglobin, troponin C, smooth muscle, a actin, preproencephalin A, vasopressin, etc. Among them, cytomegalovirus promoters, human polypeptide elongation factor 1a (EF-1a) promoters, human and chicken β actin promoters etc., which protein can highly express in the whole body are preferred.

It is preferred that the vectors described above have a sequence for terminating the transcription of the desired messenger RNA in the DNA transgenic animal (generally termed terminator); for example, a sequence of each DNA derived from viruses and various mammals. SV40 terminator of the simian virus and the like are preferably used.

In addition, for the purpose of increasing the expression of the desired exogenous DNA to a higher level, the splicing signal and enhancer region of each DNA, a portion of the intron of an eukaryotic DNA may also be ligated at the 5′ upstream of the promoter region, or between the promoter region and the translational region, or at the 3′ downstream of the translational region, depending upon purposes.

The translational region for the normal protein of the present invention can be obtained using as a starting material the entire genomic DNA or its portion of liver, kidney, thyroid cell or fibroblast origin from human or various mammals (e.g., rabbits, dogs, cats, guinea pigs, hamsters, rats, mice, etc.) or of various commercially available genomic DNA libraries, or using complementary DNA prepared by a publicly known method from RNA of liver, kidney, thyroid cell or fibroblast origin as a starting material. Alternatively, the translational region for a normal polypeptide translational region obtained by the cell or tissue described above can be made variant by point mutagenesis.

The translational region can be prepared by a conventional DNA engineering technique in which the DNA is ligated downstream the aforesaid promoter and if desired, upstream the translation termination site, as a DNA construct capable of being expressed in the transgenic animal.

The exogenous DNA of the present invention is transfected at the fertilized egg cell stage in a manner such that the DNA is certainly present in all the germinal cells and somatic cells of the target mammal. The fact that the exogenous DNA of the present invention is present in the germinal cells of the animal prepared by DNA transfection means that all offspring of the prepared animal will maintain the exogenous DNA of the present invention in all of the germinal cells and somatic cells thereof. The offspring of the animal that inherits the exogenous DNA of the present invention also have the exogenous DNA in all of the germinal cells and somatic cells thereof.

The non-human mammal in which the normal exogenous DNA of the present invention has been transfected can be passaged as the DNA-bearing animal under ordinary rearing environment, by confirming that the exogenous DNA is stably retained by mating.

By the transfection of the exogenous DNA of the present invention at the fertilized egg cell stage, the DNA is retained to be excess in all of the germinal and somatic cells. The fact that the exogenous DNA of the present invention is excessively present in the germinal cells of the prepared animal after transfection means that the DNA of the present invention is excessively present in all of the germinal cells and somatic cells thereof. The offspring of the animal that inherits the exogenous DNA of the present invention have excessively the DNA of the present invention in all of the germinal cells and somatic cells thereof.

By obtaining a homozygotic animal having the transfected DNA in both of homologous chromosomes and mating a male and female of the animal, all offspring can be passaged to retain the DNA.

In a non-human mammal bearing the normal DNA of the present invention, the normal DNA of the present invention has expressed to a high level, and may eventually develop the function inactive type inadaptability of the protein of the present invention by accelerating the functions of endogenous normal DNA. Therefore, the animal can be utilized as a pathologic model animal for such a disease. Specifically, using the normal DNA transgenic animal of the present invention, it is possible to elucidate the mechanism of the function inactive type inadaptability of the protein of the present invention and the pathological mechanism of the disease associated with the protein of the present invention and to determine how to treat the disease.

Further, since a mammal transfected the exogenous normal DNA of the present invention exhibits an increasing symptom of the protein of the present invention librated, the animal is usable for screening of treatment agent for the disease associated with the protein of the present invention.

On the other hand, non-human mammal having the exogenous abnormal DNA of the present invention can be passaged under normal breeding conditions as the DNA-bearing animal by confirming the stable retaining of the exogenous DNA via crossing. Further, the exogenous DNA to be subjected can be utilized as a starting material by inserting the DNA into the plasmid described above. The DNA construct with promoter can be prepared with conventional DNA engineering techniques. The transfection of the abnormal DNA of the present invention at the fertilized egg cell stage is preserved to be present in all of the germinal and somatic cells of the mammals to be subjected. The fact that the abnormal DNA of the present invention is present in the germinal cells of the animal after DNA transfection means that all of the offspring of the prepared animal have the abnormal DNA of the present invention in all of the germinal and somatic cells. Such an offspring passaged the exogenous DNA of the present invention contains the abnormal DNA of the present invention in all of the germinal and somatic cells. A homozygous animal having the introduced DNA on both of homologous chromosomes can be acquired and then by mating these male and female animals, all the offspring can be bled to have the DNA.

Since non-human mammal having the abnormal DNA of the present invention may express the abnormal DNA of the present invention at a high level, the animal may be the function inactivation type inadaptability of the protein of the present invention by inhibiting the function of the endogenous normal DNA and can be utilized as its disease model animal. For example, using the abnormal DNA-transferred animal of the present invention, it is possible to elucidate the mechanism of inadaptability of the protein of the present invention and to perform to study a method for treatment of this disease.

More specifically, the transgenic animal of the present invention expressing the abnormal DNA of the present invention to a high level is also expected to serve as an experimental model for the elucidation of the mechanism of the functional inhibition (dominant negative effect) of normal protein by the abnormal protein of the present invention in the function inactive type inadaptability of the protein of the present invention.

A mammal bearing the abnormal exogenous DNA of the present invention is also expected to serve for screening a candidate drug for the treatment of the function inactive type inadaptability of the protein of the present invention, since the protein of the present invention is increased in such an animal in its free form.

Other potential applications of two kinds of the transgenic animals described above include:
(i) Use as a cell source for tissue culture;
(ii) Elucidation of the relation to a protein that is specifically expressed or activated by the protein of the present invention, by direct analysis of DNA or RNA in tissue of the DNA transgenic animal of the present invention or by analysis of the polypeptide tissue expressed by the DNA;
(iii) Research in the function of cells derived from tissues that are cultured usually only with difficulty, using cells of tissue bearing the DNA cultured by a standard tissue culture technique;
(iv) Screening for a medicine that enhances the functions of cells using the cells described in (iii) above; and
(v) Isolation and purification of the variant protein of the present invention and preparation of an antibody thereto.

Furthermore, clinical conditions of a disease associated wit the protein of the present invention, including the function inactive type inadaptability of the protein of the present invention can be determined using the DNA transgenic animal of the present invention. In addition, pathological findings on each organ in a disease model associated with the protein of the present invention can be obtained in more detail, leading to the development of a new method for treatment as well as the research and therapy of any secondary diseases associated with the disease.

It is also possible to obtain a free DNA-transfected cell by withdrawing each organ from the DNA transgenic animal of the present invention, mincing the organ and degrading with a proteinase such as trypsin, etc., followed by establishing the line of culturing or cultured cells. Furthermore, the DNA transgenic animal of the present invention can serve as identification of cells capable of producing the protein of the present invention, and as studies on association with apoptosis, differentiation or propagation or on the mechanism of signal transduction in these properties to inspect any abnormality therein. Thus the DNA transgenic animal of the present invention can provide an effective research material for the protein of the present invention and for elucidating the function and effect thereof.

To develop a therapeutic drug for the treatment of diseases associated with the protein of the present invention, including the function inactive type inadaptability of the protein of the present invention, using the DNA transgenic animal of the present invention, an effective and rapid method for screening can be provided by using the method for inspection and the method for quantification, etc. described above. It is also possible to investigate and develop a method for DNA therapy for the treatment of diseases associated with the protein of the present invention, using the DNA transgenic animal of the present invention or a vector capable of expressing the exogenous DNA of the present invention.

### [8] Knockout Animals

The present invention provides a non-human mammalian embryonic stem cell, wherein the DNA of the present invention is inactivated, and a non-human mammal deficient in expressing the DNA of the present invention.

That is, the present invention provides:
(1) A non-human embryonic stem cell in which the DNA of the present invention is inactivated;
(2) The embryonic stem cell according to (1), wherein the DNA is inactivated by introducing a reporter gene (e.g., β-galactosidase gene derived from Escherichia coli);
(3) The embryonic stem cell according to (1), which is resistant to neomycin;
(4) The embryonic stem cell according to (1), wherein the non-human mammal is a rodent;
(5) The embryonic stem cell according to (4), wherein the rodent is mouse;
(6) A non-human mammal deficient in expressing the DNA of the present invention, wherein the DNA of the present invention is inactivated;
(7) The non-human mammal according to (6), wherein the DNA is inactivated by inserting a reporter gene (e.g., β-galactosidase derived from Escherichia coli) therein and the reporter gene is capable of being expressed under control of a promoter for the DNA of the present invention;
(8) The non-human mammal according to (6), which is a rodent;
(9) The non-human mammal according to (8), wherein the rodent is mouse; and
(10) A method for screening a compound or its salt that promotes or inhibits the promoter activity for the DNA of the present invention, which comprises administering a test compound to the mammal of (7) and detecting expression of the reporter gene.

The non-human mammal embryonic stem cell in which the DNA of the present invention is inactivated refers to a non-human mammal embryonic stem cell that suppresses the ability of the non-human mammal to express the DNA by artificially mutating the DNA of the present invention, or the DNA has no substantial ability to express the polypeptide of the present invention (hereinafter sometimes referred to as the knockout DNA of the present invention) by substantially inactivating the activities of the polypeptide of the present invention encoded by the DNA (hereinafter merely referred to as ES cell).

As the non-human mammal, the same examples as described above apply.

Methods for artificially mutating the DNA of the present invention include, for example, deletion of a part or all of the DNA sequence and insertion of or substitution with other DNA, by genetic engineering. By these mutations, the knockout DNA of the present invention may be prepared, for example, by shifting the reading frame of a codon or by disrupting the function of a promoter or exon.

Specifically, the non-human mammal embryonic stem cell in which the DNA of the present invention is inactivated (hereinafter merely referred to as the ES cell with the DNA of the present invention inactivated or the knockout ES cell of the present invention) can be obtained by, for example, isolating the DNA of the present invention that the desired non-human mammal possesses, inserting a DNA fragment having a DNA sequence constructed by inserting a drug resistant gene such as a neomycin resistant gene or a hygromycin resistant gene, or a reporter gene such as lacZ (β-galactosidase gene) or cat (chloramphenicol acetyltransferase gene), etc. into its exon site thereby to disable the functions of exon, or integrating to a chromosome of the subject animal by, e.g., homologous recombination, a DNA sequence which terminates gene transcription (e.g., polyA additional signal, etc.) in the intron between exons to, thus inhibit the synthesis of complete messenger RNA and eventually destroy the gene (hereinafter simply referred to as targeting vector). The thus-obtained ES cells to the Southern hybridization analysis with a DNA sequence on or near the DNA of the present invention as a probe, or to PCR analysis with a DNA sequence on the targeting vector and another DNA sequence near the DNA of the present invention which is not included in the targeting vector as primers, to select the knockout ES cell of the present invention.

The parent ES cells to inactivate the DNA of the present invention by homologous recombination, etc. may be of a strain already established as described above, or may be originally established in accordance with a modification of the publicly known method by Evans and Kaufman supra. For example, in the case of mouse ES cells, currently it is common practice to use ES cells of the 129 strain. However, since their immunological background is obscure, the C57B116 mouse or the BDF₁ mouse (F₁ hybrid between C57BL/6 and DBA/2), wherein the low ovum availability per C57BL/6 in the C57BL/6 mouse has been improved by crossing with DBA/2, may be preferably used, instead of obtaining a pure line of ES cells with the clear immunological genetic background and for other purposes. The BDF, mouse is advantageous in that, when a pathologic model mouse is generated using ES cells obtained therefrom, the genetic background can be changed to that of the C57BL/6 mouse by back-crossing with the C57B116 mouse, since its background is of the C57BL/6 mouse, as well as being advantageous in that ovum availability per animal is high and ova are robust.

In establishing ES cells, blastocytes at 3.5 days after fertilization are commonly used. Except for this fact, by collecting embryos at the 8-cell stage and culturing those until the blastocyte stage, a large number of early stage embryos may efficiently be obtained.

Although the ES cells used may be of either sex, male ES cells are generally more convenient for generation of a germ cell line chimera and are therefore preferred. It is desirable to identify sexes as soon as possible also in order to save painstaking culture time.

Methods for sex identification of the ES cell include the method in which a gene in the sex-determining region on the Y-chromosome is amplified by the PCR process and detected. When this method is used, one colony of ES cells (about 50 cells) is sufficient for sex-determination analysis, which karyotype analysis, for example G-banding method, requires about 10⁶ cells; therefore, the first selection of ES cells at the early stage of culture can be based on sex identification, and male cells can be selected early, which saves a significant amount of time at the early stage of culture.

Second selection can be achieved by, for example, number of chromosome confirmation by the G-banding method. It is usually desirable that the chromosome number of the obtained ES cells be 100% of the normal number. However, when it is difficult to obtain the cells having the normal number of chromosomes due to physical operation etc. in cell establishment, it is desirable that the ES cell be again cloned to a normal cell (e.g., in mouse cells having the number of chromosomes being 2n=40) after the gene of the ES cells is rendered knockout.

Although the embryonic stem cell line thus obtained shows a very high growth potential, it must be subcultured with great care, since it tends to lose its ontogenic capability. For example, the embryonic stem cell line is cultured at about 37° C. in a carbon dioxide incubator (preferably about 5% carbon dioxide and about 95% air, or about 5% oxygen, about 5% carbon dioxide and 90% air) in the presence of LIF (1-10000 U/ml) on appropriate feeder cells such as STO fibroblasts, treated with a trypsin/EDTA solution (normally about 0.001 % to about 0.5% trypsin/about 0.1 to about 5 mM EDTA, preferably about 0.1% trypsin/1 mM EDTA) at the time of passage to obtain separate single cells, which are then seeded on freshly prepared feeder cells. This passage is normally conducted every 1 to 3 days; it is desirable that cells be observed at passage and cells found to be morphologically abnormal in culture, if any, be abandoned.

By allowing ES cells to reach a high density in mono-layers or to form cell aggregates in suspension under appropriate conditions, it is possible to spontaneously differentiate them to various cell types, for example, pariental muscle, visceral muscles, cardiac muscle or the like (M. J. Evans and M. H. Kaufman, Nature, 292, 154, 1981; G. R. Martin, Proc. Natl. Acad. Sci. U.S.A., 78, 7634, 1981; T. C. Doetschman et al., Journal of Embryology Experimental Morphology, 87, 27, 1985). The cells deficient in expression of the DNA of the present invention, which are obtainable from the differentiated ES cells of the present invention, are useful for studying the functions of the polypeptide of the present invention cytologically or molecular biologically.

The non-human mammal deficient in expression of the DNA of the present invention can be identified from a normal animal by measuring the amount of mRNA in the subject animal by a publicly known method, and indirectly comparing the degrees of expression.

As the non-human mammal, the same examples supra can be applied.

With respect to the non-human mammal deficient in expression of the DNA of the present invention, the DNA of the present invention can be made knockout by transfecting a targeting vector, prepared as described above, to mouse embryonic stem cells or mouse oocytes, and conducting homologous recombination in which a targeting vector DNA sequence, wherein the DNA of the present invention is inactivated by the transfection, is replaced with the DNA of the present invention on a chromosome of a mouse embryonic stem cell or mouse oocyte.

The knockout cells with the DNA of the present invention disrupted can be identified by Southern hybridization analysis with a DNA sequence on or near the DNA of the present invention as a probe, or by PCR analysis using a DNA sequence on the targeting vector and another DNA sequence derived from mouse, which is not included in the targeting vector, as primers. When non-human mammalian embryonic stem cells are used, a cell line wherein the DNA of the present invention is inactivated by homologous recombination is cloned; the resulting cloned cell line is injected to, e.g., a non-human mammalian embryo or blastocyte, at an appropriate stage such as the 8-cell stage. The resulting chimeric embryos are transplanted to the uterus of the pseudopregnant non-human mammal. The prepared animal is a chimeric animal composed of both cells having the normal locus of the DNA of the present invention and those having an artificially mutated locus of the DNA of the present invention.

When some germ cells of the chimeric animal have a mutated locus of the DNA of the present invention, an individual, which entire tissue is composed of cells having a mutated locus of the DNA of the present invention can be selected from a series of offspring obtained by crossing between such a chimeric animal and a normal animal, e.g., by coat color identification, etc. The individuals thus obtained are normally deficient in heterozygous expression of the receptor protein of the present invention. The individuals deficient in homozygous expression of the receptor protein of the present invention can be obtained from offspring of the intercross between the heterozygotes.

When an oocyte is used, a DNA solution may be injected, e.g., to the nucleus of the oocyte by microinjection thereby to obtain a transgenic non-human mammal having a targeting vector introduced in a chromosome thereof. From such transgenic non-human mammals, those having a mutation at the locus of the DNA of the present invention can be obtained by selection based on homologous recombination.

As described above, individuals in which the DNA of the present invention is rendered knockout permit passage rearing under ordinary rearing conditions, after the individuals obtained by their crossing have proven to have been knockout.

Furthermore, the genital system may be obtained and maintained by conventional methods. That is, by crossing male and female animals each having the inactivated DNA, homozygoous animals having the inactivated DNA in both loci can be obtained. The homozygotes thus obtained may be reared so that one normal animal and two or more homozygotes are produced from a mother animal to efficiently obtain such homozygotes. By crossing male and female heterozygotes, homozygotes and heterozygotes having the inactivated DNA are proliferated and passaged.

The non-human mammal embryonic stem cell in which the DNA of the present invention is inactivated is very useful for preparing a non-human mammal deficient in expression of the DNA of the present invention.

Since the non-human mammal in which the DNA of the present invention is inactivated lacks various biological activities derived from the protein of the present invention, such an animal can be a disease model suspected of inactivated biological activities of the protein of the present invention and thus, offers an effective study to investigate causes for and therapy for these diseases.

### [8a] A Method for Screening of Compounds having Therapeutic and/or Prophylactic Effects for Diseases Caused by Deficiency, Damages, etc. of the DNA of the Present Invention

The non-human mammal deficient in expression of the DNA of the present invention can be employed for the screening of compounds having therapeutic and/or prophylactic effects for diseases caused by deficiency, damages, etc. of the DNA of the present invention.

That is, the present invention provides a method for screening of a compound having therapeutic and/or prophylactic effects for diseases caused by deficiency, damages, etc. of the DNA of the present invention, which comprises administering a test compound to the non-human mammal deficient in expression of the DNA of the present invention and observing and measuring a change occurred in the animal.

As the non-human mammal deficient in expression of the DNA of the present invention, which can be employed for the screening method, the same examples as given hereinabove apply.

Examples of the test compounds include peptides, proteins, non-peptide compounds, synthetic compounds, fermentation products, cell extracts, plant extracts, animal tissue extracts, blood plasma and the like. These compounds may be novel compounds or publicly known compounds.

Specifically, the non-human mammal deficient in the expression of the DNA of the present invention is treated with a test compound, and by comparison with an intact animal for control, a change in each organ, tissue, disease conditions, etc. of the animal is used as an index to assess the therapeutic and/or prophylactic effects of the test compound.

For treating an animal to be tested with a test compound, for example, oral administration, intravenous injection, etc. are applied and the treatment is appropriately selected depending upon conditions of the test animal, properties of the test compound, etc. Furthermore, an amount of a test compound administered can be selected depending on administration route, nature of the test compound, and the like.

For example, in the case of screening a compound having a therapeutic and/or prophylactic effect for irritable bowel syndrome, a test compound may be administered to the non-human mammal deficient in the expression of the DNA of the present invention. Then changes in amount of bowel movement depending on stress caused by restriction, is measured with time.

The compound obtained using the above screening method is a compound selected from the test compounds described above and exhibits a therapeutic and/or prophylactic effect for the diseases caused by deficiencies, damages, etc. of the protein of the present invention. Therefore, the compound can be employed as a safe and low toxic pharmaceutical such as the prophylactic and/or therapeutic agent for these diseases. Furthermore, compounds derived from such a compound obtained by the above screening can be likewise employed.

The compound obtained by the screening method may be used in the form of salts with physiologically acceptable acids (e.g., inorganic acids or organic acids) or bases (e.g., alkali metal salts), preferably in the form of physiologically acceptable acid addition salts. Examples of such salts are salts with inorganic acids (e.g., hydrochloric acid, phosphoric acid, hydrobromic acid, sulfuric acid), salts with organic acids (e.g., acetic acid, formic acid, propionic acid, fumaric acid, maleic acid, succinic acid, tartaric acid, citric acid, malic acid, oxalic acid, benzoic acid, methanesulfonic acid, benzenesulfonic acid) and the like.

A pharmaceutical containing the compound obtained by the above screening method or salts thereof may be manufactured in a manner similar to the method for preparing the pharmaceutical comprising the protein of the present invention described hereinabove.

Since the preparation thus obtained is safe and low toxic, it can be administered to human or other mammals (e.g., rat, mouse, guinea pig, rabbit, sheep, swine, bovine, horse, cat, dog, monkey, etc.).

Although the amount of the compound or its salt to be administered varies depending upon particular disease, subject to be administered, route of administration, etc., but when the compound is orally administered, the compound is generally administered to an adult patient with irritable bowel syndrome (as 60 kg body weight) in a dose of about 0.1 mg/day to about 100 mg/day, preferably about 1.0 mg/day to about 50 mg/day, more preferably about 1.0 mg to about 20 mg. For parenteral administration, a single dose of the compound varies depending upon subject to be administered, target disease, etc., but when the compound is administered to an adult patient with irritable bowel syndrome (as 60 kg body weight) in the form of an injectable preparation, it is generally advantageous to administer the compound intravenously in a dose of about 0.01 mg/day to about 30 mg/day, preferably about 0.1 mg/day to about 20 mg/day, more preferably about 0.1 mg/day to about 10 mg/day. As for other animals, the compound can be administered in the above amount with converting it into that for the body weight of 60 kg.

### [8b] A Method for Screening a Compound that Promotes or Inhibits the Activities of a Promoter to the DNA of the Present Invention

The present invention provides a method for screening a compound or its salt that promotes or inhibits the activities of a promoter to the DNA of the present invention, which comprises administering a test compound to a non-human mammal deficient in expression of the DNA of the present invention and detecting expression of the reporter gene.

In the screening method described above, as the non-human mammal deficient in expression of the DNA of the present invention, an animal in which the DNA of the present invention is inactivated by introducing a reporter gene and the reporter gene can be expressed under control of a promoter to the DNA of the present invention is used from the aforementioned non-human mammal deficient in expression of the DNA of the present invention.

The same examples of the test compound apply to specific compounds used for the screening.

As the reporter gene, the same specific examples described above apply, and β-galactosidase (lacZ), soluble alkaline phosphatase gene, luciferase gene and the like are preferably employed.

Since a reporter gene is present under control of a promoter to the DNA of the present invention in the non-human mammal deficient in expression of the DNA of the present invention wherein the DNA of the present invention is substituted with the reporter gene, the activity of the promoter can be detected by tracing the expression of a substance encoded by the reporter gene.

When a part of the DNA region encoding the protein of the present invention is substituted with, e.g., β-galactosidase gene (lacZ) derived from Escherichia coli, β-galactosidase is expressed in a tissue where the protein of the present invention should originally be expressed, instead of the protein of the present invention. Thus, the state of expression of the protein of the present invention can be readily observed in vivo of an animal by staining with a reagent such as 5-bromo-4-chloro-3-indolyl-β-galactopyranoside (X-gal), which is substrate for β-galactosidase. Specifically, a mouse deficient in the protein of the present invention, or its tissue section is fixed with glutaraldehyde, etc. After washing with phosphate buffered saline (PBS), the system is reacted with a staining solution containing X-gal at room temperature or about 37° C. for approximately 30 minutes to an hour. After that, by washing the tissue preparation with 1 mM EDTA/PBS solution, the β-galactosidase reaction is terminated, and the color formed is observed. Alternatively, mRNA encoding lacZ may be detected in a conventional manner.

The compound or salts thereof obtained using the above screening method, are compounds that are selected from the test compounds described above and that promote or inhibit the promoter activity to the DNA of the present invention.

The compound obtained by the screening method above may be used in the form of salts with physiologically acceptable acids (e.g., inorganic acids or organic acids) or bases (e.g., alkali metal salts), preferably in the form of physiologically acceptable acid addition salts. Examples of such salts are salts with inorganic acids (e.g., hydrochloric acid, phosphoric acid, hydrobromic acid, sulfuric acid), salts with organic acids (e.g., acetic acid, formic acid, propionic acid, fumaric acid, maleic acid, succinic acid, tartaric acid, citric acid, malic acid, oxalic acid, benzoic acid, methanesulfonic acid, benzenesulfonic acid) and the like.

Since the compounds or salts thereof that enhance the promoter activity to the DNA of the present invention can promote the expression of the protein of the present invention, or can promote the functions of the protein, they are useful as pharmaceuticals such as the prophylactic and/or therapeutic agent for alimentary diseases (e.g., irritable bowel syndrome, chronic ulcerative colitis, Crohn's disease, ischemic colitis, gastritis, peptic ulcer, proctitis, diarrhea, reflux esophagitis, duodenitis, etc.), reproductive system diseases (e.g., prostate hyperplasia, prostatitis, testis neurosis, ovarian cystoma, etc.), respiratory diseases (e.g., chronic obstructive pulmonary disease, asthma, etc.), central nervous system diseases (e.g., Alzheimer's disease, parkinsonian syndrome, schizophrenia, cerebrovascular dementia, cerebral ischemia, convulsions, etc.), diabetes mellitus, hypertension, circulatory diseases (e.g., heart failure, arrhythmia, long QT syndrome, arteriosclerosis, angina, etc.), pancreatic diseases (e.g., pancreatitis, hypopancreatism such as cystic fibrosis pancreas, etc.), liver diseases (e.g., cirrhosis hepatic, etc.), kidney diseases (e.g., kidney failure, uremia, etc.), cancer (e.g., lung cancer, kidney cancer, liver cancer, non-small cell lung cancer, ovarian cancer, prostate cancer, stomach cancer, bladder cancer, breast cancer, cancer of uterine cervix, colon cancer, rectum cancer, pancreatic cancer, tymoma, testicular tumor, esophageal cancer, myosarcoma, etc.), or the like.

Further, the compounds or salts thereof that inhibit the promoter activity to the DNA of the present invention can inhibit the expression of the protein of the present invention, or can inhibit the functions of the protein, they are useful as a prophylactic and/or therapeutic agent for alimentary diseases (e.g., irritable bowel syndrome, chronic ulcerative colitis, Crohn's disease, ischemic colitis, gastritis, peptic ulcer, proctitis, diarrhea, reflux esophagitis, duodenitis, etc.), reproductive system diseases (e.g., prostate hyperplasia, prostatitis, testis neurosis, ovarian cystoma, etc.), respiratory diseases (e.g., chronic obstructive pulmonary disease, asthma, etc.), central nervous system diseases (e.g., Alzheimer's disease, parkinsonian syndrome, schizophrenia, cerebrovascular dementia, cerebral ischemia, convulsions, etc.), diabetes mellitus, hypertension, circulatory diseases (e.g., heart failure, arrhythmia, long QT syndrome, arteriosclerosis, angina, etc.), pancreatic diseases (e.g., pancreatitis, hypopancreatism such as cystic fibrosis pancreas, etc.), liver diseases (e.g., cirrhosis hepatic, etc.), kidney diseases (e.g., kidney failure, uremia, etc.), cancer (e.g., lung cancer, kidney cancer, liver cancer, non-small cell lung cancer, ovarian cancer, prostate cancer, stomach cancer, bladder cancer, breast cancer, cancer of uterine cervix, colon cancer, rectum cancer, pancreatic cancer, tymoma, testicular tumor, esophageal cancer, myosarcoma, etc.), or the like.

In addition, compound derived from the compounds obtained by the screening above may be likewise employed.

A pharmaceutical containing the compounds or salts thereof obtained by the screening method may be manufactured in a manner similar to the method for preparing the pharmaceutical containing the protein of the present invention described above.

Since the pharmaceutical preparation thus obtained is safe and low toxic, it can be administered to human or other mammals (e.g., rat, mouse, guinea pig, rabbit, sheep, swine, bovine, horse, cat, dog, monkey, etc.).

The dose of the compound or salts thereof varies depending on target disease, subject to be administered, route for administration, etc.; for example, when the compound that enhances the promoter activity to the DNA of the present invention is orally administered, the dose is normally about 0.1 to about 100 mg, preferably about 1.0 to about 50 mg, more preferably about 1.0 to about 20 mg per day for adult patient with irritable bowel syndrome (as 60 kg body weight). In parenteral administration, a single dose of the compound varies depending on subject to be administered, target disease, etc. but when the compound that enhances the promoter activity to the DNA of the present invention is administered in the form of injectable preparation, it is advantageous to administer the compound intravenously at a single dose of about 0.01 to about 30 mg/day, preferably about 0.1 to about 20 mg/day, more preferably about 0.1 to about 10 mg/day for adult patient with irritable bowel syndrome (as 60 kg body weight). For other animal species, the corresponding dose as converted per 60 kg weight can be administered.

On the other hand, when the compound that inhibits the promoter activity to the DNA of the present invention is orally administered, the dose is normally about 0.1 to about 100 mg, preferably about 1.0 to about 50 mg, more preferably about 1.0 to about 20 mg per day for adult patient with irritable bowel syndrome (as 60 kg body weight). In parenteral administration, a single dose of the compound varies depending on subject to be administered, target disease, etc. but when the compound that inhibits the promoter activity to the DNA of the present invention is administered in the form of injectable preparation, it is advantageous to administer the compound intravenously at a single dose of about 0.01 to about 30 mg/day, preferably about 0.1 to about 20 mg/day, more preferably about 0.1 to about 10 mg/day for adult patient with irritable bowel syndrome (as 60 kg body weight). For other animal species, the corresponding dose as converted per 60 kg weight can be administered.

As stated above, the non-human mammal deficient in expression of the DNA of the present invention is extremely useful for screening the compound or its salt that promotes or inhibits the activity of a promoter to the DNA of the present invention and can greatly contribute to the elucidation of causes for various diseases suspected of deficiency in expression of the DNA of the present invention and for the development of medicine for prevention and/or treatment of these diseases.

Furthermore, a so-called transgenic animal (gene introduced animal) can be prepared by using DNA containing a promoter region of the protein of the present invention, ligating genes encoding various proteins downstream and injecting the same into oocyte of an animal. It is then possible to synthesize the protein therein specifically and study its activity in vivo. When an appropriate reporter gene is ligated to the promoter site above and a cell line that express the gene is established, the resulting system can be utilized as the survey system for a low molecular compound having the action of specifically promoting or inhibiting the in vivo productivity of the protein per se of the present invention.

In the specification and drawings, the codes of bases and amino acids are denoted in accordance with the IUPAC-IUB Commission on Biochemical Nomenclature or by the common codes in the art, examples of which are shown below. For amino acids that may have the optical isomer, L form is presented unless otherwise indicated.

| | |
|---|---|
| DNA | deoxyribonucleic acid |
| cDNA | complementary deoxyribonucleic acid |
| A | adenine |
| T | thymine |
| G | guanine |
| C | cytosine |
| RNA | ribonucleic acid |
| mRNA | messenger ribonucleic acid |
| dATP | deoxyadenosine triphosphate |
| dTTP | deoxythymidine triphosphate |
| dGTP | deoxyguanosine triphosphate |
| dCTP | deoxycytidine triphosphate |
| ATP | adenosine triphosphate |
| EDTA | ethylenediaminetetraacetic acid |
| SDS | sodium dodecyl sulfate |
| Gly | glycine |
| Ala | alanine |
| Val | valine |
| Leu | leucine |
| Ile | isoleucine |
| Ser | serine |
| Thr | threonine |
| Cys | cysteine |
| Met | methionine |
| Glu | glutamic acid |
| Asp | aspartic acid |
| Lys | lysine |
| Arg | arginine |
| His | histidine |
| Phe | phenylalanine |
| Tyr | tyrosine |
| Trp | tryptophan |
| Pro | proline |
| Asn | asparagine |
| Gln | glutamine |
| pGlu | pyroglutamic acid |

The substituents, protective groups and reagents, which are frequently used throughout the specification, are shown by the following abbreviations.

| | |
|---|---|
| Me | methyl group |
| Et | ethyl group |
| Bu | butyl group |
| Ph | phenyl group |
| TC | thiazolidine-4(R)-carboxamide group |
| Tos | p-toluenesulfonyl |
| CHO | formyl |
| Bzl | benzyl |
| Cl₂-Bzl | 2,6-dichlorobenzyl |
| Bom | benzyloxymethyl |
| Z | benzyloxycarbonyl |
| Cl-Z | 2-chlorobenzyloxycarbonyl |
| Br-Z | 2-bromobenzyloxycarbonyl |
| Boc | t-butoxycarbonyl |
| DNP | dinitrophenol |
| Trt | trityl |
| Bum | t-butoxymethyl |
| Fmoc | N-9-fluorenylmethoxycarbonyl |
| HOBt | 1-hydroxybenztriazole |
| HOOBt | 3,4-dihydro-3-hydroxy-4-oxo-1,2,3-benzotriazine |
| HONB | 1-hydroxy-5-norbornene-2,3-dicarboximide |
| DCC | N,N′-dicyclohexylcarbodiimide |

The sequence identification numbers in the sequence listing of the specification indicates the following sequence, respectively.

[SEQ ID NO: 1]

This shows the amino acid sequence of human TCH204 variant 1 protein, which was acquired in EXAMPLE 1.

[SEQ ID NO: 2]

This shows the base sequence of DNA encoding human TCH204 variant 1 protein, which possesses the amino acid sequence represented by SEQ ID NO: 1.

[SEQ ID NO: 3]

This shows the base sequence of synthetic DNA primer used in EXAMPLE 1 and EXAMPLE 2.

[SEQ ID NO: 4]

This shows the base sequence of synthetic DNA primer used in EXAMPLE 1 and EXAMPLE 2.

[SEQ ID NO: 5]

This shows the base sequence of synthetic DNA primer used in EXAMPLE 1, EXAMPLE 2 and EXAMPLE 4.

[SEQ ID NO: 6]

This shows the base sequence of synthetic DNA primer used in EXAMPLE 1, EXAMPLE 2 and EXAMPLE 4.

[SEQ ID NO: 7]

This shows the base sequence of DNA encoding human TCH204 variant 1, which was acquired in EXAMPLE 1 and inserted into plasmid.

[SEQ ID NO: 8]

This shows the amino acid sequence of human TCH204 variant 2 protein, which was acquired in EXAMPLE 2.

[SEQ ID NO: 9]

This shows the base sequence of DNA encoding human TCH204 variant 2 protein, which possesses the amino acid sequence represented by SEQ ID NO: 8.

[SEQ ID NO: 10]

This shows the base sequence of DNA encoding human TCH204 variant 2, which was acquired in EXAMPLE 2 and inserted into plasmid.

[SEQ ID NO: 11]

This shows the amino acid sequence of human TCH204 variant 3 protein, which was acquired in EXAMPLE 3.

[SEQ ID NO: 12]

This shows the base sequence of DNA encoding human TCH204 variant 3 protein, which possesses the amino acid sequence represented by SEQ ID NO: 11.

[SEQ ID NO: 13]

This shows the base sequence of DNA encoding human TCH204 variant 3, which was acquired in EXAMPLE 3 and inserted into plasmid.

[SEQ ID NO: 14]

This shows the base sequence of synthetic DNA primer used in EXAMPLE 4.

[SEQ ID NO: 15]

This shows the amino acid sequence of human TCH204 variant 4 protein, which was acquired in EXAMPLE 4.

[SEQ ID NO: 16]

This shows the base sequence of DNA encoding human TCH204 variant 4 protein, which possesses the amino acid sequence represented by SEQ ID NO: 15.

[SEQ ID NO: 17]

This shows the base sequence of DNA encoding human TCH204 variant 4, which was acquired in EXAMPLE 4 and inserted into plasmid.

[SEQ ID NO: 18]

This shows the amino acid sequence of mouse TCH204 variant 6 protein, which was acquired in EXAMPLE 5.

[SEQ ID NO: 19]

This shows the base sequence of DNA encoding mouse TCH204 variant 6 protein, which possesses the amino acid sequence represented by SEQ ID NO: 18.

[SEQ ID NO: 20]

This shows the base sequence of primer F02 used in EXAMPLE 5.

[SEQ ID NO: 21]

This shows the base sequence of primer R02 used in EXAMPLE 5.

[SEQ ID NO: 22]

This shows the base sequence of DNA encoding mouse TCH204 variant 6, which was acquired in EXAMPLE 5 and inserted into plasmid.

[SEQ ID NO: 23]

This shows the amino acid sequence of mouse TCH204 variant 5 protein, which was acquired in EXAMPLE 6.

[SEQ ID NO: 24]

This shows the base sequence of DNA encoding mouse TCH204 variant 5 protein, which possesses the amino acid sequence represented by SEQ ID NO: 23.

[SEQ ID NO: 25]

This shows the base sequence of DNA encoding mouse TCH204 variant 5, which was acquired in EXAMPLE 6 and inserted into plasmid.

[SEQ ID NO: 26]

This shows the amino acid sequence of mouse TCH204 variant 2 protein, which was acquired in EXAMPLE 7.

[SEQ ID-NO: 27]

This shows the base sequence of DNA encoding mouse TCH204 variant 2 protein, which possesses the amino acid sequence represented by SEQ ID NO: 26.

[SEQ ID NO: 28]

This shows the base sequence of DNA encoding mouse TCH204 variant 2, which was acquired in EXAMPLE 7 and inserted into plasmid.

[SEQ ID NO: 29]

This shows the base sequence of synthetic DNA primer used in EXAMPLE 8.

[SEQ ID NO: 30]

This shows the base sequence of synthetic DNA primer used in EXAMPLE 8.

[SEQ ID NO: 31]

This shows the base sequence of synthetic DNA probe for TaqMan PCR used in EXAMPLE 8.

[SEQ ID NO: 32]

This shows the base sequence of synthetic DNA primer used in EXAMPLE 8.

[SEQ ID NO: 33]

This shows the base sequence of synthetic DNA probe for TaqMan PCR used in EXAMPLE 8.

[SEQ ID NO: 34]

This shows the base sequence of synthetic DNA primer used in EXAMPLE 8.

[SEQ ID NO: 35]

This shows the base sequence of synthetic DNA probe for TaqMan PCR used in EXAMPLE 8.

[SEQ ID NO: 36]

This shows the base sequence of synthetic DNA primer used in EXAMPLE 8.

[SEQ ID NO: 37]

This shows the base sequence of synthetic DNA primer used in EXAMPLE 8.

[SEQ ID NO: 38]

This shows the base sequence of synthetic DNA probe for TaqMan PCR used in EXAMPLE 8.

[SEQ ID NO: 39]

This shows the base sequence of synthetic DNA primer used in EXAMPLE 8.

[SEQ ID NO: 40]

This shows the base sequence of synthetic DNA primer used in EXAMPLE 8.

[SEQ ID NO: 41]

This shows the base sequence of synthetic DNA probe for TaqMan PCR used in EXAMPLE 8.

[SEQ ID NO: 42]

This shows the base sequence of synthetic DNA primer used in EXAMPLE 8.

[SEQ ID NO: 43]

This shows the base sequence of synthetic DNA primer used in EXAMPLE 8.

[SEQ ID NO: 44]

This shows the base sequence of synthetic DNA probe for TaqMan PCR used in EXAMPLE 8.

The transformant Escherichia coli TOPO10/pCRII-TCH204V1 obtained in EXAMPLE 1 described below was as Escherichia coli TOP10/pCRII-TCH204V1 on deposit with International Patent Organisms Depository, National Institute of Advanced Industrial Science and Technology, located at Central 6, 1-1-1 Higashi, Tsukuba, Ibaraki, 305-8566, Japan, as the Accession Number FERM BP-8053 on May 27, 2002 and with Institute for Fermentation (IFO), located at 2-17-85 Juso-honmachi, Yodogawa-ku, Osaka-shi, Osaka, Japan, as the Accession Number IFO 16794 on May 14, 2002.

The transformant Escherichia coli TOPO10/pCRII-TCH204V2 obtained in EXAMPLE 2 described below was as Escherichia coli TOP10/pCRII-TCH204V2 on deposit with International Patent Organisms Depository, National Institute of Advanced Industrial Science and Technology, located at Central 6, 1-1-1 Higashi, Tsukuba, Ibaraki, 305-8566, Japan, as the Accession Number FERM BP-8054 on May 27, 2002 and with Institute for Fermentation (IFO), located at 2-17-85 Juso-honmachi, Yodogawa-ku, Osaka-shi, Osaka, Japan, as the Accession Number IFO 16795 on May 14, 2002. The transformant Escherichia coli TOPO10/pCRII-mTCH204V6 obtained in EXAMPLE 5 described below was as Escherichia coli TOP10/pCRII-mTCH204V6 on deposit with International Patent Organisms Depository, National Institute of Advanced Industrial Science and Technology, located at Central 6, 1-1-1 Higashi, Tsukuba, Ibaraki, 305-8566, Japan, as the Accession Number FERM BP-8119 on Jul. 16, 2002.

The present invention is described in detail below with reference to EXAMPLES, but is not deemed to limit the scope of the present invention thereto. The gene manipulation procedures using Escherichia coli were performed according to the methods described in the Molecular Cloning.

### EXAMPLE 1

Cloning of cDNA Coding for Human TCH204 Variant 1 Gene

Using the following synthetic DNA primers and human lung-derived cDNA as a template, amplification by PCR method was performed. The reaction solution comprised of 2.0 μl of human lung Marathon-Ready cDNA (CLONTECH), 2 μM each of synthetic DNA primers (SEQ ID NO: 3 and SEQ ID NO: 4), 0.2 mM dNTPs, 0.125 μl of Pyrobest DNA Polymerase (Takara Shuzo) and Pyrobest buffer attached to the enzyme to make the total volume 20 μl. The cycle for amplification was carried out using a thermal cycler (PE Biosystems) by heating of 94° C. for 5 minutes, then a cycle set to include 94° C. for 20 seconds followed by 72° C. for 5 minutes, which was repeated 5 times, 94° C. for 20 seconds followed by 70° C. for 5 minutes, which was repeated 5 times, 94° C. for 20 seconds followed by 68° C. for 5 minutes, which was repeated 30 times, and finally, incubation at 72° C. for 5 minutes. Then, the reaction solution comprised of 5 μl of the PCR reaction solution diluted 50-fold with DNase-, RNase-free distilled water, 2 μM each of synthetic DNA primers (SEQ ID NO: 5 and SEQ ID NO: 6), 0.2 mM dNTPs, 0.313 μl of Pyrobest DNA Polymerase (Takara Shuzo) and Pyrobest buffer attached to the enzyme to make the total volume 50 μl. The cycle for amplification was carried out using a thermal cycler (PE Biosystems) by heating of 94° C. for 5 minutes, then a cycle set to include 94° C. for 20 seconds followed by 72° C. for 5 minutes, which was repeated 5 times, 94° C. for 20 seconds followed by 70° C. for 5 minutes, which was repeated 5 times, 94° C. for 20 seconds followed by 68° C. for 5 minutes, which was repeated 30 times, and finally, incubation at 72° C. for 5 minutes. The amplified DNA was isolated by 1.5% agarose gel electrophoresis and about 3.5 kb band was excised by razor blade. The DNA was recovered using the QIAquick Gel Extraction Kit (QIAGEN). The DNA was cloned into pCRII-TOPO vector in accordance with the protocol of TOPO TA Cloning Kit Dual Promoter (Invitrogen). After transformation of Escherichia coli TOP10 competent cell (Invitrogen) by introducing the above-mentioned vector, clones harboring cDNA insert fragment were selected on LB agar medium containing kanamycin and X-gal. All the white-colored clones were isolated with sterilized toothpick, and then the transformants were obtained. Respective clones were cultured in LB medium containing kanamycin for overnight. Subsequently, the plasmid DNA was prepared using QIAprep Miniprep (QIAGEN). The reaction for determination of the base sequence was carried out using BigDye Terminator Cycle Sequencing Ready Reaction Kit (Applied Biosystems). As a result, with the DNA sequencer, ABI PPISM 3100 Analyzer (Applied Biosystems), the base sequence of cDNA fragment inserted was decoded.

The 3517 bases were inserted into the acquired plasmid (SEQ ID NO: 7), and ORF encoding 1118 amino acid residues were present in the cDNA fragment (SEQ ID NO: 1). The protein containing the amino acid sequence represented by SEQ ID NO: 1 was designated human TCH204 variant 1 protein. The transformant having the plasmid containing the above-mentioned cDNA fragment (SEQ ID NO: 7) was designated Escherichia coli TOP10/pCRII-TCH204V1.

Whereas the 2916th base in the sequence represented by SEQ ID NO: 2 was A, the corresponding base of the other human TCH204 variant obtained in EXAMPLES 2, 3 and 4 described later was G. It is considered that the base substitution was occurred by SNP. In addition, the base encodes Gln, the 972nd residue of the amino acid sequence represented by SEQ ID NO: 1, but the substitution of A to G does not accompany a substitution of amino acid.

When homology with OWL by using Blast P [Nucleic Acids Res., 25, 3389, 1997] was examined, the human TCH204 variant 1 protein showed 74.2% homology to chicken calcium-dependent potassium channel cSlack (GenBank Accession #: AAM18770), 65.7% homology to rat calcium-dependent potassium channel Slack (Nature Neuroscience Vol. 1: 462, 1998; GenBank Accession #: AAC83350) and 63.2% homology to human potassium channel subunit protein KIAA1422 (GenBank Accession #: CAD 13242), respectively.

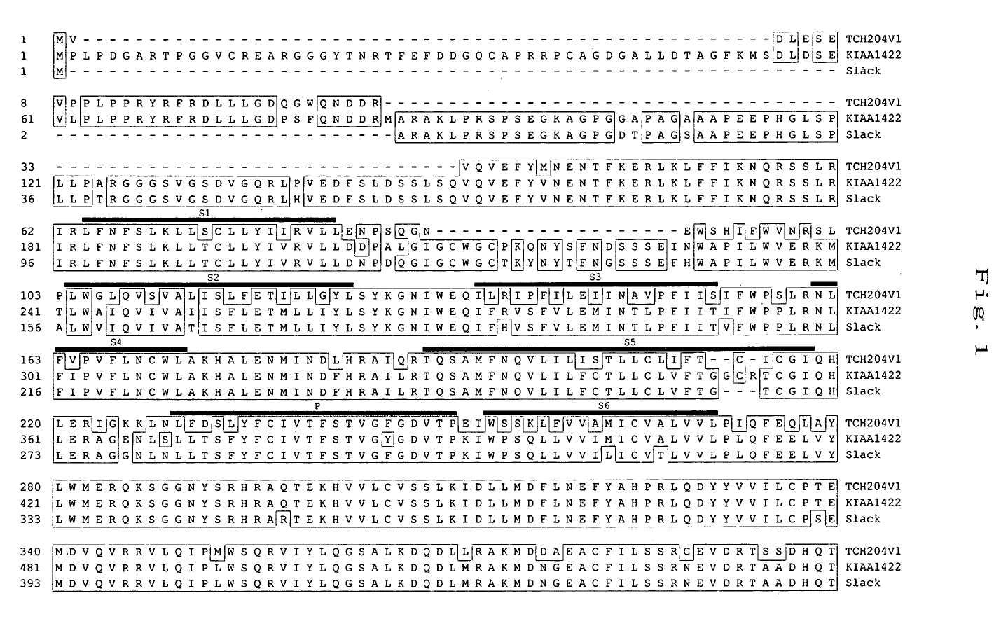

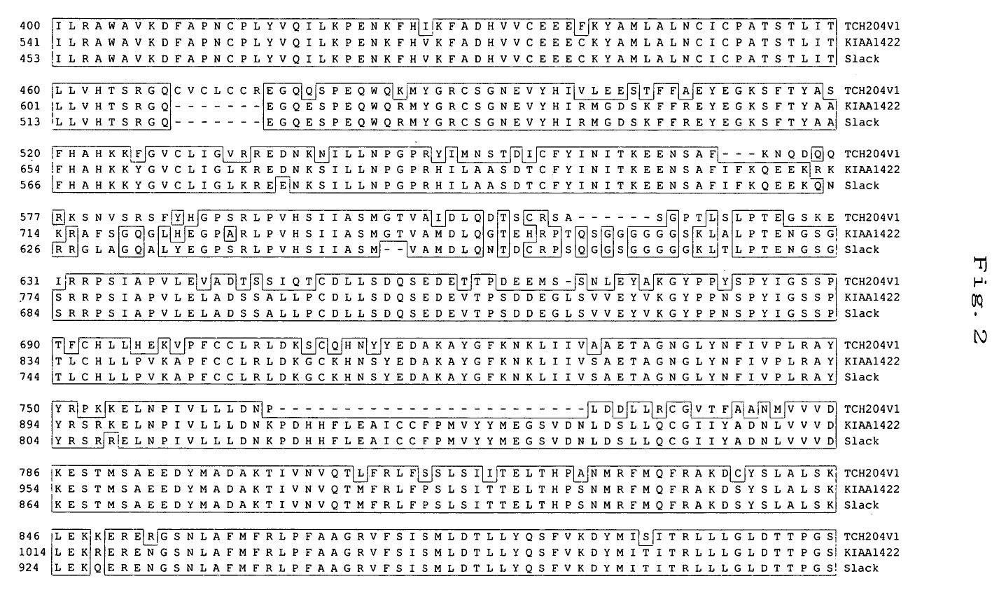

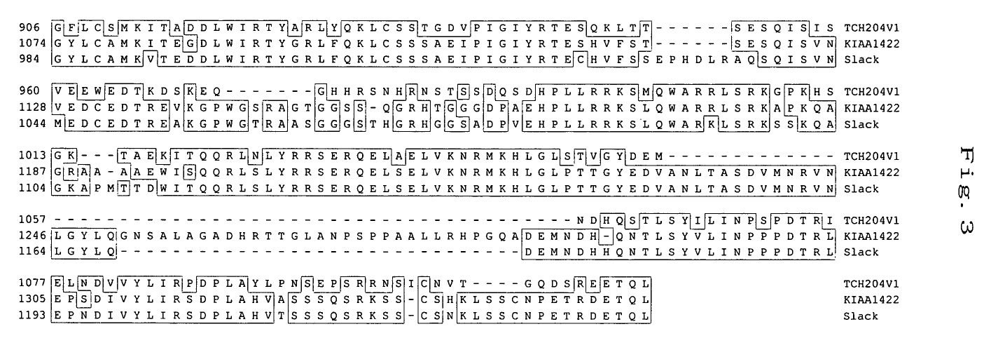

Further, from an analysis by hydropathy analysis software and a comparison of the amino acid sequence between Slack and KIAA1422 (FIGS. 1 through 3), it is presumable that the human TCH204 variant 1 protein is a six transmembrane type protein and possesses a structure having a pore region between the fifth transmembrane region and the sixth transmembrane region.

As described above, it is considered that cDNA encoding the human TCH204 variant 1 protein is novel gene belonging calcium dependent potassium channel. The calcium dependent potassium channel is classified into three groups, i.e., Big-K (conductance: 100-220 ps), Intermediate-K (conductance: 20-85 ps) and Small-K (conductance: 2-20 ps) by channel conductance. Table 1 shows a homology between the human TCH204 variant 1 protein and a representative molecule in each family. In Table 1, BK, IK and SK represent Big-K, Intermediate-K and Small-K, respectively.

TABLE 1

| Class | Molecule | Accession No. | Report | Homology |
|---|---|---|---|---|
| BK | hSlo | AAA85104 | Molecular Brain Res., | 14% |
| | | | Vol. 27, p. 189, 1994 | |
| IK | hIK1 | AAC23541 | Proc. Natl. Acad. Sci. | 11% |
| | | | USA, Vol. 94, p. 11651, | |
| | | | 1997 | |
| SK | hSK1 | AAB09562 | Science, Vol. 273, | 11% |
| | | | p. 1709, 1996 | |

### EXAMPLE 2

Cloning of cDNA Coding for Human TCH204 Variant 2 Gene

Using the following synthetic DNA primers and human testis-derived cDNA as a template, amplification by PCR method was performed. The reaction solution comprised of 2.0 μl of human testis Marathon-Ready cDNA (CLONTECH), 2 μM each of synthetic DNA primers (SEQ ID NO: 3 and SEQ ID NO: 4), 0.2 mM dNTPs, 0.125 μl of Pyrobest DNA Polymerase (Takara Shuzo) and Pyrobest buffer attached to the enzyme to make the total volume 20 μl. The cycle for amplification was carried out using a thermal cycler (PE Biosystems) by heating of 94° C. for 5 minutes, then a cycle set to include 94° C. for 20 seconds followed by 67° C. for 30 seconds and 72° C. for 5 minutes, which was repeated 35 times, and finally, incubation at 72° C. for 5 minutes. Then, the reaction solution comprised of 2 μl of the PCR reaction solution diluted 50-fold with DNase-, RNase-free distilled water, 2 μM each of synthetic DNA primers (SEQ ID NO: 5 and SEQ ID NO: 6), 0.2 mM dNTPs, 0.125 μl of Pyrobest DNA Polymerase (Takara Shuzo) and Pyrobest buffer attached to the enzyme to make the total volume 50 μl. The cycle for amplification was carried out using a thermal cycler (PE Biosystems) by heating of 94° C. for 5 minutes, then a cycle set to include 94° C. for 20 seconds followed by 67° C. for 30 seconds and 72° C. for 5 minutes, which was repeated 35 times, and finally, incubation at 72° C. for 5 minutes. The amplified DNA was isolated by 1.5% agarose gel electrophoresis and about 3.5 kb band was excised by razor blade. The DNA was recovered using the QIAquick Gel Extraction Kit (QIAGEN). The DNA was cloned into pCRII-TOPO vector in accordance with the protocol of TOPO TA Cloning Kit Dual Promoter (Invitrogen). After transformation of Escherichia coli TOP10 competent cell (Invitrogen) by introducing the above-mentioned vector, clones harboring cDNA insert fragment were selected on LB agar medium containing kanamycin and X-gal. All the white-colored clones were isolated with sterilized toothpick, and then the transformants were obtained. Respective clones were cultured in LB medium containing kanamycin for overnight. Subsequently, the plasmid DNA was prepared using QIAprep Miniprep (QIAGEN). The reaction for determination of the base sequence was carried out using BigDye Terminator Cycle Sequencing Ready Reaction Kit (Applied Biosystems). As a result, with the DNA sequencer, ABI PPISM 3100 Analyzer (Applied Biosystems), the base sequence of cDNA fragment inserted was decoded.

The 3496 bases (SEQ ID NO: 10) were inserted into the acquired plasmid, and ORF encoding 1111 amino acid residues (SEQ ID NO: 8) were present in the cDNA fragment (SEQ ID NO: 9). The protein containing the amino acid sequence represented by SEQ ID NO: 8 was designated human TCH204 variant 2 protein. The transformant having the plasmid containing the above-mentioned cDNA fragment (SEQ ID NO: 10) was designated Escherichia coli TOP10/pCRII-TCH204V2.

### EXAMPLE 3

Cloning of cDNA Coding for Human TCH204 Variant 3 Gene

Among a portion of the clones of Escherichia coli transformed with the same DNA as that of the PCR product used for obtaining the human TCH204 variant 2 gene in EXAMPLE 2, transformants harboring the human TCH204 variant 3 gene are present.

The 3346 bases (SEQ ID NO: 13) were inserted into the acquired plasmid, and ORF encoding 1061 amino acid residues (SEQ ID NO: 11) were present in the cDNA fragment (SEQ ID NO: 12). The protein containing the amino acid sequence represented by SEQ ID NO: 11 was designated human TCH204 variant 3 protein. The transformant having the plasmid containing the above-mentioned cDNA fragment (SEQ ID NO: 13) was designated Escherichia coli TOP 10/pCRII-TCH204V3.

### EXAMPLE 4

Cloning of cDNA Coding for Human TCH204 Variant 4 Gene

Using the following synthetic DNA primers and human testis-derived cDNA as a template, amplification by PCR method was performed. The reaction solution comprised of 2.0 μl of human testis Marathon-Ready cDNA (CLONTECH), 2 μM each of synthetic DNA primers (SEQ ID NO: 5 and SEQ ID NO: 6), 0.2 mM dNTPs, 0.125 μl of Pyrobest DNA Polymerase (Takara Shuzo) and Pyrobest buffer attached to the enzyme to make the total volume 20 μl. The cycle for amplification was carried out using a thermal cycler (PE Biosystems) by heating of 94° C. for 5 minutes, then a cycle set to include 94° C. for 10 seconds followed by 72° C. for 7 minutes, which was repeated 5 times, 94° C. for 10 seconds followed by 70° C. for 7 minutes, which was repeated 5 times, 94° C. for 10 seconds followed by 68° C. for 7 minutes, which was repeated 25 times, and finally, incubation at 68° C. for 10 minutes. Then, the reaction solution comprised of 2 μl of the PCR reaction solution diluted 50-fold with DNase-, RNase-free distilled water, 2 μM each of synthetic DNA primers (SEQ ID NO: 14 and SEQ ID NO: 6), 0.2 mM dNTPs, 0.125 μl of Pyrobest DNA Polymerase (Takara Shuzo) and Pyrobest buffer attached to the enzyme to make the total volume 50 μl. The cycle for amplification was carried out using a thermal cycler (PE Biosystems) by heating of 94° C. for 5 minutes, then a cycle set to include 94° C. for 10 seconds followed by 72° C. for 7 minutes, which was repeated 5 times, 94° C. for 10 seconds followed by 70° C. for 7 minutes, which was repeated 5 times, 94° C. for 10 seconds followed by 68° C. for 7 minutes, which was repeated 25 times, and finally, incubation at 72° C. for 5 minutes. The amplified DNA was isolated by 1.5% agarose gel electrophoresis and about 2.5 kb band was excised by razor blade. The DNA was recovered using the QIAquick Gel Extraction Kit (QIAGEN). The DNA was cloned into pCRII-TOPO vector in accordance with the protocol of TOPO TA Cloning Kit Dual Promoter (Invitrogen). After transformation of Escherichia coli TOP10 competent cell (Invitrogen) by introducing the above-mentioned vector, clones harboring cDNA insert fragment were selected on LB agar medium containing kanamycin and X-gal. All the white-colored clones were isolated with sterilized toothpick, and then the transformants were obtained. Respective clones were cultured in LB medium containing kanamycin for overnight. Subsequently, the plasmid DNA was prepared using QIAprep Miniprep (QIAGEN). The reaction for determination of the base sequence was carried out using BigDye Terminator Cycle Sequencing Ready Reaction Kit (Applied Biosystems). As a result, with the DNA sequencer, ABI PPISM 3100 Analyzer (Applied Biosystems), the base sequence of cDNA fragment inserted was decoded.

The 2576 bases (SEQ ID NO: 17) were inserted into the acquired plasmid, and ORF encoding 759 amino acid residues (SEQ ID NO: 15) were present in the cDNA fragment (SEQ ID NO: 16). The protein containing the amino acid sequence represented by SEQ ID NO: 15 was designated human TCH204 variant 4 protein. The transformant having the plasmid containing the above-mentioned cDNA fragment (SEQ ID NO: 17) was designated Escherichia coli TOP10/pCRII-TCH204V4.

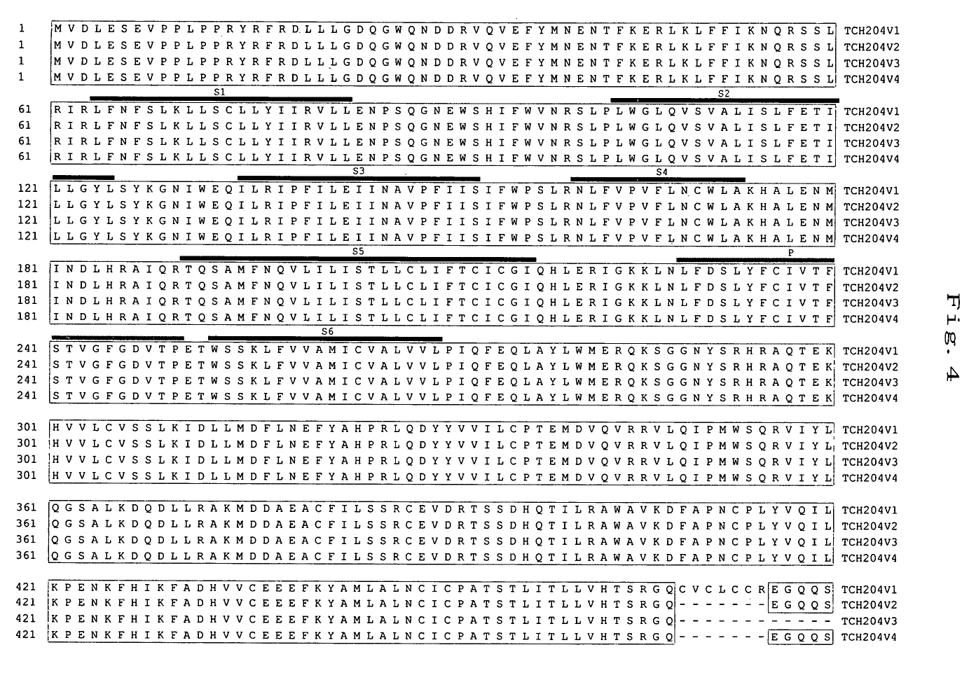

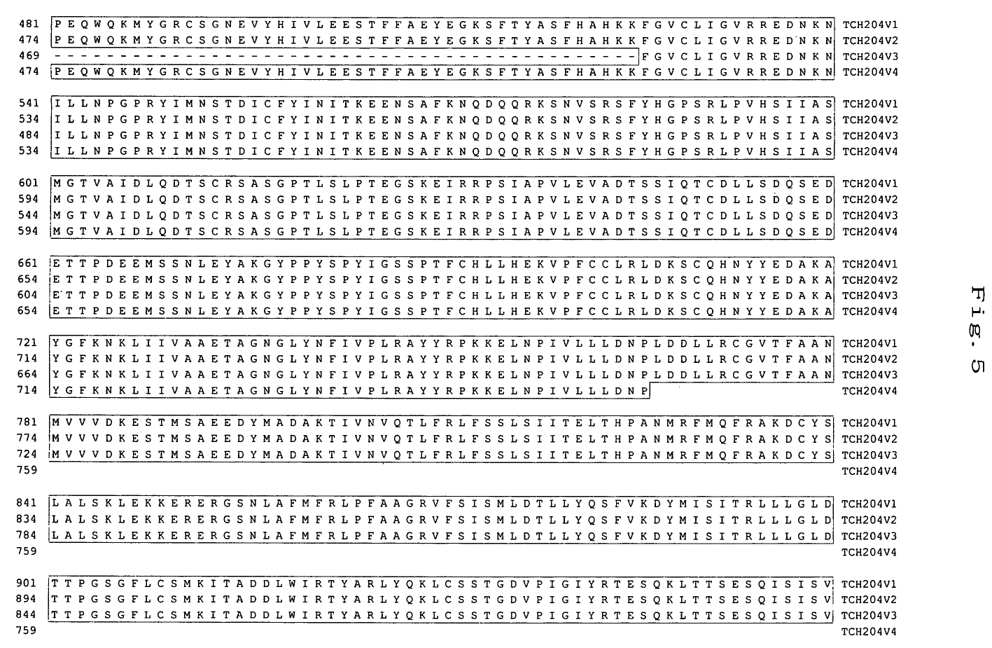

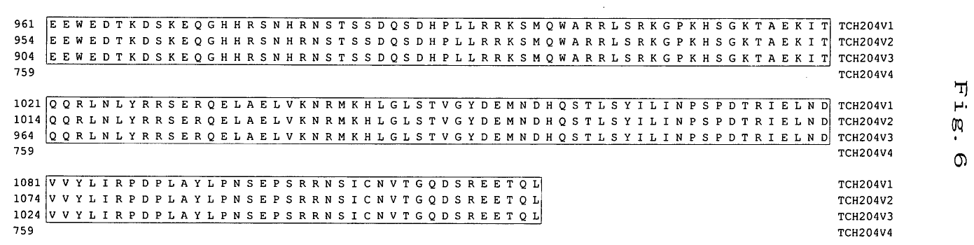

The respective amino acid sequences of the human TCH204 variant 1 protein, the human TCH204 variant 2 protein, the human TCH204 variant 3 and the human TCH204 variant 4 protein are shown in FIGS. 4 through 6.

### EXAMPLE 5

Cloning of cDNA Coding for Mouse TCH204 Variant 6 Gene

Using the following synthetic DNA primers and mouse brain-derived cDNA as a template, amplification by PCR method was performed. The reaction solution comprised of 2.0 μl of mouse brain Marathon-Ready cDNA (CLONTECH), 2 μM each of synthetic DNA primers (SEQ ID NO: 20 and SEQ ID NO: 21), 0.2 mM dNTPs, 0.125 μl of Pyrobest DNA Polymerase (Takara Shuzo) and Pyrobest buffer attached to the enzyme to make the total volume 20 μl. The cycle for amplification was carried out using a thermal cycler (PE Biosystems) by heating of 94° C. for 5 minutes, then a cycle set to include 94° C. for 20 seconds followed by 68° C. for 30 seconds and 72° C. for 5 minutes, which was repeated 40 times, and finally, incubation at 72° C. for 5 minutes. The amplified DNA was isolated by 1.5% agarose gel electrophoresis and about 3.5 kb band was excised by razor blade. The DNA was recovered using the QIAquick Gel Extraction Kit (QIAGEN). The DNA was cloned into pCRII-TOPO vector in accordance with the protocol of TOPO TA Cloning Kit Dual Promoter (Invitrogen). After transformation of Escherichia coli TOP10 competent cell (Invitrogen) by introducing the above-mentioned vector, clones harboring cDNA insert fragment were selected on LB agar medium containing kanamycin and X-gal. All the white-colored clones were isolated with sterilized toothpick, and then the transformants were obtained. Respective clones were cultured in LB medium containing kanamycin for overnight. Subsequently, the plasmid DNA was prepared using QIAprep Miniprep (QIAGEN). The reaction for determination of the base sequence was carried out using BigDye Terminator Cycle Sequencing Ready Reaction Kit (Applied Biosystems). As a result, with the DNA sequencer, ABI PPISM 3100 Analyzer (Applied Biosystems), the base sequence of cDNA fragment inserted was decoded. The 3498 bases were inserted into the acquired plasmid (SEQ ID NO: 22), and ORF encoding 1135 amino acid residues (SEQ ID NO: 18) were present in the cDNA fragment (SEQ ID NO: 19). The protein containing the amino acid sequence was designated human TCH204 variant 6 protein. The transformant having the plasmid containing the above-mentioned cDNA fragment was designated Escherichia coli TOP10/pCRII-mTCH204V6.

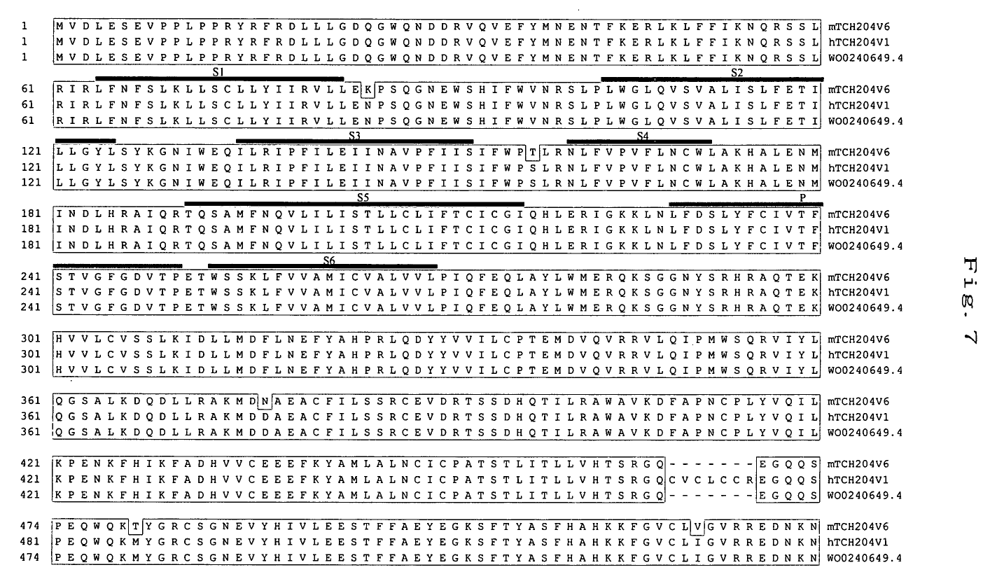

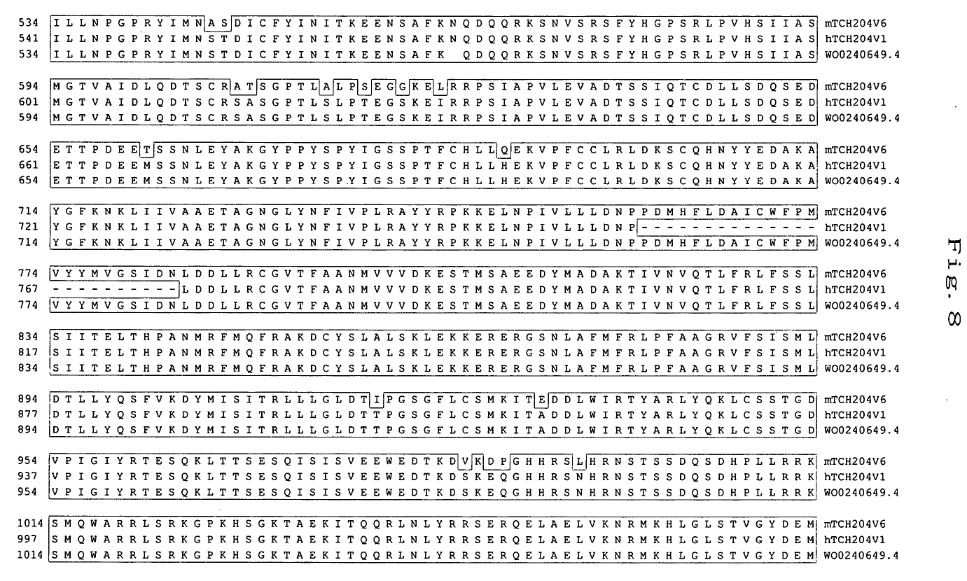

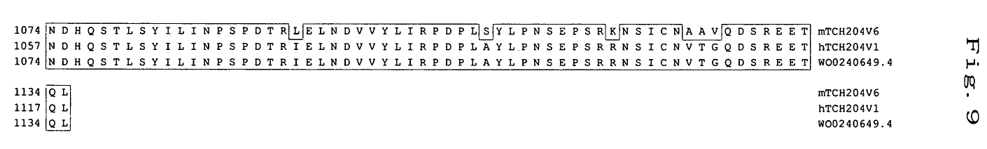

When homology with OWL by using Blast P [Nucleic Acids Res., 25, 3389, 1997] was examined, the human TCH204 variant 6 protein showed 96.4% homology to the human TCH204 variant 1 and 97.5% homology to the human calcium dependent potassium channel Slo4 (described in WO 02/40649 publication). Further, from an analysis by hydropathy analysis software and a comparison of the amino acid sequence between the human TCH204 and the Slo4, it is presumable that the protein is a six transmembrane type protein and possesses a structure having a membrane insertion region between the fifth transmembrane region and the sixth transmembrane region (FIGS. 7 through 9).

As described above, it is considered that cDNA encoding the mouse TCH204 variant 6 protein is novel gene belonging calcium dependent potassium channel.

The calcium dependent potassium channel is classified into three groups, i.e., Big-K (conductance: 100-220 ps), Intermediate-K (conductance: 20-85 ps) and Small-K (conductance: 2-20 ps) by channel conductance. Table 2 shows a homology between the mouse TCH204 variant 6 protein and a representative molecule from mouse in each family. In Table 2, BK, IK and SK represent Big-K, Intermediate-K and Small-K, respectively.

TABLE 2

| Class | Molecule | Accession No. | Report | Homology |
|---|---|---|---|---|
| BK | mSlo | NP_034740 | Science, Vol. 261, | 14% |
| | | | p. 221, 1993 | |
| IK | mIK1 | AAC32829 | | 13% |
| SK | mSK1 | Q9EQR3 | Biochim. Biophys. Acta | 12% |
| | | | Vol. 1518, p. 36, 2001 | |

### EXAMPLE 6

Cloning of cDNA Coding for Mouse TCH204 Variant 5 Gene

Among a portion of the clones of Escherichia coli transformed with the PCR product obtained in EXAMPLE 5, transformants harboring the mouse TCH204 variant 5 gene are present. The 3523 bases (SEQ ID NO: 25) were inserted into the acquired plasmid, and ORF encoding 1142 amino acid residues (SEQ ID NO: 23) were present in the cDNA fragment (SEQ ID NO: 24). The protein containing the amino acid sequence was designated mouse TCH204 variant 5 protein. The transformant having the plasmid containing the above-mentioned cDNA fragment was designated Escherichia coli TOP10/pCRII-mTCH204V5.

### EXAMPLE 7

Cloning of cDNA Coding for Mouse TCH204 Variant 2 Gene

Among a portion of the clones of Escherichia coli transformed with the PCR product obtained in EXAMPLE 5, transformants harboring the mouse TCH204 variant 2 gene are present. The 3430 bases (SEQ ID NO: 28) were inserted into the acquired plasmid, and ORF encoding 1111 amino acid residues (SEQ ID NO: 26) were present in the cDNA fragment (SEQ ID NO: 27). The protein containing the amino acid sequence was designated mouse TCH204 variant 2 protein. The transformant having the plasmid containing the above-mentioned cDNA fragment was designated Escherichia coli TOP10/pCRII-mTCH204V2.

Whereas the 2548th base in the sequence represented by SEQ ID NO: 27 was C, A at the 2745th position and A at the 2943rd position, the corresponding base of the other human TCH204 variant obtained in EXAMPLES 5 and 6 described above was T, G and G, respectively. It is considered that the base substitutions were occurred by SNP. In addition, the respective base substitutions do not accompany a substitution of amino acid.

### EXAMPLE 8

Analysis for Tissue Distribution of Gene Product of each Human TCH204 Variant

For separately quantifying an expression level of each variant gene, sequences for primers and probes were designed from the sequences of respective human TCH204 variants.

For the human TCH204 variant 1, the synthetic DNA primers of SEQ ID NO: 29 and SEQ ID NO: 30, and the synthetic DNA probe for TaqMan PCR of SEQ ID NO: 31; for the human TCH204 variant 2, the synthetic DNA primers of SEQ ID NO: 32 and SEQ ID NO: 30, and the synthetic DNA probe for TaqMan PCR of SEQ ID NO: 33; for the human TCH204 variant 3, the synthetic DNA primers of SEQ ID NO: 32 and SEQ ID NO: 34, and the synthetic DNA probe for TaqMan PCR of SEQ ID NO: 35; for the human TCH204 variant 4, the synthetic DNA primers of SEQ ID NO: 36 and SEQ ID NO: 37, and the synthetic DNA probe for TaqMan PCR of SEQ ID NO: 38; for the human TCH204 variant 5, the synthetic DNA primers of SEQ ID NO: 39 and SEQ ID NO: 40, and the synthetic DNA probe for TaqMan PCR of SEQ ID NO: 41; and for the human TCH204 variant 6, the synthetic DNA primers of SEQ ID NO: 42 and SEQ ID NO. 43, and the synthetic DNA probe for TaqMan PCR of SEQ ID NO: 44; these synthetic DNA were designed, respectively.

In addition, the human TCH204 variant 5 shows the sequence disclosed as Potassium Channel 54414 in WO 02/055701 publication, and the human TCH204 variant 6 shows the sequence disclosed as human Slo4 in WO 02/40469 publication. These sequences have at least 95% homology to the human TCH204 variant 1, variant 2, variant 3 and variant 4, respectively, but did not coincide with the sequence thereof.

When we examined if the interested variant can only be quantified using the primers and probe, which was designed for quantification of the specific variant, with a plasmid DNA containing the base sequence encoding whole length amino acid sequence of each human TCH204 variant as a standard DNA, in the case of primers and probes for the human TCH204 variant 1, variant 3, variant 4, variant 5 and variant 6, only the interested variant were determined. On the other hand, it was found that in the case of primers and probe for the human TCH204 variant 2, the human TCH204 variant 1, variant 3, variant 4, variant 5 and variant 6 were determined in addition to the human TCH204 variant 2. However, due to the relation for the genetic sequences between the human TCH204 variant 2 and the other human TCH204 variants, merger of the human TCH204 variant 1, variant 3, variant 4, variant 5 and variant 6 was inevitable. Therefore, the quantification for the human TCH204 variant 2 was carried out with the inclusion of the human TCH204 variant 1, variant 3, variant 4, variant 5 and variant 6. The expression level of the human TCH204 variant 2 was estimated by subtracting respective expression level of the other human TCH204 variants from the aforementioned result.

The reaction composition for the quantification by the TaqMan PCR system was as follows: 0.8 μl of human various tissue-derived cDNA (CLONTECH, Human MTC Panel I and II), 10 μl of TaqMan Universal PCR Master Mix (Applied Biosystems), 0.2 μM each of synthetic DNA primers designed for the quantification of each human TCH204 variant and 0.2 μM synthetic DNA probe for the TaqMan to make the total volume 20 μl. The reaction composition for the quantification of glyceraldehyde phosphate dehydrogenase (GAPDH) in each cDNA was as follows: 0.8 μl of human various tissue-derived cDNA (CLONTECH, Human MTC Panel I and II), 10 μl of TaqMan Universal PCR Master Mix (Applied Biosystems), 0.2 μM each of synthetic DNA primers attached to the GAPDH Control Reagent 402869 (Applied Biosystems) and 0.1 μM synthetic DNA probe for the TaqMan attached to the same kit to make the total volume 20 μl. For the reaction, ABI PRISM 7900 Sequence Detection System was used. The reaction was performed at 50° C. for 2 minute and 95° C. for 10 minutes and then repeated 40 cycles set to include 95° C. for 15 seconds and 60° C. for one minute. Simultaneously at completion of the reaction, the quantification was conducted. The various human tissue-derived cDNAs used for determination were listed in Table 3.

TABLE 3

| Sample Name | Human Tissues, which are included in sample |
|---|---|
| Human MTC Panel I | Heart, Brain, Placenta, Lung, Liver, |
| | Skeletal muscle, Kidney, Pancreas |
| Human MTC Panel II | Spleen, Thymus, Prostate, Testis, Ovary, |
| | Small intestine, Colon, Peripheral |
| | Blood Leukocyte |

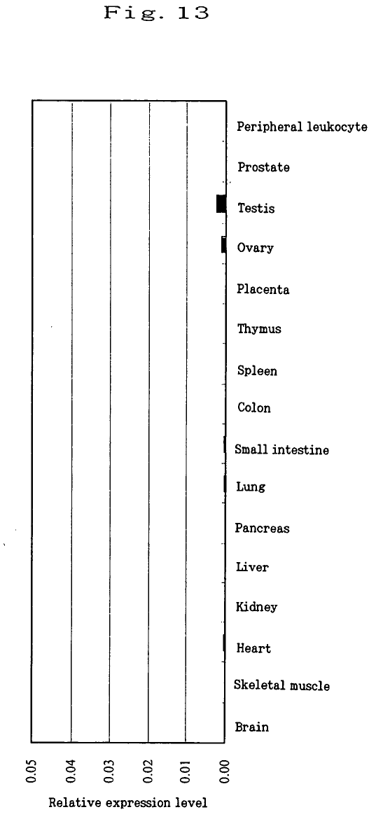

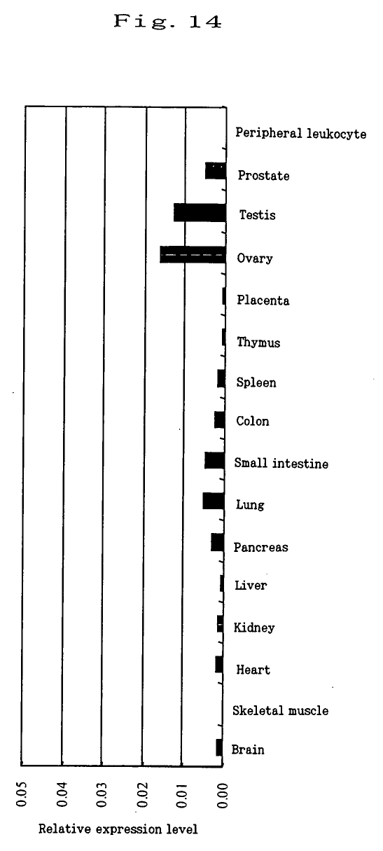

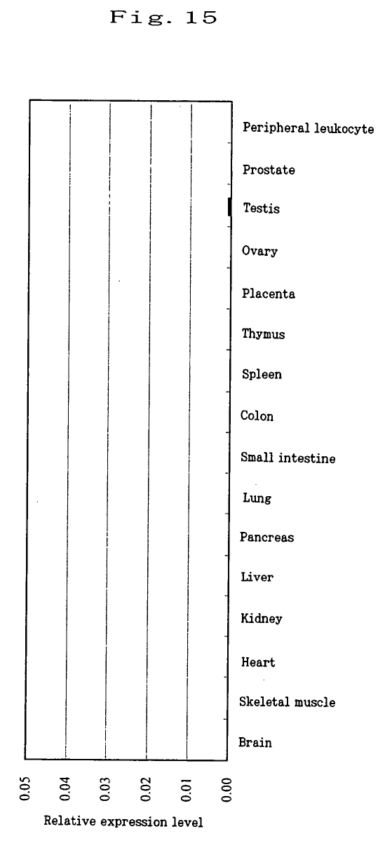

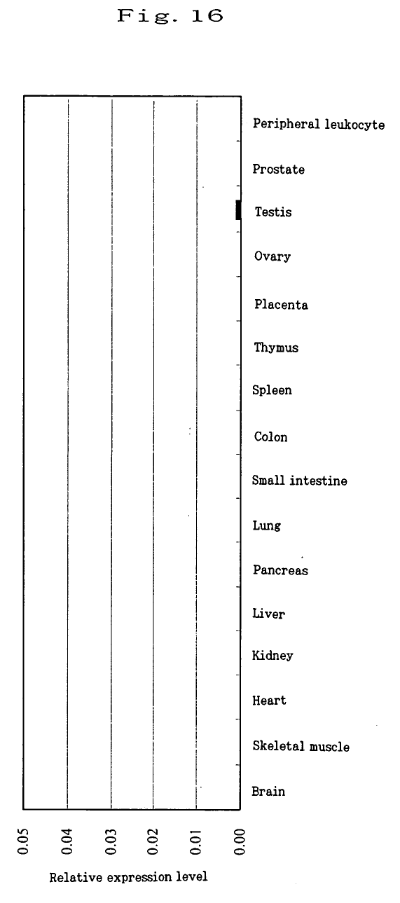

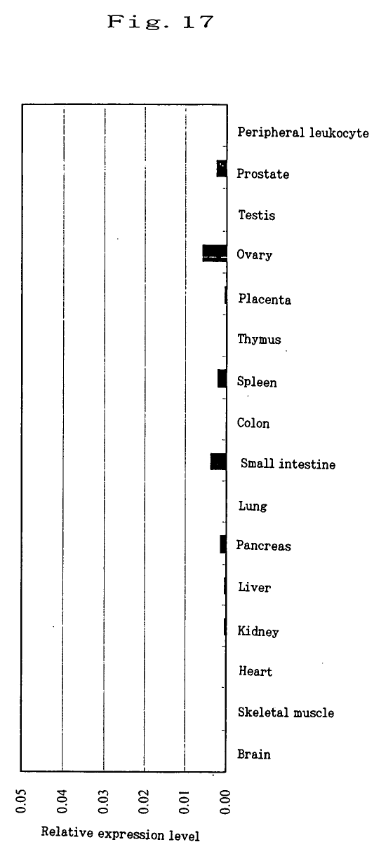

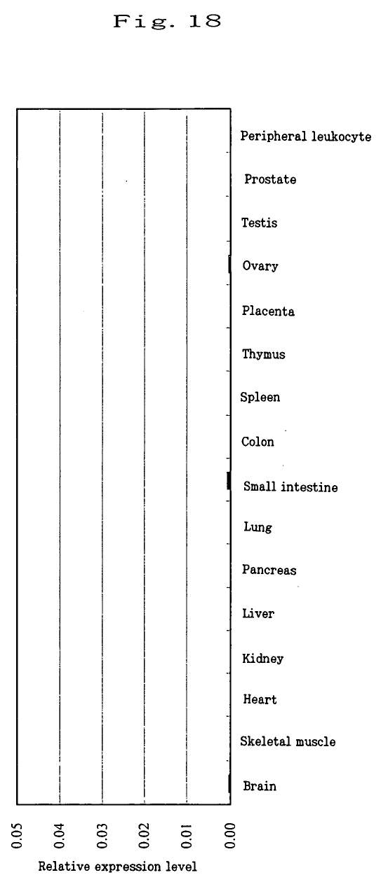

The results are shown in FIGS. 13 through 18.

The results are represented by the relative expression level of each human TCH204 variant against the expression level of the GAPDH gene in each cDNA sample, i.e., the values obtained by dividing the expression level of each human TCH204 variant per unit volume of cDNA (copies/μl) into the expression level of the GAPDH gene per unit volume of cDNA (copies/μl).

The human TCH204 variant 1 was slightly expressed in ovary and testis.

In the case of the human TCH204 variant 2 system, the expression was detected in lung, small intestine, ovary, testis and prostate. As described above, since the human TCH204 variant 2 is quantified in the inclusion of the expression level of the other human TCH204 variants, it should be estimated by subtracting the expression level of the other human TCH204 variants. By this treatment, it was presumed that majority of the expression detected in lung, ovary and testis is derived from the human TCH204 variant 2.

In the human TCH204 variant 3, no expression was detected in the tissues other than testis. Further, the expression in testis was slight.

In the human TCH204 variant 4, as well as the case of the human TCH204 variant 3, no expression was detected in the tissues other than testis. Further, the expression in testis was slight.

The expression of the human TCH204 variant 5 was detected in small intestine, spleen, ovary and prostate.

The expression of the human TCH204 variant 6 was slightly detected in small intestine and ovary.

### EXAMPLE 9

Electrophysiological Properties of Human TCH204 Variant 2 Gene Product

To determine if an ion channel is formed by the human TCH204 variant 2, whole cell recording by fixing membrane potential was performed using patch-clamp techniques.

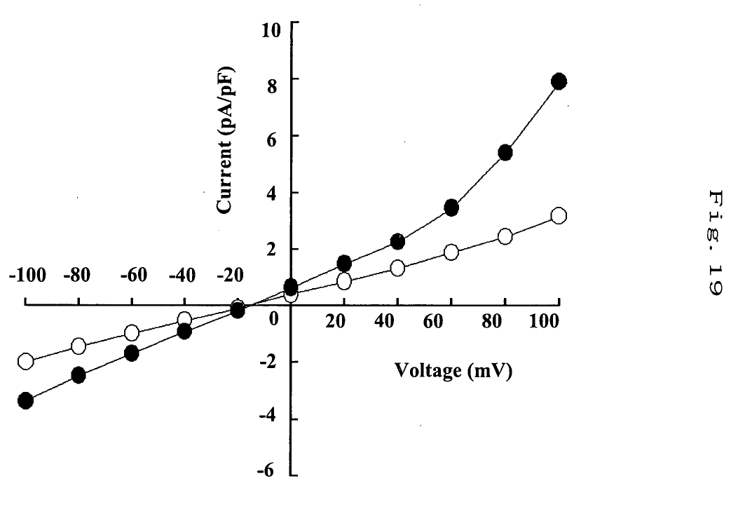

The human TCH204 variant 2, which was inserted into vector pcDNA3.1(+) (Invitrogen) using Nucleofector (AMAXA), was transiently expressed in CHO-K1 cells (7 examples). After 24 or 48 hours, measurement was done. As a control, the cells, in which only the vector was introduced, were used (5 examples). A glass tube (GC150-10, Harvard-Clerk) was used as an electrode for patch. Further, the electrode was processed to make the chip resistance 5-10 megaohm. The compositions of filling solution and outer solution of the cells was in accordance with the description in the reference (Nature Neurosci., Vol. 1, pp. 462-469, 1998). The filling solution consisted of 32.5 mM KCl, 97.5 mM K-gluconate, 0.1 mM CaCL₂ and 10 mM HEPES (pH was adjusted to 7.2 with KOH). The outer solution of the cells consisted of 140 mM NaCl, 3.0 mM KCl, 1.0 mM CaCl₂-2H₂O, 29 mM D-glucose, 25 mM HEPES and 1 μM tetrodotoxin (pH was adjusted to 7.4 with NaOH). For whole cell recording, the membrane potential was held at −80 mV with amplifier for patch, Axopatch 200B (Axon Instruments), and sampling at 5 kHz was carried out through A/D converter, which is filtered at 2 kHz (Digidata 1320A, Axon Instruments). These recording and data processing are totally performed on pCLAMP8 software (Axon Instruments). When a step pulse every 20 mV from −100 mV to +100 mV for 300 mili-seconds was given to the human TCH204 variant 2-introduced cells at 2 seconds-intervals, generation of potential-dependent electric current, which possesses an extrovert rectified component as compared to that of the control cells, was detected. The results are shown in FIG. 19. Isostatic potential for this current demonstrates a dependency on a K⁺ ion concentration in the outer solution of the cells. From the results, it was demonstrated that the human TCH204 variant 2 forms ion channel, presumably K⁺ ion-selective potential-dependent ion channel.

### INDUSTRIAL APPLICABILITY

The protein, the polynucleotide, the antibody and the like of the present invention are useful as a diagnostic marker for alimentary diseases (e.g., irritable bowel syndrome, chronic ulcerative colitis, Crohn's disease, ischemic colitis, gastritis, peptic ulcer, proctitis, diarrhea, reflux esophagitis, duodenitis, etc.), reproductive system diseases (e.g., prostate hyperplasia, prostatitis, testis neurosis, ovarian cystoma, etc.), respiratory diseases (e.g., chronic obstructive pulmonary disease, asthma, etc.), central nervous system diseases (e.g., Alzheimer's disease, parkinsonian syndrome, schizophrenia, cerebrovascular dementia, cerebral ischemia, convulsions, etc.), diabetes mellitus, hypertension, circulatory diseases (e.g., heart failure, arrhythmia, long QT syndrome, arteriosclerosis, angina, etc.), pancreatic diseases (e.g., pancreatitis, hypopancreatism such as cystic fibrosis pancreas, etc.), liver diseases (e.g., cirrhosis hepatic, etc.), kidney diseases (e.g., kidney failure, uremia, etc.), cancer (e.g., lung cancer, kidney cancer, liver cancer, non-small cell lung cancer, ovarian cancer, prostate cancer, stomach cancer, bladder cancer, breast cancer, cancer of uterine cervix, colon cancer, rectum cancer, pancreatic cancer, tymoma, testicular tumor, esophageal cancer, myosarcoma, etc.), or the like. Compounds that activate or inhibit the activities of the protein, the polynucleotide or the antibody, compounds that activate or inhibit the expression of the gene encoding the protein, compounds that activate or inhibit the expression of the protein and the like, which are obtained by the screening method using the protein, can be used as a prophylactic and/or therapeutic agent for alimentary diseases (e.g., irritable bowel syndrome, chronic ulcerative colitis, Crohn's disease, ischemic colitis, gastritis, peptic ulcer, proctitis, diarrhea, reflux esophagitis, duodenitis, etc.), reproductive system diseases (e.g., prostate hyperplasia, prostatitis, testis neurosis, ovarian cystoma, etc.), respiratory diseases (e.g., chronic obstructive pulmonary disease, asthma, etc.), central nervous system diseases (e.g., Alzheimer's disease, parkinsonian syndrome, schizophrenia, cerebrovascular dementia, cerebral ischemia, convulsions, etc.), diabetes mellitus, hypertension, circulatory diseases (e.g., heart failure, arrhythmia, long QT syndrome, arteriosclerosis, angina, etc.), pancreatic diseases (e.g., pancreatitis, hypopancreatism such as cystic fibrosis pancreas, etc.), liver diseases (e.g., cirrhosis hepatic, etc.), kidney diseases (e.g., kidney failure, uremia, etc.), cancer (e.g., lung cancer, kidney cancer, liver cancer, non-small cell lung cancer, ovarian cancer, prostate cancer, stomach cancer, bladder cancer, breast cancer, cancer of uterine cervix, colon cancer, rectum cancer, pancreatic cancer, tymoma, testicular tumor, esophageal cancer, myosarcoma, etc.), or the like. Preferably, they can be used as a prophylactic and/or therapeutic agent for alimentary diseases, reproductive system diseases, respiratory diseases, or the like.

1. An isolated protein comprising the same or substantially the same amino acid sequence as the amino acid sequence represented by SEQ ID NO: 1, SEQ ID NO: 8, SEQ ID NO: 11 or SEQ ID NO: 15, or a salt thereof.

2. The protein according to claim 1 or a salt thereof, wherein the protein comprising the same or substantially the same amino acid sequence as the amino acid sequence represented by SEQ ID NO: 1, SEQ ID NO: 8, SEQ ID NO: 11 or SEQ ID NO: 15 is a protein consisting of the amino acid sequence represented by SEQ ID NO: 18, SEQ ID NO: 23 or SEQ ID NO: 26.

3. An isolated protein consisting of the amino acid sequence represented by SEQ ID NO: 1, or a salt thereof.

4. An isolated protein consisting of the amino acid sequence represented by SEQ ID NO: 8, or a salt thereof.

5. An isolated protein consisting of the amino acid sequence represented by SEQ ID NO: 11, or a salt thereof.

6. An isolated protein consisting of the amino acid sequence represented by SEQ ID NO: 15, or a salt thereof.

7. A partial peptide of the protein according to claim 1, or a salt thereof.

8. An isolated polynucleotide comprising the polynucleotide encoding the protein according to claim 1 or a partial peptide thereof.

9. The polynucleotide according to claim 8, which is a DNA.

10. An isolated polynucleotide consisting of the nucleic acid base sequence represented by SEQ ID NO: 2, SEQ ID NO: 9, SEQ ID NO: 12 or SEQ ID NO: 16.

11. An isolated polynucleotide consisting of the nucleic acid base sequence represented by SEQ ID NO: 19, SEQ ID NO: 24 or SEQ ID NO: 27.

12. A recombinant vector comprising the polynucleotide according to claim 8.

13. A transformant, which is transformed with the recombinant vector according to claim 12.

14. A method for manufacturing the protein according to claim 1 or the partial peptide or salts thereof, which comprises culturing a suitable host cell transformed with an expressible recombinant DNA vector which encodes for said protein under suitable conditions and for sufficient time to express said protein, producing and accumulating the protein and collecting said protein.

15. A pharmaceutical composition comprising the protein according to claim 1 or the partial peptide or salts thereof and a pharmaceutically acceptable carrier, excipient or diluent.

16. A pharmaceutical composition comprising the polynucleotide according to claim 8 and a pharmaceutically acceptable carrier, excipient or diluent.

17. A diagnostic reagent comprising the polynucleotide according to claim 8.

18. An antibody to the protein according to claim 1 or the partial peptide or salts thereof.

19. A diagnostic reagent comprising the antibody according to claim 18.

20. A pharmaceutical composition comprising the antibody according to claim 18 and a pharmaceutically acceptable carrier, excipient or diluent.

21. An isolated polynucleotide comprising a nucleic acid base sequence complementary or substantially complementary to the nucleic acid base sequence of the polynucleotide according to claim 8 or a portion thereof.

22. A pharmaceutical composition comprising the polynucleotide according to claim 21 and a pharmaceutically acceptable carrier, excipient or diluent.

23. A method for screening a compound, or a salt thereof, that enhances or inhibits activities of the protein according to claim 1 or the partial peptide or salts thereof, which comprises using the protein according to claim 1 or the partial peptide or salts thereof in a screening assay.

24. A kit for screening a compound, or a salt thereof, that enhances or inhibits activities of the protein according to claim 1 or the partial peptide or salts thereof, which comprises the protein according to claim 1 or the partial peptide or salts thereof.

25. A compound, or a salt thereof, that enhances or inhibits activities of the protein comprising the same or substantially the same amino acid sequence as the amino acid sequence represented by SEQ ID NO: 1, SEQ ID NO: 8, SEQ ID NO: 11 or SEQ ID NO: 15, or the partial peptide or salts thereof, which is obtainable by using the screening method according to claim 23.

26. A pharmaceutical composition comprising the compound according to claim 25 or a salt thereof and a pharmaceutically acceptable carrier, excipient or diluent.

27. A method for screening a compound that enhances or inhibits an expression of the gene encoding the protein comprising the same or substantially the same amino acid sequence as the amino acid sequence represented by SEQ ID NO: 1, SEQ ID NO: 8, SEQ ID NO: 11 or SEQ ID NO: 15, which comprises using the polynucleotide according to claim 8 in a screening assay.

28. A kit for screening a compound that enhances or inhibits an expression of the gene encoding the protein comprising the same or substantially the same amino acid sequence as the amino acid sequence represented by SEQ ID NO: 1, SEQ ID NO: 8, SEQ ID NO: 11 or SEQ ID NO: 15, which comprises the polynucleotide according to claim 8.

29. A compound that enhances or inhibits an expression of the gene encoding the protein comprising the same or substantially the same amino acid sequence as the amino acid sequence represented by SEQ ID NO: 1, SEQ ID NO: 8, SEQ ID NO: 11 or SEQ ID NO: 15, which is obtainable by the screening method according to claim 27.

30. A pharmaceutical composition comprising the compound according to claim 29 or a salt thereof and a pharmaceutically acceptable carrier, excipient or diluent.

31. A method for quantifying the protein comprising the same or substantially the same amino acid sequence as the amino acid sequence represented by SEQ ID NO: 1, SEQ ID NO: 8, SEQ ID NO: 11 or SEQ ID NO: 15, which comprises using the antibody according to claim 18.

32. A method for diagnosing a disease associated with the function of the protein protein comprising the same or substantially the same amino acid sequence as the amino acid sequence represented by SEQ ID NO: 1, SEQ ID NO: 8, SEQ ID NO: 11 or SEQ ID NO: 15, which comprises using the quantification method according to claim 31.

33. A method for screening a compound that enhances or inhibits an expression of the gene encoding the protein protein comprising the same or substantially the same amino acid sequence as the amino acid sequence represented by SEQ ID NO: 1. SEQ ID NO: 8. SEQ ID NO: 11 or SEQ ID NO: 15, which comprises using the antibody according to claim 18 in a screening assay.

34. A kit for screening a compound that enhances or inhibits an expression of the gene encoding the protein comprising the same or substantially the same amino acid sequence as the amino acid sequence represented by SEQ ID NO: 1, SEQ ID NO: 8, SEQ ID NO: 11 or SEQ ID NO: 15, which comprises the polynucleotide according to claim 18.

35. A compound that enhances or inhibits an expression of the gene encoding the protein comprising the same or substantially the same amino acid sequence as the amino acid sequence represented by SEQ ID NO: 1, SEQ ID NO: 8, SEQ ID NO: 11 or SEQ ID NO: 15, which is obtainable by the screening method according to claim 33.

36. A pharmaceutical composition comprising the compound according to claim 35 or a salt thereof and a pharmaceutically acceptable carrier, excipient or diluent.

37. (canceled)

38. (canceled)

39. (canceled)

40. (canceled)

41. A pharmaceutical composition for treating alimentary diseases, genital diseases or respiratory diseases in a mammal in need thereof, comprising:
a) an isolated polynucleotide which encodes for a protein comprising the same or substantially the same amino acid sequence as the amino acid sequence represented by SEQ ID NO: 1, SEQ ID NO: 8, SEQ ID NO: 11 or SEQ ID NO: 15;
b) an isolated antibody which specifically binds to a protein comprising the same or substantially the same amino acid sequence as the amino acid sequence represented by SEQ ID NO: 1, SEQ ID NO: 8, SEQ ID NO: 11 or SEQ ID NO: 15;
c) an isolated polynucleotide which is complementary to a polynucleotide which encodes for a protein comprising the same or substantially the same amino acid sequence as the amino acid sequence represented by SEQ ID NO: 1, SEQ ID NO: 8, SEQ ID NO: 11 or SEQ ID NO: 15; or
d) a compound, or a salt thereof, that enhances or inhibits activities of a protein comprising the same or substantially the same amino acid sequence as the amino acid sequence represented by SEQ ID NO: 1, SEQ ID NO: 8, SEQ ID NO: 11 or SEQ ID NO: 15;
a. where said compound is obtainable by a screening method using a protein comprising the same or substantially the same amino acid sequence as the amino acid sequence represented by SEQ ID NO: 1, SEQ ID NO: 8, SEQ ID NO: 11 or SEQ ID NO: 15 in said assay;
b. where said compound is obtainable by a screening method using a polynucleotide which encodes for a protein comprising the same or substantially the same amino acid sequence as the amino acid sequence represented by SEQ ID NO: 1, SEQ ID NO: 8, SEQ ID NO: 11 or SEQ ID NO: 15 in said assay; or
c. where said compound is obtainable by a screening method using an antibody which specifically binds a protein comprising the same or substantially the same amino acid sequence as the amino acid sequence represented by SEQ ID NO: 1, SEQ ID NO: 8, SEQ ID NO: 11 or SEQ ID NO: 15 in said assay;
and a pharmaceutically acceptable carrier, excipient or diluent.

42. A method for diagnosing alimentary diseases, genital diseases or respiratory diseases in a mammal in need thereof comprising contacting a diagnostic reagent comprising:
a) an isolated polynucleotide which encodes for a protein comprising the same or substantially the same amino acid sequence as the amino acid sequence represented by SEQ ID NO: 1, SEQ ID NO: 8, SEQ ID NO: 11 or SEQ ID NO: 15;
b) an isolated antibody which specifically binds to a protein comprising the same or substantially the same amino acid sequence as the amino acid sequence represented by SEQ ID NO: 1, SEQ ID NO: 8, SEQ ID NO: 11 or SEQ ID NO: 15;
c) an isolated polynucleotide which is complementary to a polynucleotide which encodes for a protein comprising the same or substantially the same amino acid sequence as the amino acid sequence represented by SEQ ID NO: 1, SEQ ID NO: 8, SEQ ID NO: 11 or SEQ ID NO: 15;
to a cell, tissue, sample or specimen from said mammal.

43. A method for treating alimentary diseases, genital diseases or respiratory diseases comprising administering to a mammal in need thereof, and effective amount of a pharmaceutical composition comprising a compound, or a salt thereof, that enhances or inhibits activities of a protein comprising the same or substantially the same amino acid sequence as the amino acid sequence represented by SEQ ID NO: 1, SEQ ID NO: 8, SEQ ID NO: 11 or SEQ ID NO: 15;
a) where said compound is obtainable by a screening method using a protein comprising the same or substantially the same amino acid sequence as the amino acid sequence represented by SEQ ID NO: 1, SEQ ID NO: 8, SEQ ID NO: 11 or SEQ ID NO: 15 in said assay;
b) where said compound is obtainable by a screening method using a polynucleotide which encodes for a protein comprising the same or substantially the same amino acid sequence as the amino acid sequence represented by SEQ ID NO: 1, SEQ ID NO: 8, SEQ ID NO: 11 or SEQ ID NO: 15 in said assay; or
c) where said compound is obtainable by a screening method using an antibody which specifically binds a protein comprising the same or substantially the same amino acid sequence as the amino acid sequence represented by SEQ ID NO: 1, SEQ ID NO: 8, SEQ ID NO: 11 or SEQ ID NO: 15 in said assay.

44. A method for making a pharmaceutical composition for treating alimentary diseases, genital diseases or respiratory diseases in a mammal in need thereof, said method comprising combining an effective amount of a compound, or a salt thereof, that enhances or inhibits activities of a protein comprising the same or substantially the same amino acid sequence as the amino acid sequence represented by SEQ ID NO: 1, SEQ ID NO: 8, SEQ ID NO: 11 or SEQ ID NO: 15;
a) where said compound is obtainable by a screening method using a protein comprising the same or substantially the same amino acid sequence as the amino acid sequence represented by SEQ ID NO: 1, SEQ ID NO: 8, SEQ ID NO: 11 or SEQ ID NO: 15 in said assay;
b) where said compound is obtainable by a screening method using a polynucleotide which encodes for a protein comprising the same or substantially the same amino acid sequence as the amino acid sequence represented by SEQ ID NO: 1, SEQ ID NO: 8, SEQ ID NO: 11 or SEQ ID NO: 15 in said assay; or
c) where said compound is obtainable by a screening method using an antibody which specifically binds a protein comprising the same or substantially the same amino acid sequence as the amino acid sequence represented by SEQ ID NO: 1, SEQ ID NO: 8, SEQ ID NO: 11 or SEQ ID NO: 15 in said assay;
with a pharmaceutically acceptable carrier, excipient or diluent.
